(19) United States
(12) Patent Application Publication
(10) Pub. No.: US 2023/0304061 A1
(43) Pub. Date: Sep. 28, 2023

(54) MICROORGANISM THAT PRODUCES USEFUL SUBSTANCE AND METHOD FOR PRODUCING USEFUL SUBSTANCE

(71) Applicant: KANEKA CORPORATION, Osaka-shi, Osaka (JP)

(72) Inventors: Shingo KOBAYASHI, Takasago-shi, Hyogo (JP); Nobuhiko DOJUN, Takasago-shi, Hyogo (JP); Misato MATSUI, Takasago-shi, Hyogo (JP); Naoaki TAOKA, Takasago-shi, Hyogo (JP)

(73) Assignee: KANEKA CORPORATION, Osaka-shi, Osaka (JP)

(21) Appl. No.: 17/918,857

(22) PCT Filed: Apr. 23, 2021
(86) PCT No.: PCT/JP2021/016499

§ 371 Date: Oct. 13, 2022

(30) Foreign Application Priority Data
Apr. 28, 2020 (JP) 2020-079058

(51) Int. Cl. C12P 21/02; C12N 9/10; C12N 9/48; C12N 9/02; C12N 9/88

(52) U.S. Cl. CPC C12P 21/02; C12N 9/0051; C12N 9/1029; C12N 9/485; C12N 9/88; C12Y 108/01007; C12Y 203/0103; C12Y 304/11004; C12Y 304/19013; C12Y 401/99001

(57) ABSTRACT

This invention is intended to improve glutathione productivity by fermentation using microorganisms. This invention relates to a microbial strain capable of overexpression of γ-glutamylcysteine, bis-γ-glutamylcystine, γ-glutamylcystine, reduced glutathione, and/or oxidized glutathione in which the expression level of a gene encoding serine-O-acetyltransferase (EC:2.3.1.30) is enhanced and a method involving the use of such microbial strain.

### TECHNICAL FIELD

One or more embodiments of the present invention relate to a microbial strain capable of overproduction of γ-glutamylcysteine, bis-γ-glutamylcystine, γ-glutamylcystine, reduced glutathione, and/or oxidized glutathione.

Other one or more embodiments of the present invention relate to a method for producing γ-glutamylcysteine, bis-γ-glutamylcystine, γ-glutamylcystine, reduced glutathione, and/or oxidized glutathione.

### BACKGROUND ART

Glutathione is a peptide comprising 3 amino acids; i.e., L-cysteine, L-glutamic acid, and glycine, which is a compound that exists in a wide variety of organisms, such as animals including humans, plants, and microorganisms and is important for organisms that play a role in, for example, active oxygen elimination, detoxication, and amino acid metabolism.

Glutathione is present in an organism in either of a glutathione reduced form in which the thiol group of L-cysteine is in a reduced SH form (hereafter, it may be referred to as “GSH”) or a glutathione oxidized form in which the thiol groups of L-cysteine are oxidized to form a disulfide bond between 2 glutathione molecules (hereafter, it may be referred to as “GSSG”).

Examples of known methods for producing glutathione include a method for producing glutathione by fermentation using yeast (Patent Document 1) and a method for producing glutathione comprising producing γ-glutamylcysteine synthetase or glutathione synthetase using microorganisms and enzymatically ligating L-glutamic acid, L-cysteine, and glycine (Patent Documents 2 and 3).

Patent Document 4 discloses a method for producing glutathione or γ-glutamylcysteine comprising culturing microorganisms with activity of proteins having glutathione transport activity and activity of proteins involved in biosynthesis of glutathione or γ-glutamylcysteine higher than those of parent strains in a medium to produce and accumulate glutathione or γ-glutamylcysteine in the medium and collecting glutathione or γ-glutamylcysteine from the culture product. Patent Document 4 describes, in Example 4, that Escherichea coli strains overexpressing the E. coli-derived glutamate-cysteine ligase gshA gene and the glutathione synthetase gshB gene were cultured and the glutathione concentration in the medium was 160 mg/l.

Non-Patent Document 1 describes a method for producing glutathione comprising culturing E. coli strains transformed with an expression vector comprising the bifunctional glutathione synthetase gshF gene under the control of a constitutive promoter in a medium supplemented with constitutive amino acids of glutathione; i.e., L-cysteine, L-glutamic acid, and glycine.

### PRIOR ART DOCUMENTS

### Patent Documents

Patent Document 1: WO 2016/140349
Patent Document 2: JP S60-27396 A (1985)
Patent Document 3: JP S60-27397 A (1985)
Patent Document 4: WO 2008/126784
Patent Document 5: JP Patent No. 6,018,357

### Non-Patent Documents

Non-Patent Document 1: Journal of Biotechnology, 2018, https://doi.org/10.1016/j.jbiotec.2018.11.001
Non-Patent Document 2: J. Biotechnol., 289, 39, 2019

### SUMMARY OF THE INVENTION

### Objects to Be Attained by the Invention

Non-Patent Document 2 is known as another example of glutathione fermentation performed with the use of microorganisms such as bacteria; however, practical application thereof is difficult because of the addition of an expensive substrate; i.e., L-cysteine, to a culture solution. While Patent Document 5 describes that an example of bacteria-induced glutathione production by fermentation without the addition of L-cysteine, wherein glutathione productivity was low (< 1 g/l), and practicability thereof was poor. Accordingly, an improvement is desired for such technique.

The present invention is intended to improve productivity of glutathione and substances associated therewith; that is, γ-glutamylcysteine, bis-γ-glutamylcystine, γ-glutamylcystine, reduced glutathione, and/or oxidized glutathione, by fermentation of microorganisms such as bacteria.

### Means for Attaining the Objects

The present inventors have conducted concentrated studies in order to attain the above objects. As a result,the present inventors discovered that productivity of glutathione and substances associated therewith would be improved to a significant extent in microorganisms in which the expression level of a gene encoding serine-O-acetyltransferase (EC:2.3.1.30) is enhanced. This has led to the completion of the present invention.

Specifically, the present invention includes the following.
[I] A microbial strain capable of overproduction of γ-glutamylcysteine, bis-γ-glutamylcystine, γ-glutamylcystine, reduced glutathione, and/or oxidized glutathione, which has the gene modification :
enhanced expression of a gene encoding serine-O-acetyltransferase (EC:2.3.1.30). [II] The microbial strain according to [I], which has the gene modification [2] and at least one gene modification selected from among the gene modifications [3] and [4]:
disruption of a gene encoding γ-glutamyltransferase (EC:3.4.19.13); and
enhanced expression of a gene encoding glutamate-cysteine ligase (EC:6.3.2.2) and/or a gene encoding glutathione synthetase (EC:6.3.2.3); and/or
enhanced expression of a gene encoding bifunctional glutathione synthetase.

[III] The microbial strain according to [I] or [II] comprising at least one gene modification selected from among the gene modifications [5], [6], [7], [8], and [9]:
disruption of a gene encoding tryptophanase (EC:4.1.99.1);
disruption of a gene encoding tripeptide peptidase (EC:3.4.11.4);
disruption of at least 1 gene encoding a protein involved in glutathione import;
disruption of a gene encoding glutathione reductase (EC:1.8.1.7); and
enhanced expression of at least 1 gene encoding a protein involved in putrescine export.

[IV] The microbial strain according to any of [I] to [III], which is a transformed bacterium.

[V] The microbial strain according to [IV], which is a transformed enteric bacterium

[VI] The microbial strain according to [IV], which is a transformed Gram-negative bacterium

[VII] The microbial strain according to [IV], which is a transformed E. coli strain.

[VIII] A method for producing γ-glutamylcysteine, bis-γ-glutamylcystine, γ-glutamylcystine, reduced glutathione, and/or oxidized glutathione comprising culturing the microbial strain according to any one of [I] to [VII].

This description includes part or all of the content as disclosed in the description and/or drawings of Japanese Patent Application No. 2020-079058, which is a priority document of the present disclosure.

### Effects of the Invention

The microbial strain according to one or more embodiments of the present invention is capable of efficient production of γ-glutamylcysteine, bis-γ-glutamylcystine, γ-glutamylcystine, reduced glutathione, and/or oxidized glutathione by fermentation.

The method for producing glutathione according to one or more embodiments of the present invention enables efficient production of the target substance.

### EMBODIMENTS OF THE INVENTION

### Host Microorganisms

Microbial strains serving as host strains (parent strains) of the microbial strains comprising particular gene modifications according to one or more embodiments of the present invention are preferably prokaryotic microorganisms and more preferably bacteria. The bacteria may be enteric bacteria. The bacteria may be Gram-negative bacteria, such as Escherichia sp. or Pantoea sp., or Gram-positive bacteria, such as Bacillus sp., Brevibacterium sp., or Corynebacterium sp., with Gram-negative bacteria being preferable and E. coli being particularly preferable.

The microbial strain according to one or more embodiments of the present invention can be a transformant derived from a host strain by disruption of a particular gene while carrying a particular gene.

### 1. Serine-O-Acetyltransferase

Serine-O-acetyltransferase (EC:2.3.1.30) is an enzyme that catalyzes a reaction of acetylating L-serine in a CoA-dependent manner to generate O-acetylcysteine. The origin, the structure, and other properties thereof are not particularly limited, as long as such enzyme has the activity described above.

The origin of serine-O-acetyltransferase is not particularly limited, and serine-O-acetyltransferase derived from microorganisms, animals, plants, and the like can be used. Serine-O-acetyltransferase derived from microorganisms is preferable, and serine-O-acetyltransferase derived from enteric bacteria such as Escherichia coli, bacteria such as coryneform bacteria, eukaryotic microorganisms such as yeasts, or the like is particularly preferable.

Specific examples of the nucleotide sequence of E. coli-derived serine-O-acetyltransferase and the amino acid sequence encoded by the nucleotide sequence are shown in SEQ ID NO: 21 and SEQ ID NO: 22, respectively.

Serine-O-acetyltransferase is not limited to the serine-O-acetyltransferase consisting of the amino acid sequence as shown in SEQ ID NO: 22. Other polypeptides having serine-O-acetyltransferase activity, such as active mutants of the serine-O-acetyltransferase or orthologs of different species, may be used. Other polypeptides having serine-O-acetyltransferase activity preferably exhibit activity of 10% or higher, more preferably 40% or higher, more preferably 60% or higher, more preferably 80% or higher, and further preferably 90% or higher, compared with the activity when serine-O-acetyltransferase consisting of the amino acid sequence as shown in SEQ ID NO: 22 is used under the activity assay conditions described above.

Specific examples of serine-O-acetyltransferase include:
(1A) a polypeptide consisting of the amino acid sequence as shown in SEQ ID NO: 22;
(1B) a polypeptide consisting of an amino acid sequence derived from the amino acid sequence as shown in SEQ ID NO: 22 by addition, deletion, or substitution of 1 or a plurality of amino acids (which is a polypeptide consisting of an amino acid sequence derived from the amino acid sequence as shown in SEQ ID NO: 22 preferably by substitution, deletion, and/or addition, and more preferably by deletion and/or addition, of 1 or a plurality of amino acids at either or both of the N terminus and the C terminus) and having serine-O-acetyltransferase activity;
(1C) a polypeptide consisting of an amino acid sequence having 80% or higher, preferably 85% or higher, and more preferably 90% or higher, 95% or higher, 97% or higher, 98% or higher, or 99% or higher sequence identity to the amino acid sequence as shown in SEQ ID NO: 22 and having serine-O-acetyltransferase activity; and
(1D) a fragment of any of the polypeptides (1A) to (1C) having serine-O-acetyltransferase activity.

The fragment (1D) can be a polypeptide comprising preferably 200 or more, and more preferably 250 or more amino acids.

In (1B) above, the term “a plurality of” refers to, for example, 2 to 20, 2 to 15, 2 to 10, 2 to 7, 2 to 5, 2 to 4, or 2 or 3. In (1B) above and (2B), (3-1B), (3-2B), (3-3B), (3-4B), (4B), (5B), (6B), (7-1B), (7-2B), (7-3B), (7-4B), (8B), (9-1B), (9-2B), (9-3B), (9-4B), and (9-5B) below, the expression “addition, deletion, or substitution of 1 or a plurality of amino acids” indicates that 1 to a plurality of amino acids are subjected to addition, deletion, or substitution in total. Amino acid substitution is preferably conservative amino acid substitution. The term “conservative amino acid substitution” refers to substitution between amino acids having similar properties in terms of, for example, electric charge, side chains, polarity, and aromaticity. Amino acids having similar properties can be classified into: for example, basic amino acids, such as arginine, lysine, and histidine; acidic amino acids, such as aspartic acid and glutamic acid; uncharged polar amino acids, such as glycine, asparagine, glutamine, serine, threonine, cysteine, and tyrosine; nonpolar amino acids, such as leucine, isoleucine, alanine, valine, proline, phenylalanine, tryptophan, and methionine; branched amino acids, such as leucine, valine, and isoleucine; and aromatic amino acids, such as phenylalanine, tyrosine, tryptophan, and histidine. Hereafter, the term “conservative amino acid substitution” is used in the same sense.

In (1C) above, “sequence identity” is a value determined by aligning 2 amino acid sequences, introducing gaps, according to need, so as to maximize the extent of amino acid consistency therebetween, and determining a percentage (%) of identical amino acids based on the total number of amino acids in the amino acid sequence as shown in SEQ ID NO: 22. The “sequence identity” can be determined with the use of protein search systems, such as BLAST or FASTA (Karlin, S. et al., 1993, Proc. Natl. Acad. Sci., U.S.A., 90: 5873-5877; Altschul, S. F. et al., 1990, J. Mol. Biol., 215: 403-410; Pearson, W. R. et al., 1988, Proc. Natl. Acad. Sci., U.S.A., 85: 2444-2448). Hereafter, the “sequence identity” of amino acid sequences is used in the same sense.

The term “a gene encoding serine-O-acetyltransferase (EC:2.3.1.30)” refers to a nucleic acid (DNA or RNA, with DNA being preferable) encoding the amino acid sequence of serine-O-acetyltransferase.

SEQ ID NO: 21 shows an example of DNA encoding the amino acid sequence of E. coli-derived serine-O-acetyltransferase as shown in SEQ ID NO: 22. The nucleotide sequence of the nucleic acid encoding the amino acid sequence of serine-O-acetyltransferase may be codon-optimized for the host. It should be noted that the nucleotide sequence as shown in SEQ ID NO: 21 is not always present in that state in the genomic DNA of the microbial strain. The nucleotide sequence as shown in SEQ ID NO: 21 may be an exon sequence comprising one or more intron sequences therein.

Specific examples of nucleotide sequences of genes encoding the amino acid sequence of serine-O-acetyltransferase include the following.

(1E) The nucleotide sequence as shown in SEQ ID NO: 21;
(1F) a nucleotide sequence derived from the nucleotide sequence as shown in SEQ ID NO: 21 by addition, deletion, or substitution of 1 or a plurality of nucleotides (which is a nucleotide sequence derived from the nucleotide sequence as shown in SEQ ID NO: 21 preferably by substitution, deletion, and/or addition, and more preferably by deletion and/or addition, of 1 or a plurality of nucleotides at either or both of the 5′ terminus and the 3′ terminus) and encoding a polypeptide having serine-O-acetyltransferase activity;
(1G) a nucleotide sequence having 80% or higher, preferably 85% or higher, and more preferably 90% or higher, 95% or higher, 97% or higher, 98% or higher, or 99% or higher sequence identity to the nucleotide sequence as shown in SEQ ID NO: 21 and encoding a polypeptide having serine-O-acetyltransferase activity;
(1H) a partial nucleotide sequence of any of the nucleotide sequences (1E) to (1G) encoding an amino acid sequence of a polypeptide having serine-O-acetyltransferase activity;
(1I) a nucleotide sequence derived from any of the nucleotide sequences (1E) to (1H) by introduction of silent mutation (nucleotide substitution that does not alter amino acids to encode);
(1J) a nucleotide sequence encoding the amino acid sequence of any of the polypeptides (1A) to (1D); and
(1K) a nucleotide sequence comprising, as an exon sequence, any of the nucleotide sequences (1E) to (1J) and one or more intron sequences therein.

In (1G) above, “sequence identity” is a value determined by aligning 2 nucleotide sequences, introducing gaps, according to need, so as to maximize the extent of nucleotide consistency therebetween, and determining a percentage (%) of identical nucleotides based on the total number of nucleotides in the nucleotide sequence as shown in SEQ ID NO: 21. The “sequence identity” can be determined with the use of nucleotide sequence search systems, such as BLAST or FASTA (Karlin, S. et al., 1993, Proc. Natl. Acad. Sci., U.S.A., 90: 5873-5877; Altschul, S. F.et al., 1990, J. Mol. Biol., 215: 403-410; Pearson, W. R. et al., 1988, Proc. Natl. Acad. Sci., U.S.A., 85: 2444-2448). Hereafter, the “sequence identity” of nucleotide sequences is used in the same sense.

In (1F) above, the term “a plurality of” refers to, for example, 2 to 60, 2 to 45, 2 to 30, 2 to 21, 2 to 15, 2 to 6, or 2 or 3. In (1F) above and (2F), (3-1F), (3-2F), (3-3F), (3-4F), (4F), (5F), (6F), (7-1F), (7-2F), (7-3F), (7-4F), (8F), (9-1F), (9-2F), (9-3F), (9-4F), and (9-5F) below, the expression “addition, deletion, or substitution of 1 or a plurality of nucleotides” indicates that 1 to a plurality of nucleotides are subjected to addition, deletion, or substitution in total.

### 2. γ-Glutamyltransferase

γ-Glutamyltransferase (EC:3.4.19.13 or 2.3.2.2) is an enzyme that hydrolyzes γ-glutamylpeptide, such as glutathione.

Specific examples of γ-glutamyltransferase include:
(2A) a polypeptide consisting of the amino acid sequence as shown in SEQ ID NO: 24;
(2B) a polypeptide consisting of an amino acid sequence derived from the amino acid sequence as shown in SEQ ID NO: 24 by addition, deletion, or substitution of 1 or a plurality of amino acids (which is a polypeptide consisting of an amino acid sequence derived from the amino acid sequence as shown in SEQ ID NO: 24 preferably by substitution, deletion, and/or addition, and more preferably by deletion and/or addition, of 1 or a plurality of amino acids at either or both of the N terminus and the C terminus) and having γ-glutamyltransferase activity;
(2C) a polypeptide consisting of an amino acid sequence having 80% or higher, preferably 85% or higher, and more preferably 90% or higher, 95% or higher, 97% or higher, 98% or higher, or 99% or higher sequence identity to the amino acid sequence as shown in SEQ ID NO: 24 and having γ-glutamyltransferase activity; and
(2D) a fragment of any of the polypeptides (2A) to (2C) having γ-glutamyltransferase activity.

The fragment (2D) can be a polypeptide comprising preferably 200 or more, more preferably 300 or more, more preferably 400 or more, more preferably 500 or more, and more preferably 550 or more amino acids.

In (2B) above, the term “a plurality of” refers to, for example, 2 to 20, 2 to 15, 2 to 10, 2 to 7, 2 to 5, 2 to 4, or 2 or 3. Amino acid substitution is preferably conservative amino acid substitution.

In (2C) above, “sequence identity” is a value determined by aligning 2 amino acid sequences, introducing gaps, according to need, so as to maximize the extent of amino acid consistency therebetween, and determining a percentage (%) of identical amino acids based on the total number of amino acids in the amino acid sequence as shown in SEQ ID NO: 24.

The term “a gene encoding γ-glutamyltransferase” refers to a nucleic acid (DNA or RNA, with DNA being preferable) encoding the amino acid sequence of γ-glutamyltransferase, and such gene is included in the genomic DNA in the chromosome of the wild-type microorganism before disruption of γ-glutamyltransferase therein.

SEQ ID NO: 23 shows an example of DNA encoding the amino acid sequence of E. coli-derived γ-glutamyltransferase as shown in SEQ ID NO: 24. It should be noted that the nucleotide sequence as shown in SEQ ID NO: 23 is not always present in that state in the genomic DNA of the wild-type microorganism. The nucleotide sequence as shown in SEQ ID NO: 23 may be an exon sequence comprising one or more intron sequences therein.

Specific examples of nucleotide sequences of genes encoding the amino acid sequence of γ-glutamyltransferase include:
(2E) the nucleotide sequence as shown in SEQ ID NO: 23;
(2F) a nucleotide sequence derived from the nucleotide sequence as shown in SEQ ID NO: 23 by addition, deletion, or substitution of 1 or a plurality of nucleotides (which is a nucleotide sequence derived from the nucleotide sequence as shown in SEQ ID NO: 23 preferably by substitution, deletion, and/or addition, and more preferably by deletion and/or addition, of 1 or a plurality of nucleotides at either or both of the 5′ terminus and the 3′ terminus) and encoding a polypeptide having γ-glutamyltransferase activity;
(2G) a nucleotide sequence having 80% or higher, preferably 85% or higher, and more preferably 90% or higher, 95% or higher, 97% or higher, 98% or higher, or 99% or higher sequence identity to the nucleotide sequence as shown in SEQ ID NO: 23 and encoding a polypeptide having γ-glutamyltransferase activity;
(2H) a partial nucleotide sequence of any of the nucleotide sequences (2E) to (2G) encoding an amino acid sequence of a polypeptide having γ-glutamyltransferase activity;
(2I) a nucleotide sequence derived from any of the nucleotide sequences (2E) to (2H) by introduction of silent mutation (nucleotide substitution that does not alter amino acids to encode);
(2J) a nucleotide sequence encoding the amino acid sequence of any of the polypeptides (2A) to (2D); and
(2 K) a nucleotide sequence comprising, as an exon sequence, any of the nucleotide sequences (2E) to (2J) and one or more intron sequences therein.

In (2F) above, the term “a plurality of” refers to, for example, 2 to 60, 2 to 45, 2 to 30, 2 to 21, 2 to 15, 2 to 6, or 2 or 3.

In (2G) above, “sequence identity” is a value determined by aligning 2 nucleotide sequences, introducing gaps, according to need, so as to maximize the extent of nucleotide consistency therebetween, and determining a percentage (%) of identical nucleotides based on the total number of nucleotides in the nucleotide sequence as shown in SEQ ID NO: 23.

### 3-1. Glutamate-Cysteine Ligase

Glutamate-cysteine ligase (EC:6.3.2.2) is an enzyme that catalyzes a reaction of recognizing L-cysteine (L-Cys) as a substrate in the presence of ATP and allowing L-cysteine to bind to L-glutamic acid (L-Glu) to generate γ-Glu-Cys. Such enzyme is not particularly limited in terms of the origin, the structure, and other properties, provided that it has the activity described above. Such activity is referred to as “glutamate-cysteine ligase activity” herein. At 1 U of the activity, 1 µmol of γ-glutamylcysteine is generated at 30° C. in 1 minute, and such activity is assayed under the conditions described below.

### Assay Conditions

An enzyme solution is added to a 50 mM Tris-HCl buffer solution (pH 8.0) containing 10 mM ATP, 15 mM L-glutamic acid, 15 mM L-cysteine, and 10 mM magnesium sulfate, the reaction is allowed to proceed while maintaining the temperature at 30° C., and the reaction is terminated with the addition of 6 N hydrochloric acid. By performing high-performance liquid chromatography, γ-glutamylcysteine in the reaction solution is quantified.

The conditions for the high-performance liquid chromatography are as described below. Under the conditions described below, glutathione (GSH), γ-glutamylcysteine (γ-GC), bis-γ-glutamylcystine (reduced γ-GC), and oxidized glutathione (GSSG) are eluted in that order.

### HPLC Conditions

Column: ODS-HG-3 (4.6 mm φ × 150 mm, Nomura Chemical Co., Lid.)
Eluate: A solution prepared by dissolving 12.2 g of potassium dihydrogen-phosphate and 3.6 g of sodium heptane sulfonate in 1.8 l of distilled water, adjusting pH at 2.8 with the aid of phosphoric acid, and adding 186 ml of methanol
Flow rate: 1.0 ml/min
Column temperature: 40° C.
Assay wavelength: 210 nm

Use of glutamate-cysteine ligase having glutamate-cysteine ligase activity of 0.5 U or higher per 1 mg of a protein (i.e., specific activity) is preferable.

The origin of glutamate-cysteine ligase is not particularly limited, and glutamate-cysteine ligase derived from microorganisms, animals, plants, and the like can be used. Glutamate-cysteine ligase derived from microorganisms is preferable, and glutamate-cysteine ligase derived from enteric bacteria such as Escherichia coli, bacteria such as coryneform bacteria, eukaryotic microorganisms such as yeasts, or the like is particularly preferable.

Specific examples of the nucleotide sequence of E. coli-derived glutamate-cysteine ligase and the amino acid sequence encoded by the nucleotide sequence are shown in SEQ ID NO: 55 and SEQ ID NO: 56, respectively.

Glutamate-cysteine ligase is not limited to the glutamate-cysteine ligase consisting of the amino acid sequence as shown in SEQ ID NO: 56. Other polypeptides having glutamate-cysteine ligase activity, such as active mutants of the glutamate-cysteine ligase or orthologs of different species, may be used. Other polypeptides having glutamate-cysteine ligase activity preferably exhibit activity of 10% or higher, more preferably 40% or higher, more preferably 60% or higher, more preferably 80% or higher, and further preferably 90% or higher, compared with the activity when glutamate-cysteine ligase consisting of the amino acid sequence as shown in SEQ ID NO: 56 is used under the activity assay conditions described above.

Specific examples of glutamate-cysteine ligase include:
(3-1A) a polypeptide consisting of the amino acid sequence as shown in SEQ ID NO: 56;
(3-1B) a polypeptide consisting of an amino acid sequence derived from the amino acid sequence as shown in SEQ ID NO: 56 by addition, deletion, or substitution of 1 or a plurality of amino acids (which is a polypeptide consisting of an amino acid sequence derived from the amino acid sequence as shown in SEQ ID NO: 56 preferably by substitution, deletion, and/or addition, and more preferably by deletion and/or addition, of 1 or a plurality of amino acids at either or both of the N terminus and the C terminus) and having glutamate-cysteine ligase activity;
(3-1C) a polypeptide consisting of an amino acid sequence having 80% or higher, preferably 85% or higher, and more preferably 90% or higher, 95% or higher, 97% or higher, 98% or higher, or 99% or higher sequence identity to the amino acid sequence as shown in SEQ ID NO: 56 and having glutamate-cysteine ligase activity; and
(3-1D) a fragment of any of the polypeptides (3-1A) to (3-1C) having glutamate-cysteine ligase activity.

The fragment (3-1D) can be a polypeptide comprising preferably 200 or more, more preferably 300 or more, more preferably 400 or more, more preferably 450 or more, and more preferably 500 or more amino acids.

In (3-1B) above, the term “a plurality of” refers to, for example, 2 to 20, 2 to 15, 2 to 10, 2 to 7, 2 to 5, 2 to 4, or 2 or 3. Amino acid substitution is preferably conservative amino acid substitution.

In (3-1C) above, “sequence identity” is a value determined by aligning 2 amino acid sequences, introducing gaps, according to need, so as to maximize the extent of amino acid consistency therebetween, and determining a percentage (%) of identical amino acids based on the total number of amino acids in the amino acid sequence as shown in SEQ ID NO: 56.

The term “a gene encoding glutamate-cysteine ligase” refers to a nucleic acid (DNA or RNA, with DNA being preferable) encoding the amino acid sequence of glutamate-cysteine ligase.

SEQ ID NO: 55 shows an example of DNA encoding the amino acid sequence of E. coli-derived glutamate-cysteine ligase as shown in SEQ ID NO: 56. The nucleotide sequence of the nucleic acid encoding the amino acid sequence of glutamate-cysteine ligase may be codon-optimized for the host.

Specific examples of nucleotide sequences of genes encoding the amino acid sequence of glutamate-cysteine ligase include:
(3-1E) the nucleotide sequence as shown in SEQ ID NO: 55;
(3-1F) a nucleotide sequence derived from the nucleotide sequence as shown in SEQ ID NO: 55 by addition, deletion, or substitution of 1 or a plurality of nucleotides (which is a nucleotide sequence derived from the nucleotide sequence as shown in SEQ ID NO: 55 preferably by substitution, deletion, and/or addition, and more preferably by deletion and/or addition, of 1 or a plurality of nucleotides at either or both of the 5′ terminus and the 3′ terminus) and encoding a polypeptide having glutamate-cysteine ligase activity;
(3-1G) a nucleotide sequence having 80% or higher, preferably 85% or higher, and more preferably 90% or higher, 95% or higher, 97% or higher, 98% or higher, or 99% or higher sequence identity to the nucleotide sequence as shown in SEQ ID NO: 55 and encoding a polypeptide having glutamate-cysteine ligase activity;
(3-1H) a partial nucleotide sequence of any of the nucleotide sequences (3-1E) to (3-1G) encoding an amino acid sequence of a polypeptide having glutamate-cysteine ligase activity;
(3-1I) a nucleotide sequence derived from any of the nucleotide sequences (3-1E) to (3-1H) by introduction of silent mutation (nucleotide substitution that does not alter amino acids to encode);
(3-1J) a nucleotide sequence encoding the amino acid sequence of any of the polypeptides (3-1A) to (3-1D); and
(3-1K) a nucleotide sequence comprising, as an exon sequence, any of the nucleotide sequences (3-1E) to (3-1J) and one or more intron sequences therein.

In (3-1F) above, the term “a plurality of” refers to, for example, 2 to 60, 2 to 45, 2 to 30, 2 to 21, 2 to 15, 2 to 6, or 2 or 3.

In (3-1G) above, “sequence identity” is a value determined by aligning 2 nucleotide sequences, introducing gaps, according to need, so as to maximize the extent of nucleotide consistency therebetween, and determining a percentage (%) of identical nucleotides based on the total number of nucleotides in the nucleotide sequence as shown in SEQ ID NO: 55.

### 3-2. Glutathione Synthetase

Glutathione synthetase (EC:6.3.2.3) is an enzyme that catalyzes a reaction of recognizing γ-Glu-Cys as a substrate in the presence of ATP and allowing γ-Glu-Cys to bind to glycine (Gly) to generate γ-Glu-Cys-Gly. Such enzyme is not particularly limited in terms of the origin, the structure, and other properties, provided that it has the activity described above. Such activity is referred to as “glutathione synthetase activity” herein. At 1 U of the activity, 1 µmol of glutathione is generated at 30° C. in 1 minute, and such activity is assayed under the conditions described below.

### Assay Conditions

An enzyme solution is added to a 50 mM Tris-HCl buffer solution (pH 8.0) containing 10 mM ATP, 15 mM γ-glutamylcysteine, 15 mM glycine, and 10 mM magnesium sulfate, the reaction is allowed to proceed while maintaining the temperature at 30° C., and the reaction is terminated with the addition of 6 N hydrochloric acid. By performing high-performance liquid chromatography, glutathione in the reaction solution is quantified.

The conditions for the high-performance liquid chromatography are as described above with regard to the method of glutamate-cysteine ligase activity assay.

Use of glutathione synthetase having glutathione synthetase activity of 0.5 U or higher per 1 mg of a protein (i.e., specific activity) is preferable.

The origin of glutathione synthetase is not particularly limited, and glutathione synthetase derived from microorganisms, animals, plants, and the like can be used. Glutathione synthetase derived from microorganisms is preferable, and glutathione synthetase derived from enteric bacteria such as Escherichia coli, bacteria such as coryneform bacteria, eukaryotic microorganisms such as yeasts, microorganisms of Hydrogenophilales, or the like is particularly preferable.

Glutathione synthetase derived from microorganisms of Hydrogenophilales is preferably glutathione synthetase derived from microorganisms of Thiobacillus, and more preferably glutathione synthetase derived from microorganisms of Thiobacillus denitrificans. Glutathione synthetase derived from the Thiobacillus denitrificans strain ATCC 25259 is particularly preferable.

### Preferable Embodiments of E. Coli-Derived Glutathione Synthetase or Mutant Thereof

Specific examples of the nucleotide sequence of E. coli-derived glutathione synthetase and the amino acid sequence encoded by the nucleotide sequence are shown in SEQ ID NO: 57 and SEQ ID NO: 58, respectively.

Glutathione synthetase is not limited to the glutathione synthetase consisting of the amino acid sequence as shown in SEQ ID NO: 58. Other polypeptides having glutathione synthetase activity, such as active mutants of the glutathione synthetase or orthologs of different species, may be used. Other polypeptides having glutathione synthetase activity preferably exhibit activity of 10% or higher, more preferably 40% or higher, more preferably 60% or higher, more preferably 80% or higher, and further preferably 90% or higher, compared with the activity when glutathione synthetase consisting of the amino acid sequence as shown in SEQ ID NO: 58 is used under the activity assay conditions described above.

Specific examples of E. coli-derived glutathione synthetase or a mutant thereof include:
(3-2A) a polypeptide consisting of the amino acid sequence as shown in SEQ ID NO: 58;
(3-2B) a polypeptide consisting of an amino acid sequence derived from the amino acid sequence as shown in SEQ ID NO: 58 by addition, deletion, or substitution of 1 or a plurality of amino acids (which is a polypeptide consisting of an amino acid sequence derived from the amino acid sequence as shown in SEQ ID NO: 58 preferably by substitution, deletion, and/or addition, and more preferably by deletion and/or addition, of 1 or a plurality of amino acids at either or both of the N terminus and the C terminus) and having glutathione synthetase activity;
(3-2C) a polypeptide consisting of an amino acid sequence having 80% or higher, preferably 85% or higher, and more preferably 90% or higher, 95% or higher, 97% or higher, 98% or higher, or 99% or higher sequence identity to the amino acid sequence as shown in SEQ ID NO: 58 and having glutathione synthetase activity; and
(3-2D) a fragment of any of the polypeptides (3-2A) to (3-2C) having glutathione synthetase activity.

The fragment (3-2D) can be a polypeptide comprising preferably 200 or more, more preferably 250 or more, and more preferably 300 or more amino acids.

In (3-2B) above, the term “a plurality of” refers to, for example, 2 to 20, 2 to 15, 2 to 10, 2 to 7, 2 to 5, 2 to 4, or 2 or 3. Amino acid substitution is preferably conservative amino acid substitution.

In (3-2C) above, “sequence identity” is a value determined by aligning 2 amino acid sequences, introducing gaps, according to need, so as to maximize the extent of amino acid consistency therebetween, and determining a percentage (%) of identical amino acids based on the total number of amino acids in the amino acid sequence as shown in SEQ ID NO: 58.

The term “a gene encoding glutathione synthetase” refers to a nucleic acid (DNA or RNA, with DNA being preferable) encoding the amino acid sequence of glutathione synthetase.

SEQ ID NO: 57 shows an example of DNA encoding the amino acid sequence of E. coli-derived glutathione synthetase as shown in SEQ ID NO: 58. The nucleotide sequence of the nucleic acid encoding the amino acid sequence of glutathione synthetase may be codon-optimized for the host.

Specific examples of nucleotide sequences of genes encoding the amino acid sequence of the E. coli-derived glutathione synthetase or a mutant thereof include:
(3-2E) the nucleotide sequence as shown in SEQ ID NO: 57;
(3-2F) a nucleotide sequence derived from the nucleotide sequence as shown in SEQ ID NO: 57 by addition, deletion, or substitution of 1 or a plurality of nucleotides (which is a nucleotide sequence derived from the nucleotide sequence as shown in SEQ ID NO: 57 preferably by substitution, deletion, and/or addition, and more preferably by deletion and/or addition, of 1 or a plurality of nucleotides at either or both of the 5′ terminus and the 3′ terminus) and encoding a polypeptide having glutathione synthetase activity;
(3-2G) a nucleotide sequence having 80% or higher, preferably 85% or higher, and more preferably 90% or higher, 95% or higher, 97% or higher, 98% or higher, or 99% or higher sequence identity to the nucleotide sequence as shown in SEQ ID NO: 57 and encoding a polypeptide having glutathione synthetase activity;
(3-2H) a partial nucleotide sequence of any of the nucleotide sequences (3-2E) to (3-2G) encoding an amino acid sequence of a polypeptide having glutathione synthetase activity;
(3-2I) a nucleotide sequence derived from any of the nucleotide sequences (3-2E) to (3-2H) by introduction of silent mutation (nucleotide substitution that does not alter amino acids to encode);
(3-2J) a nucleotide sequence encoding the amino acid sequence of any of the polypeptides (3-2A) to (3-2D); and
(3-2K) a nucleotide sequence comprising, as an exon sequence, any of the nucleotide sequences (3-2E) to (3-2J) and one or more intron sequences therein.

In (3-2F) above, the term “a plurality of” refers to, for example, 2 to 60, 2 to 45, 2 to 30, 2 to 21, 2 to 15, 2 to 6, or 2 or 3.

In (3-2G) above, “sequence identity” is a value determined by aligning 2 nucleotide sequences, introducing gaps, according to need, so as to maximize the extent of nucleotide consistency therebetween, and determining a percentage (%) of identical nucleotides based on the total number of nucleotides in the nucleotide sequence as shown in SEQ ID NO: 57.

### Preferable Embodiments of Thiobacillus Denitrificans-Derived Glutathione Synthetase or Mutant Thereof

Other preferable specific examples of glutathione synthetase include wild-type glutathione synthetase derived from the Thiobacillus denitrificans strain ATCC 25259 and active mutants thereof. Specific examples of the nucleotide sequence of the wild-type glutathione synthetase derived from the Thiobacillus denitrificans strain ATCC 25259 and the amino acid sequence encoded by the nucleotide sequence are shown in SEQ ID NO: 49 and SEQ ID NO: 50, respectively. An active mutant of the wild-type glutathione synthetase is a polypeptide preferably exhibiting activity of 10% or higher, more preferably 40% or higher, more preferably 60% or higher, more preferably 80% or higher, and further preferably 90% or higher, compared with the activity when the wild-type glutathione synthetase consisting of the amino acid sequence as shown in SEQ ID NO: 50 is used under the activity assay conditions described above.

Specific examples of glutathione synthetase derived from the Thiobacillus denitrificans strain ATCC 25259 or a mutant thereof include:
(3-3A) a polypeptide consisting of the amino acid sequence as shown in SEQ ID NO: 50;
(3-3B) a polypeptide consisting of an amino acid sequence derived from the amino acid sequence as shown in SEQ ID NO: 50 by addition, deletion, or substitution of 1 or a plurality of amino acids (which is a polypeptide consisting of an amino acid sequence derived from the amino acid sequence as shown in SEQ ID NO: 50 preferably by substitution, deletion, and/or addition, and more preferably by deletion and/or addition, of 1 or a plurality of amino acids at either or both of the N terminus and the C terminus) and having glutathione synthetase activity;
(3-3C) a polypeptide consisting of an amino acid sequence having 80% or higher, preferably 85% or higher, and more preferably 90% or higher, 95% or higher, 97% or higher, 98% or higher, or 99% or higher sequence identity to the amino acid sequence as shown in SEQ ID NO: 50 and having glutathione synthetase activity; and
(3-3D) a fragment of any of the polypeptides (3-3A) to (3-3C) having glutathione synthetase activity.

The fragment (3-3D) can be a polypeptide comprising preferably 200 or more, more preferably 250 or more, and more preferably 300 or more amino acids.

In (3-3B) above, the term “a plurality of” refers to, for example, 2 to 20, 2 to 15, 2 to 10, 2 to 7, 2 to 5, 2 to 4, or 2 or 3. Amino acid substitution is preferably conservative amino acid substitution.

In (3-3C) above, “sequence identity” is a value determined by aligning 2 amino acid sequences, introducing gaps, according to need, so as to maximize the extent of amino acid consistency therebetween, and determining a percentage (%) of identical amino acids based on the total number of amino acids in the amino acid sequence as shown in SEQ ID NO: 50.

SEQ ID NO: 49 shows an example of DNA encoding the amino acid sequence of glutathione synthetase derived from the Thiobacillus denitrificans strain ATCC 25259 as shown in SEQ ID NO: 50. The nucleotide sequence of the nucleic acid encoding the amino acid sequence of glutathione synthetase may be codon-optimized for the host.

Specific examples of nucleotide sequences of genes encoding the amino acid sequence of the glutathione synthetase derived from the Thiobacillus denitrificans strain ATCC 25259 or a mutant thereof include:
(3-3E) the nucleotide sequence as shown in SEQ ID NO: 49;
(3-3F) a nucleotide sequence derived from the nucleotide sequence as shown in SEQ ID NO: 49 by addition, deletion, or substitution of 1 or a plurality of nucleotides (which is a nucleotide sequence derived from the nucleotide sequence as shown in SEQ ID NO: 49 preferably by substitution, deletion, and/or addition, and more preferably by deletion and/or addition, of 1 or a plurality of nucleotides at either or both of the 5′ terminus and the 3′ terminus) and encoding a polypeptide having glutathione synthetase activity;
(3-3G) a nucleotide sequence having 80% or higher, preferably 85% or higher, and more preferably 90% or higher, 95% or higher, 97% or higher, 98% or higher, or 99% or higher sequence identity to the nucleotide sequence as shown in SEQ ID NO: 49 and encoding a polypeptide having glutathione synthetase activity;
(3-3H) a partial nucleotide sequence of any of the nucleotide sequences (3-3E) to (3-3G) encoding an amino acid sequence of a polypeptide having glutathione synthetase activity;
(3-3I) a nucleotide sequence derived from any of the nucleotide sequences (3-3E) to (3-3H) by introduction of silent mutation (nucleotide substitution that does not alter amino acids to encode);
(3-3J) a nucleotide sequence encoding the amino acid sequence of any of the polypeptides (3-3A) to (3-3D); and
(3-3 K) a nucleotide sequence comprising, as an exon sequence, any of the nucleotide sequences (3-3E) to (3-3J) and one or more intron sequences therein.

In (3-3F) above, the term “a plurality of” refers to, for example, 2 to 60, 2 to 45, 2 to 30, 2 to 21, 2 to 15, 2 to 6, or 2 or 3.

In (3-3G) above, “sequence identity” is a value determined by aligning 2 nucleotide sequences, introducing gaps, according to need, so as to maximize the extent of nucleotide consistency therebetween, and determining a percentage (%) of identical nucleotides based on the total number of nucleotides in the nucleotide sequence as shown in SEQ ID NO: 49.

### Preferable Embodiments of Active Mutants of Thiobacillus denitrificans-Derived Glutathione Synthetase

Another preferable example of glutathione synthetase is an active mutant of wild-type glutathione synthetase derived from the Thiobacillus denitrificans strain ATCC 25259 comprising the amino acid sequence as shown in SEQ ID NO: 50. The polypeptide described in WO 2018/084165 is particularly preferable.

Specific examples of the active mutants include:
(3-4A) a polypeptide consisting of an amino acid sequence 3-4A derived from the amino acid sequence as shown in SEQ ID NO: 50 by substitution of one or a plurality of amino acids selected from the group consisting of amino acids 13, 17, 20, 23, 39, 70, 78, 101, 113, 125, 126, 136, 138, 149, 152, 154, 155, 197, 200, 215, 226, 227, 230, 239, 241, 246, 249, 254, 260, 262, 263, 270, 278, 299, 305, 307, and 310;
(3-4B) a polypeptide consisting of an amino acid sequence derived from the amino acid sequence 3-4A by addition, deletion, or substitution of 1 or a plurality of amino acids other than the amino acids mentioned above (which is a polypeptide consisting of an amino acid sequence derived from the amino acid sequence 3-4A preferably by substitution, deletion, and/or addition, and more preferably by deletion and/or addition, of 1 or a plurality of amino acids at either or both of the N terminus and the C terminus) and having glutathione synthetase activity;
(3-4C) a polypeptide consisting of an amino acid sequence having consistency with the amino acid sequence 3-4A in the amino acid positions mentioned above and having 80% or higher, preferably 85% or higher, and more preferably 90% or higher, 95% or higher, 97% or higher, 98% or higher, or 99% or higher sequence identity to the amino acid sequence 3-4A in amino acid positions other than the amino acid positions mentioned above and having glutathione synthetase activity; and
(3-4D) a fragment of any of the polypeptides (3-4A) to (3-4C) having glutathione synthetase activity.

The fragment (3-4D) can be a polypeptide comprising preferably 150 or more, more preferably 200 or more, and more preferably 300 or more amino acids.

In (3-4B) above, the term “a plurality of” refers to, for example, 2 to 20, 2 to 15, 2 to 10, 2 to 7, 2 to 5, 2 to 4, or 2 or 3. Amino acid substitution is preferably conservative amino acid substitution.

In (3-4C) above, “sequence identity” is a value determined by aligning 2 amino acid sequences, introducing gaps, according to need, so as to maximize the extent of amino acid consistency therebetween, and determining a percentage (%) of identical amino acids based on the total number of amino acids in the amino acid sequence 3-4A.

It is more preferable that the amino acid sequence 3-4A be an amino acid sequence derived from the amino acid sequence as shown in SEQ ID NO: 50 by one or a plurality of amino acid substitutions selected from the group consisting of: serine at position 13, glutamic acid at position 17, threonine at position 20, leucine at position 23, threonine at position 39, serine at position 70, leucine at position 78, asparagine, glutamine, serine, or threonine at position 101, histidine at position 113, valine at position 125, asparagine at position 126, threonine at position 136, alanine at position 138, glutamine at position 149, glutamine at position 152, asparagine at position 154, leucine at position 155, glutamine at position 197, serine at position 200, aspartic acid at position 215, arginine at position 226, serine at position 227, proline at position 230, serine at position 239, histidine at position 241, arginine at position 246, glutamic acid at position 249, aspartic acid at position 254, alanine, cysteine, glycine, glutamine, or threonine at position 260, cysteine at position 262, arginine at position 263, isoleucine at position 270, glycine or alanine at position 278, alanine at position 299, glycine at position 305, valine at position 307, and threonine at position 310.

It is particularly preferable that the amino acid sequence 3-4A be an amino acid sequence derived from the amino acid sequence as shown in SEQ ID NO: 50 by any of amino acid substitutions (1) to (35) below:
(1) serine at position 13;
(2) glutamic acid at position 17, histidine at position 113, and proline at position 230;
(3) threonine at position 20 and aspartic acid at position 215;
(4) threonine at position 20 and histidine at position 241;
(5) leucine at position 23 and asparagine at position 126;
(6) threonine at position 39 and alanine at position 260;
(7) serine at position 70 and alanine at position 260;
(8) leucine at position 78 and alanine at position 278;
(9) asparagine at position 101;
(10) glutamine at position 101;
(11) serine at position 101;
(12) serine at position 101 and alanine at position 260;
(13) threonine at position 101;
(14) valine at position125 and glutamic acid at position 249;
(15) valine at position 125 and glutamine at position 152;
(16) threonine at position 136;
(17) alanine at position 138, glutamine at position 149, histidine at position 241, and glutamine at position 263;
(18) asparagine at position 154 and arginine at position 246;
(19) leucine at position 155 and serine at position 239;
(20) glutamine at position 197;
(21) serine at position 200 and alanine at position 260;
(22) arginine at position 226 and alanine at position 260;
(23) serine at position 227 and alanine at position 260;
(24) aspartic acid at position 254 and alanine at position 260;
(25) alanine at position 260;
(26) alanine at position 260, glycine at position 278, and valine at position 307;
(27) alanine at position 260 and alanine at position 299;
(28) alanine at position 260 and glycine at position 305;
(29) alanine at position 260 and threonine at position 310;
(30) cysteine at position 260;
(31) glycine at position 260;
(32) glutamine at position 260;
(33) threonine at position 260;
(34) cysteine at position 262; and
(35) isoleucine at position 270.

The nucleotide sequence encoding the amino acid sequence of any of the polypeptides (3-4A) to (3-4D) above can be used as the “gene encoding glutathione synthetase.”

SEQ ID NO: 51 shows an example of a nucleotide sequence encoding an amino acid sequence of an active mutant (SEQ ID NO: 52) derived from the amino acid sequence of glutathione synthetase derived from the Thiobacillus denitrificans strain ATCC 25259 as shown in SEQ ID NO: 50 with substitution of alanine for valine at position 260. The nucleotide sequence of the nucleic acid encoding the amino acid sequence of the active mutant of glutathione synthetase derived from the Thiobacillus denitrificans strain ATCC 25259 may be codon-optimized for the host. For example, SEQ ID NO: 51 shows the nucleotide sequence encoding the amino acid sequence as shown in SEQ ID NO: 52, which is codon-optimized for expression in E. coli.

### 4. Bifunctional Glutathione Synthetase

Bifunctional glutathione synthetase is an enzyme that has activity of catalyzing a reaction of recognizing L-Cys as a substrate in the presence of ATP and allowing L-Cys to bind to L-Glu to generate γ-Glu-Cys and activity of catalyzing a reaction of recognizing γ-Glu-Cys as a substrate in the presence of ATP and allowing γ-Glu-Cys to bind to Gly to generate γ-Glu-Cys-Gly. Such enzyme is not particularly limited in terms of the origin, the structure, and other properties, provided that it has the activities described above. Such activities are collectively referred to as “bifunctional glutathione synthetase activity” herein. At 1 U of the activity, 1 µmol of γ-Glu-Cys-Gly (glutathione) is generated at 30° C. in 1 minute, and such activity is assayed under the conditions described below.

### Assay Conditions

An enzyme solution is added to a 50 mM Tris-HCl buffer solution (pH 8.0) containing 10 mM ATP, 15 mM L-glutamic acid, 15 mM L-cysteine, 15 mM glycine, and 10 mM magnesium sulfate, the reaction is allowed to proceed while maintaining the temperature at 30° C., and the reaction is terminated with the addition of 6 N hydrochloric acid. By performing high-performance liquid chromatography, glutathione in the reaction solution is quantified.

The conditions for the high-performance liquid chromatography are as described above with regard to the method of glutamate-cysteine ligase activity assay.

Use of bifunctional glutathione synthetase having bifunctional glutathione synthetase activity of 0.5 U or higher per 1 mg of a protein (i.e., specific activity) is preferable.

The origin of bifunctional glutathione synthetase is not particularly limited, and bifunctional glutathione synthetase derived from microorganisms, animals, plants, and the like can be used. Bifunctional glutathione synthetase derived from microorganisms is preferable. Bifunctional glutathione synthetase derived from bacteria is particularly preferable. More specifically, bifunctional glutathione synthetase derived from at least one bacterial species selected from the group consisting of the bacteria described below is preferable: bacteria of the genus Streptococcus, such as Streptococcus agalactiae, Streptococcus mutans, Streptococcus suis, and Streptococcus thermophilus; bacteria of the genus Lactobacillus, such as Lactobacillus plantarum; bacteria of the genus Desulfotalea, such as Desulfotalea psychrophile; bacteria of the genus Clostridium, such as Clostridium perfringens; bacteria of the genus Listeria, such as Listeria innocua and Listeria monocytogenes; bacteria of the genus Enterococcus, such as Enterococcus faecalis and Enterococcus faecium; bacteria of the genus Pasteurella, such as Pasteurella multocida; bacteria of the genus Mannheimia, such as Mannheimia succiniciprodecens; and bacteria of the genus Haemophilus, such as Haemophilus somnus.

Specific examples of the nucleotide sequence of the bifunctional glutathione synthetase derived from Streptococcus agalactiae and the amino acid sequence encoded by the nucleotide sequence are shown in SEQ ID NO: 53 and SEQ ID NO: 54, respectively. The nucleotide sequence as shown in SEQ ID NO: 53 encodes the bifunctional glutathione synthetase derived from Streptococcus agalactiae consisting of the amino acid sequence as shown in SEQ ID NO: 54 and exhibits the frequency of codon usage adapted to E. coli.

Bifunctional glutathione synthetase is not limited to the bifunctional glutathione synthetase consisting of the amino acid sequence as shown in SEQ ID NO: 54. Other polypeptides having bifunctional glutathione synthetase activity, such as active mutants of the bifunctional glutathione synthetase or orthologs of different species, may be used. Other polypeptides having bifunctional glutathione synthetase activity preferably exhibit activity of 10% or higher, more preferably 40% or higher, more preferably 60% or higher, more preferably 80% or higher, and further preferably 90% or higher, compared with the activity when the bifunctional glutathione synthetase consisting of the amino acid sequence as shown in SEQ ID NO: 54 is used under the activity assay conditions described above.

Specific examples of bifunctional glutathione synthetase include:
(4A) a polypeptide consisting of the amino acid sequence as shown in SEQ ID NO: 54;
(4B) a polypeptide consisting of an amino acid sequence derived from the amino acid sequence as shown in SEQ ID NO: 54 by addition, deletion, or substitution of 1 or a plurality of amino acids (which is a polypeptide consisting of an amino acid sequence derived from the amino acid sequence as shown in SEQ ID NO: 54 preferably by substitution, deletion, and/or addition, and more preferably by deletion and/or addition, of 1 or a plurality of amino acids at either or both of the N terminus and the C terminus) and having bifunctional glutathione synthetase activity;
(4C) a polypeptide consisting of an amino acid sequence having 80% or higher, preferably 85% or higher, and more preferably 90% or higher, 95% or higher, 97% or higher, 98% or higher, or 99% or higher sequence identity to the amino acid sequence as shown in SEQ ID NO: 54 and having bifunctional glutathione synthetase activity; and
(4D) a fragment of any of the polypeptides (4A) to (4C) having glutathione synthetase activity.

The fragment in (4D) above can be a polypeptide comprising preferably 400 or more, more preferably 500 or more, more preferably 600 or more, more preferably 700 or more, and more preferably 730 or more amino acids.

In (4B) above, the term “a plurality of” refers to, for example, 2 to 20, 2 to 15, 2 to 10, 2 to 7, 2 to 5, 2 to 4, or 2 or 3. Amino acid substitution is preferably conservative amino acid substitution.

In (4C) above, “sequence identity” is a value determined by aligning 2 amino acid sequences, introducing gaps, according to need, so as to maximize the extent of amino acid consistency therebetween, and determining a percentage (%) of identical amino acids based on the total number of amino acids in the amino acid sequence as shown in SEQ ID NO: 54.

The term “a gene encoding bifunctional glutathione synthetase” refers to a nucleic acid (DNA or RNA, with DNA being preferable) encoding the amino acid sequence of bifunctional glutathione synthetase.

Specific examples of nucleotide sequences of genes encoding the amino acid sequence of the bifunctional glutathione synthetase include:
(4E) the nucleotide sequence as shown in SEQ ID NO: 53;
(4F) a nucleotide sequence derived from the nucleotide sequence as shown in SEQ ID NO: 53 by addition, deletion, or substitution of 1 or a plurality of nucleotides (which is a nucleotide sequence derived from the nucleotide sequence as shown in SEQ ID NO: 53 preferably by substitution, deletion, and/or addition, and more preferably by deletion and/or addition, of 1 or a plurality of nucleotides at either or both of the 5′ terminus and the 3′ terminus) and encoding a polypeptide having bifunctional glutathione synthetase activity;
(4G) a nucleotide sequence having 80% or higher, preferably 85% or higher, and more preferably 90% or higher, 95% or higher, 97% or higher, 98% or higher, or 99% or higher sequence identity to the nucleotide sequence as shown in SEQ ID NO: 53 and encoding a polypeptide having bifunctional glutathione synthetase activity;
(4H) a partial nucleotide sequence of any of the nucleotide sequences (4E) to (4G) encoding an amino acid sequence of a polypeptide having bifunctional glutathione synthetase activity;
(4I) a nucleotide sequence derived from any of the nucleotide sequences (4E) to (4H) by introduction of silent mutation (nucleotide substitution that does not alter amino acids to encode);
(4J) a nucleotide sequence encoding the amino acid sequence of any of the polypeptides (4A) to (4D); and
(4K) a nucleotide sequence comprising, as an exon sequence, any of the nucleotide sequences (4E) to (4J) and one or more intron sequences therein.

In (4F) above, the term “a plurality of” refers to, for example, 2 to 60, 2 to 45, 2 to 30, 2 to 21, 2 to 15, 2 to 6, or 2 or 3.

In (4G) above, “sequence identity” is a value determined by aligning 2 nucleotide sequences, introducing gaps, according to need, so as to maximize the extent of nucleotide consistency therebetween, and determining a percentage (%) of identical nucleotides based on the total number of nucleotides in the nucleotide sequence as shown in SEQ ID NO: 53.

### 5. Tryptophanase

Tryptophanase (EC:4.1.99.1) is an enzyme protein having activity of degrading cysteine.

An example of microbial tryptophanase is TnaA. A gene encoding the amino acid sequence of TnaA is tnaA.

The term “a gene encoding tryptophanase” refers to a nucleic acid (DNA or RNA, with DNA being preferable) encoding the amino acid sequence of tryptophanase, and such gene is included in the genomic DNA in the chromosome of the wild-type microbial strain before disruption of the gene therein.

The microbial strain according to one or more embodiments of the present invention described below preferably has disruption of the tnaA gene.

Specific examples of a TnaA protein include:
(5A) a polypeptide consisting of the amino acid sequence as shown in SEQ ID NO: 38;
(5B) a polypeptide consisting of an amino acid sequence derived from the amino acid sequence as shown in SEQ ID NO: 38 by addition, deletion, or substitution of 1 or a plurality of amino acids (which is a polypeptide consisting of an amino acid sequence derived from the amino acid sequence as shown in SEQ ID NO: 38 preferably by substitution, deletion, and/or addition, and more preferably by deletion and/or addition, of 1 or a plurality of amino acids at either or both of the N terminus and the C terminus) and having tryptophanase activity;
(5C) a polypeptide consisting of an amino acid sequence having 80% or higher, preferably 85% or higher, and more preferably 90% or higher, 95% or higher, 97% or higher, 98% or higher, or 99% or higher sequence identity to the amino acid sequence as shown in SEQ ID NO: 38 and having tryptophanase activity; and
(5D) a fragment of any of the polypeptides (5A) to (5C) having tryptophanase activity.

The fragment (5D) can comprise preferably 200 or more, more preferably 300 or more, more preferably 400 or more, and more preferably 450 or more amino acids.

In (5B) above, the term “a plurality of” refers to, for example, 2 to 20, 2 to 15, 2 to 10, 2 to 7, 2 to 5, 2 to 4, or 2 or 3. Amino acid substitution is preferably conservative amino acid substitution.

In (5C) above, “sequence identity” is a value determined by aligning 2 amino acid sequences, introducing gaps, according to need, so as to maximize the extent of amino acid consistency therebetween, and determining a percentage (%) of identical amino acids based on the total number of amino acids in the amino acid sequence as shown in SEQ ID NO: 38.

The term “tnaA gene” refers to a nucleic acid (DNA or RNA, with DNA being preferable) encoding the amino acid sequence of TnaA and such gene is included in the genomic DNA in the chromosome of the wild-type microbial strain before disruption of the gene therein.

SEQ ID NO: 37 shows an example of DNA encoding the amino acid sequence of E. coli-derived TnaA as shown in SEQ ID NO: 38. It should be noted that the nucleotide sequence as shown in SEQ ID NO: 37 is not always present in that state in the genomic DNA of the wild-type microbial strain. The nucleotide sequence as shown in SEQ ID NO: 37 may be an exon sequence comprising one or more intron sequences therein.

Specific examples of nucleotide sequences of genes encoding the amino acid sequence of TnaA and the tnaA gene include:
(5E) the nucleotide sequence as shown in SEQ ID NO: 37;
(5F) a nucleotide sequence derived from the nucleotide sequence as shown in SEQ ID NO: 37 by addition, deletion, or substitution of 1 or a plurality of nucleotides (which is a nucleotide sequence derived from the nucleotide sequence as shown in SEQ ID NO: 37 preferably by substitution, deletion, and/or addition, and more preferably by deletion and/or addition, of 1 or a plurality of nucleotides at either or both of the 5′ terminus and the 3′ terminus) and encoding a polypeptide having tryptophanase activity;
(5G) a nucleotide sequence having 80% or higher, preferably 85% or higher, and more preferably 90% or higher, 95% or higher, 97% or higher, 98% or higher, or 99% or higher sequence identity to the nucleotide sequence as shown in SEQ ID NO: 37 and encoding a polypeptide having tryptophanase activity;
(5H) a partial nucleotide sequence of any of the nucleotide sequences (5E) to (5G) encoding an amino acid sequence of a polypeptide having tryptophanase activity;
(5I) a nucleotide sequence derived from any of the nucleotide sequences (5E) to (5H) by introduction of silent mutation (-nucleotide substitution that does not alter amino acids to encode);
(5J) a nucleotide sequence encoding the amino acid sequence of any of the polypeptides (5A) to (5D); and
(5K) a nucleotide sequence comprising, as an exon sequence, any of the nucleotide sequences (5E) to (5J) and one or more intron sequences therein.

In (5F) above, the term “a plurality of” refers to, for example, 2 to 60, 2 to 45, 2 to 30, 2 to 21, 2 to 15, 2 to 6, or 2 or 3.

In (5G) above, “sequence identity” is a value determined by aligning 2 nucleotide sequences, introducing gaps, according to need, so as to maximize the extent of nucleotide consistency therebetween, and determining a percentage (%) of identical nucleotides based on the total number of nucleotides in the nucleotide sequence as shown in SEQ ID NO: 37.

### 6. Tripeptide Peptidase

Tripeptide peptidase (EC:3.4.11.4) is an enzyme that catalyzes a reaction of releasing the N-terminal amino acid residue from tripeptide.

Specific examples of tripeptide peptidase include:
(6A) a polypeptide consisting of the amino acid sequence as shown in SEQ ID NO: 26;
(6B) a polypeptide consisting of an amino acid sequence derived from the amino acid sequence as shown in SEQ ID NO: 26 by addition, deletion, or substitution of 1 or a plurality of amino acids (which is a polypeptide consisting of an amino acid sequence derived from the amino acid sequence as shown in SEQ ID NO: 26 preferably by substitution, deletion, and/or addition, and more preferably by deletion and/or addition, of 1 or a plurality of amino acids at either or both of the N terminus and the C terminus) and having tripeptide peptidase activity;
(6C) a polypeptide consisting of an amino acid sequence having 80% or higher, preferably 85% or higher, and more preferably 90% or higher, 95% or higher, 97% or higher, 98% or higher, or 99% or higher sequence identity to the amino acid sequence as shown in SEQ ID NO: 26 and having tripeptidase activity; and
(6D) a fragment of any of the polypeptides (6A) to (6C) having tripeptidase activity.

The fragment (6D) can be a polypeptide comprising preferably 200 or more, more preferably 300 or more, and more preferably 350 or more amino acids.

In (6B) above, the term “a plurality of” refers to, for example, 2 to 20, 2 to 15, 2 to 10, 2 to 7, 2 to 5, 2 to 4, or 2 or 3. Amino acid substitution is preferably conservative amino acid substitution.

In (6C) above, “sequence identity” is a value determined by aligning 2 amino acid sequences, introducing gaps, according to need, so as to maximize the extent of amino acid consistency therebetween, and determining a percentage (%) of identical amino acids based on the total number of amino acids in the amino acid sequence as shown in SEQ ID NO: 26.

The term “a gene encoding tripeptide peptidase” refers to a nucleic acid (DNA or RNA, with DNA being preferable) encoding the amino acid sequence of tripeptide peptidase, and such gene is included in the genomic DNA in the chromosome of the wild-type microorganism before disruption of tripeptide peptidase therein.

SEQ ID NO: 25 shows an example of DNA encoding the amino acid sequence of E. coli-derived tripeptide peptidase as shown in SEQ ID NO: 26. It should be noted that the nucleotide sequence as shown in SEQ ID NO: 25 is not always present in that state in the genomic DNA of the wild-type microorganism. The nucleotide sequence as shown in SEQ ID NO: 25 may be an exon sequence comprising one or more intron sequences therein.

Specific examples of nucleotide sequences of genes encoding the amino acid sequence of tripeptide peptidase include:
(6E) the nucleotide sequence as shown in SEQ ID NO: 25;
(6F) a nucleotide sequence derived from the nucleotide sequence as shown in SEQ ID NO: 25 by addition, deletion, or substitution of 1 or a plurality of nucleotides (which is a nucleotide sequence derived from the nucleotide sequence as shown in SEQ ID NO: 25 preferably by substitution, deletion, and/or addition, and more preferably by deletion and/or addition, of 1 or a plurality of nucleotides at either or both of the 5′ terminus and the 3′ terminus) and encoding a polypeptide having tripeptide peptidase activity;
(6G) a nucleotide sequence having 80% or higher, preferably 85% or higher, and more preferably 90% or higher, 95% or higher, 97% or higher, 98% or higher, or 99% or higher sequence identity to the nucleotide sequence as shown in SEQ ID NO: 25 and encoding a polypeptide having tripeptide peptidase activity;
(6H) a partial nucleotide sequence of any of the nucleotide sequences (6E) to (6G) encoding an amino acid sequence of a polypeptide having tripeptide peptidase activity;
(6I) a nucleotide sequence derived from any of the nucleotide sequences (6E) to (6H) by introduction of silent mutation (nucleotide substitution that does not alter amino acids to encode);
(6J) a nucleotide sequence encoding the amino acid sequence of any of the polypeptides (6A) to (6D); and
(6K) a nucleotide sequence comprising, as an exon sequence, any of the nucleotide sequences (6E) to (6J) and one or more intron sequences therein.

In (6F) above, the term “a plurality of” refers to, for example, 2 to 60, 2 to 45, 2 to 30, 2 to 21, 2 to 15, 2 to 6, or 2 or 3.

In (6G) above, “sequence identity” is a value determined by aligning 2 nucleotide sequences, introducing gaps, according to need, so as to maximize the extent of nucleotide consistency therebetween, and determining a percentage (%) of identical nucleotides based on the total number of nucleotides in the nucleotide sequence as shown in SEQ ID NO: 25.

### 7. Protein Involved in Glutathione Import

A protein that relates to glutathione import has functions of incorporating glutathione that is present outside a cell into the cell.

Examples of proteins involved in glutathione import in microorganisms may be one or more proteins selected from among YliA (the glutathione transport system ATP-binding protein), YliB (the glutathione transport system substrate-binding protein), YliC (the glutathione transport system permease protein), and YliD (the glutathione transport system permease protein). The genes encoding the amino acid sequences of YliA, YliB, YliC, and YliD are yliA, yliB, yliC, and yliD, respectively. yliA, yliB, yliC, and yliD form an operon in the genomic DNA of microorganisms and expression thereof is controlled by a promoter located upstream of yliA. The YliA, YliB, YliC, and YliD proteins may be collectively referred to as “YliABCD,” and the yliA, yliB, yliC, and yliD genes may be collectively referred to as “yliABCD.”

The term “a gene encoding a protein involved in glutathione import” refers to a nucleic acid (DNA or RNA, with DNA being preferable) encoding the amino acid sequence of a protein involved in glutathione import, and such gene is included in the genomic DNA in the chromosome of the wild-type microbial strain before disruption of the gene therein.

The microbial strain according to one or more embodiments of the present invention described below preferably has disruptions of one or more genes selected from among yliA, yliB, yliC, and yliD, and more preferably has disruptions of all of yliA, yliB, yliC, and yliD.

Specific examples of the YliA protein (the glutathione transport system ATP-binding protein) include:
(7-1A) a polypeptide consisting of the amino acid sequence as shown in SEQ ID NO: 30;
(7-1B) a polypeptide consisting of an amino acid sequence derived from the amino acid sequence as shown in SEQ ID NO: 30 by addition, deletion, or substitution of 1 or a plurality of amino acids (which is a polypeptide consisting of an amino acid sequence derived from the amino acid sequence as shown in SEQ ID NO: 30 preferably by substitution, deletion, and/or addition, and more preferably by deletion and/or addition, of 1 or a plurality of amino acids at either or both of the N terminus and the C terminus) and having activity as YliA;
(7-1C) a polypeptide consisting of an amino acid sequence having 80% or higher, preferably 85% or higher, and more preferably 90% or higher, 95% or higher, 97% or higher, 98% or higher, or 99% or higher sequence identity to the amino acid sequence as shown in SEQ ID NO: 30 and having activity as YliA; and
(7-1D) a fragment of any of the polypeptides (7-1A) to (7-1C) having activity as YliA.

In (7-1B) to (7-1D) above and (7-1F) to (7-1H) below, a polypeptide “having activity as YliA” has a function of the polypeptide consisting of the amino acid sequence as shown in SEQ ID NO: 30, in particular, ATP-binding activity of the glutathione transport system.

The fragment (7-1D) can be a polypeptide comprising preferably 400 or more, more preferably 500 or more, and more preferably 600 or more amino acids.

In (7-1B) above, the term “a plurality of” refers to, for example, 2 to 20, 2 to 15, 2 to 10, 2 to 7, 2 to 5, 2 to 4, or 2 or 3. Amino acid substitution is preferably conservative amino acid substitution.

In (7-1C) above, “sequence identity” is a value determined by aligning 2 amino acid sequences, introducing gaps, according to need, so as to maximize the extent of amino acid consistency therebetween, and determining a percentage (%) of identical amino acids based on the total number of amino acids in the amino acid sequence as shown in SEQ ID NO: 30.

The term “yliA gene” refers to a nucleic acid (DNA or RNA, with DNA being preferable) encoding the amino acid sequence of yliA and such gene is included in the genomic DNA in the chromosome of the wild-type microbial strain before disruption of the gene therein.

SEQ ID NO: 29 shows an example of DNA encoding the amino acid sequence of E. coli-derived YliA as shown in SEQ ID NO: 30. It should be noted that the nucleotide sequence as shown in SEQ ID NO: 29 is not always present in that state in the genomic DNA of the wild-type microbial strain before disruption of the gene therein. The nucleotide sequence as shown in SEQ ID NO: 29 may be an exon sequence comprising one or more intron sequences therein.

Specific examples of nucleotide sequences of genes encoding the amino acid sequence of YliA or the yliA gene include:
(7-1E) the nucleotide sequence as shown in SEQ ID NO: 29;
(7-1F) a nucleotide sequence derived from the nucleotide sequence as shown in SEQ ID NO: 29 by addition, deletion, or substitution of 1 or a plurality of nucleotides (which is a nucleotide sequence derived from the nucleotide sequence as shown in SEQ ID NO: 29 preferably by substitution, deletion, and/or addition, and more preferably by deletion and/or addition, of 1 or a plurality of nucleotides at either or both of the 5′ terminus and the 3′ terminus) and encoding a polypeptide having activity as YliA;
(7-1G) a nucleotide sequence having 80% or higher, preferably 85% or higher, and more preferably 90% or higher, 95% or higher, 97% or higher, 98% or higher, or 99% or higher sequence identity to the nucleotide sequence as shown in SEQ ID NO: 29 and encoding a polypeptide having activity as YliA;
(7-1H) a partial nucleotide sequence of any of the nucleotide sequences (7-1E) to (7-1G) encoding an amino acid sequence of a polypeptide having activity as YliA;
(7-1I) a nucleotide sequence derived from any of the nucleotide sequences (7-1E) to (7-1H) by introduction of silent mutation (nucleotide substitution that does not alter amino acids to encode);
(7-1J) a nucleotide sequence encoding the amino acid sequence of any of the polypeptides (7-1A) to (7-1D); and
(7-1K) a nucleotide sequence comprising, as an exon sequence, any of the nucleotide sequences (7-1E) to (7-1J) and one or more intron sequences therein.

In (7-1F) above, the term “a plurality of” refers to, for example, 2 to 60, 2 to 45, 2 to 30, 2 to 21, 2 to 15, 2 to 6, or 2 or 3.

In (7-1G) above, “sequence identity” is a value determined by aligning 2 nucleotide sequences, introducing gaps, according to need, so as to maximize the extent of nucleotide consistency therebetween, and determining a percentage (%) of identical nucleotides based on the total number of nucleotides in the nucleotide sequence as shown in SEQ ID NO: 29.

Specific examples of the YliB protein (the glutathione transport system substrate-binding protein) include:
(7-2A) a polypeptide consisting of the amino acid sequence as shown in SEQ ID NO: 32;
(7-2B) a polypeptide consisting of an amino acid sequence derived from the amino acid sequence as shown in SEQ ID NO: 32 by addition, deletion, or substitution of 1 or a plurality of amino acids (which is a polypeptide consisting of an amino acid sequence derived from the amino acid sequence as shown in SEQ ID NO: 32 preferably by substitution, deletion, and/or addition, and more preferably by deletion and/or addition, of 1 or a plurality of amino acids at either or both of the N terminus and the C terminus) and having activity as YliB;
(7-2C) a polypeptide consisting of an amino acid sequence having 80% or higher, preferably 85% or higher, and more preferably 90% or higher, 95% or higher, 97% or higher, 98% or higher, or 99% or higher sequence identity to the amino acid sequence as shown in SEQ ID NO: 32 and having activity as YliB; and
(7-2D) a fragment of any of the polypeptides (7-2A) to (7-2C) having activity as YliB.

In (7-2B) to (7-2D) above and (7-2F) to (7-2H) below, a polypeptide “having activity as YliB” has functions of the polypeptide consisting of the amino acid sequence as shown in SEQ ID NO: 32, in particular, substrate-binding activity of the glutathione transport system.

The fragment (7-2D) can be a polypeptide comprising preferably 300 or more, more preferably 400 or more, and more preferably 500 or more amino acids.

In (7-2B) above, the term “a plurality of” refers to, for example, 2 to 20, 2 to 15, 2 to 10, 2 to 7, 2 to 5, 2 to 4, or 2 or 3. Amino acid substitution is preferably conservative amino acid substitution.

In (7-2C) above, “sequence identity” is a value determined by aligning 2 amino acid sequences, introducing gaps, according to need, so as to maximize the extent of amino acid consistency therebetween, and determining a percentage (%) of identical amino acids based on the total number of amino acids in the amino acid sequence as shown in SEQ ID NO: 32.

The term “yliB gene” refers to a nucleic acid (DNA or RNA, with DNA being preferable) encoding the amino acid sequence of yliB and such gene is included in the genomic DNA in the chromosome of the wild-type microbial strain before disruption of the gene therein.

SEQ ID NO: 31 shows an example of DNA encoding the amino acid sequence of E. coli-derived YliB as shown in SEQ ID NO: 32. It should be noted that the nucleotide sequence as shown in SEQ ID NO: 31 is not always present in that state in the genomic DNA of the wild-type microbial strain before disruption of the gene therein. The nucleotide sequence as shown in SEQ ID NO: 31 may be an exon sequence comprising one or more intron sequences therein.

Specific examples of nucleotide sequences of genes encoding the amino acid sequence of YliB or the yliB gene include:
(7-2E) the nucleotide sequence as shown in SEQ ID NO: 31;
(7-2F) a nucleotide sequence derived from the nucleotide sequence as shown in SEQ ID NO: 31 by addition, deletion, or substitution of 1 or a plurality of nucleotides (which is a nucleotide sequence derived from the nucleotide sequence as shown in SEQ ID NO: 31 preferably by substitution, deletion, and/or addition, and more preferably by deletion and/or addition, of 1 or a plurality of nucleotides at either or both of the 5′ terminus and the 3′ terminus) and encoding a polypeptide having activity as YliB;
(7-2G) a nucleotide sequence having 80% or higher, preferably 85% or higher, and more preferably 90% or higher, 95% or higher, 97% or higher, 98% or higher, or 99% or higher sequence identity to the nucleotide sequence as shown in SEQ ID NO: 31 and encoding a polypeptide having activity as YliB;
(7-2H) a partial nucleotide sequence of any of the nucleotide sequences (7-2E) to (7-2G) encoding an amino acid sequence of a polypeptide having activity as YliB;
(7-2I) a nucleotide sequence derived from any of the nucleotide sequences (7-2E) to (7-2H) by introduction of silent mutation (nucleotide substitution that does not alter amino acids to encode);
(7-2J) a nucleotide sequence encoding the amino acid sequence of any of the polypeptides (7-2A) to (7-2D); and
(7-2K) a nucleotide sequence comprising, as an exon sequence, any of the nucleotide sequences (7-2E) to (7-2J) and one or more intron sequences therein.

In (7-2F) above, the term “a plurality of” refers to, for example, 2 to 60, 2 to 45, 2 to 30, 2 to 21, 2 to 15, 2 to 6, or 2 or 3.

In (7-2G) above, “sequence identity” is a value determined by aligning 2 nucleotide sequences, introducing gaps, according to need, so as to maximize the extent of nucleotide consistency therebetween, and determining a percentage (%) of identical nucleotides based on the total number of nucleotides in the nucleotide sequence as shown in SEQ ID NO: 31.

Specific examples of the YliC protein (the glutathione transport system permease protein) include:
(7-3A) a polypeptide consisting of the amino acid sequence as shown in SEQ ID NO: 34;
(7-3B) a polypeptide consisting of an amino acid sequence derived from the amino acid sequence as shown in SEQ ID NO: 34 by addition, deletion, or substitution of 1 or a plurality of amino acids (which is a polypeptide consisting of an amino acid sequence derived from the amino acid sequence as shown in SEQ ID NO: 34 preferably by substitution, deletion, and/or addition, and more preferably by deletion and/or addition, of 1 or a plurality of amino acids at either or both of the N terminus and the C terminus) and having activity as YliC;
(7-3C) a polypeptide consisting of an amino acid sequence having 80% or higher, preferably 85% or higher, and more preferably 90% or higher, 95% or higher, 97% or higher, 98% or higher, or 99% or higher sequence identity to the amino acid sequence as shown in SEQ ID NO: 34 and having activity as YliC; and
(7-3D) a fragment of any of the polypeptides (7-3A) to (7-3C) having activity as YliC.

In (7-3B) to (7-3D) above and (7-3F) to (7-3H) below, a polypeptide “having activity as YliC” has functions of the polypeptide consisting of the amino acid sequence as shown in SEQ ID NO: 34, in particular, permease activity of the glutathione transport system.

The fragment (7-3D) can be a polypeptide comprising preferably 200 or more, more preferably 250 or more, and more preferably 300 or more amino acids.

In (7-3B) above, the term “a plurality of” refers to, for example, 2 to 20, 2 to 15, 2 to 10, 2 to 7, 2 to 5, 2 to 4, or 2 or 3. Amino acid substitution is preferably conservative amino acid substitution.

In (7-3C) above, “sequence identity” is a value determined by aligning 2 amino acid sequences, introducing gaps, according to need, so as to maximize the extent of amino acid consistency therebetween, and determining a percentage (%) of identical amino acids based on the total number of amino acids in the amino acid sequence as shown in SEQ ID NO: 34.

The term “yliC gene” refers to a nucleic acid (DNA or RNA, with DNA being preferable) encoding the amino acid sequence of YliC and such gene is included in the genomic DNA in the chromosome of the wild-type microbial strain before disruption of the gene therein.

SEQ ID NO: 33 shows an example of DNA encoding the amino acid sequence of E. coli-derived YliC as shown in SEQ ID NO: 34. It should be noted that the nucleotide sequence as shown in SEQ ID NO: 33 is not always present in that state in the genomic DNA of the wild-type microbial strain before disruption of the gene therein. The nucleotide sequence as shown in SEQ ID NO: 33 may be an exon sequence comprising one or more intron sequences therein.

Specific examples of nucleotide sequences of genes encoding the amino acid sequence YliC or the yliC gene include:
(7-3E) the nucleotide sequence as shown in SEQ ID NO: 33;
(7-3F) a nucleotide sequence derived from the nucleotide sequence as shown in SEQ ID NO: 33 by addition, deletion, or substitution of 1 or a plurality of nucleotides (which is a nucleotide sequence derived from the nucleotide sequence as shown in SEQ ID NO: 33 preferably by substitution, deletion, and/or addition, and more preferably by deletion and/or addition, of 1 or a plurality of nucleotides at either or both of the 5′ terminus and the 3′ terminus) and encoding a polypeptide having activity as YliC;
(7-3G) a nucleotide sequence having 80% or higher, preferably 85% or higher, and more preferably 90% or higher, 95% or higher, 97% or higher, 98% or higher, or 99% or higher sequence identity to the nucleotide sequence as shown in SEQ ID NO: 33 and encoding a polypeptide having activity as YliC;
(7-3H) a partial nucleotide sequence of any of the nucleotide sequences (7-3E) to (7-3G) encoding an amino acid sequence of a polypeptide having activity as YliC;
(7-3I) a nucleotide sequence derived from any of the nucleotide sequences (7-3E) to (7-3H) by introduction of silent mutation (nucleotide substitution that does not alter amino acids to encode);
(7-3J) a nucleotide sequence encoding the amino acid sequence of any of the polypeptides (7-3A) to (7-3D); and
(7-3K) a nucleotide sequence comprising, as an exon sequence, any of the nucleotide sequences (7-3E) to (7-3J) and one or more intron sequences therein.

In (7-3F) above, the term “a plurality of” refers to, for example, 2 to 60, 2 to 45, 2 to 30, 2 to 21, 2 to 15, 2 to 6, or 2 or 3.

In (7-3G) above, “sequence identity” is a value determined by aligning 2 nucleotide sequences, introducing gaps, according to need, so as to maximize the extent of nucleotide consistency therebetween, and determining a percentage (%) of identical nucleotides based on the total number of nucleotides in the nucleotide sequence as shown in SEQ ID NO: 33.

Specific examples of the YliD protein (the glutathione transport system permease protein) include:
(7-4A) a polypeptide consisting of the amino acid sequence as shown in SEQ ID NO: 36;
(7-4B) a polypeptide consisting of an amino acid sequence derived from the amino acid sequence as shown in SEQ ID NO: 36 by addition, deletion, or substitution of 1 or a plurality of amino acids (which is a polypeptide consisting of an amino acid sequence derived from the amino acid sequence as shown in SEQ ID NO: 36 preferably by substitution, deletion, and/or addition, and more preferably by deletion and/or addition, of 1 or a plurality of amino acids at either or both of the N terminus and the C terminus) and having activity as YliD;
(7-4C) a polypeptide consisting of an amino acid sequence having 80% or higher, preferably 85% or higher, and more preferably 90% or higher, 95% or higher, 97% or higher, 98% or higher, or 99% or higher sequence identity to the amino acid sequence as shown in SEQ ID NO: 36 and having activity as YliD; and
(7-4D) a fragment of any of the polypeptides (7-4A) to (7-4C) having activity as YliD.

In (7-4B) to (7-4D) above and (7-4F) to (7-4H) below, a polypeptide “having activity as YliD” has functions of the polypeptide consisting of the amino acid sequence as shown in SEQ ID NO: 36, in particular, permease activity of the glutathione transport system.

The fragment (7-4D) can be a polypeptide comprising preferably 200 or more, more preferably 250 or more, and more preferably 300 or more amino acids.

In (7-4B) above, the term “a plurality of” refers to, for example, 2 to 20, 2 to 15, 2 to 10, 2 to 7, 2 to 5, 2 to 4, or 2 or 3. Amino acid substitution is preferably conservative amino acid substitution.

In (7-4C) above, “sequence identity” is a value determined by aligning 2 amino acid sequences, introducing gaps, according to need, so as to maximize the extent of amino acid consistency therebetween, and determining a percentage (%) of identical amino acids based on the total number of amino acids in the amino acid sequence as shown in SEQ ID NO: 36.

The term “yliD gene” refers to a nucleic acid (DNA or RNA, with DNA being preferable) encoding the amino acid sequence of yliD, and such gene is included in the genomic DNA in the chromosome of the wild-type microbial strain before disruption of the gene therein.

SEQ ID NO: 35 shows an example of DNA encoding the amino acid sequence of E. coli-derived YliD as shown in SEQ ID NO: 36. It should be noted that the nucleotide sequence as shown in SEQ ID NO: 35 is not always present in that state in the genomic DNA of the wild-type microbial strain before disruption of the gene therein. The nucleotide sequence as shown in SEQ ID NO: 35 may be an exon sequence comprising one or more intron sequences therein.

Specific examples of nucleotide sequences of genes encoding the amino acid sequence YliD or the yliD gene include:
(7-4E) the nucleotide sequence as shown in SEQ ID NO: 35;
(7-4F) a nucleotide sequence derived from the nucleotide sequence as shown in SEQ ID NO: 35 by addition, deletion, or substitution of 1 or a plurality of nucleotides (which is a nucleotide sequence derived from the nucleotide sequence as shown in SEQ ID NO: 35 preferably by substitution, deletion, and/or addition, and more preferably by deletion and/or addition, of 1 or a plurality of nucleotides at either or both of the 5′ terminus and the 3′ terminus) and encoding a polypeptide having activity as YliD;
(7-4G) a nucleotide sequence having 80% or higher, preferably 85% or higher, and more preferably 90% or higher, 95% or higher, 97% or higher, 98% or higher, or 99% or higher sequence identity to the nucleotide sequence as shown in SEQ ID NO: 35 and encoding a polypeptide having activity as YliD;
(7-4H) a partial nucleotide sequence of any of the nucleotide sequences (7-4E) to (7-4G) encoding an amino acid sequence of a polypeptide having activity as YliD;
(7-4I) a nucleotide sequence derived from any of the nucleotide sequences (7-4E) to (7-4H) by introduction of silent mutation (nucleotide substitution that does not alter amino acids to encode);
(7-4J) a nucleotide sequence encoding the amino acid sequence of any of the polypeptides (7-4A) to (7-4D); and
(7-4K) a nucleotide sequence comprising, as an exon sequence, any of the nucleotide sequences (7-4E) to (7-4J) and one or more intron sequences therein.

In (7-4F) above, the term “a plurality of” refers to, for example, 2 to 60, 2 to 45, 2 to 30, 2 to 21, 2 to 15, 2 to 6, or 2 or 3.

In (7-4G) above, “sequence identity” is a value determined by aligning 2 nucleotide sequences, introducing gaps, according to need, so as to maximize the extent of nucleotide consistency therebetween, and determining a percentage (%) of identical nucleotides based on the total number of nucleotides in the nucleotide sequence as shown in SEQ ID NO: 35.

### 8. Glutathione Reductase

Glutathione reductase (EC:1.8.1.7) is an enzyme that catalyzes a reaction of reducing oxidized glutathione (glutathione disulfide) in the presence of NADPH to generate reduced glutathione.

Specific examples of glutathione reductases include:
(8A) a polypeptide consisting of the amino acid sequence as shown in SEQ ID NO:28;
(8B) a polypeptide consisting of an amino acid sequence derived from the amino acid sequence as shown in SEQ ID NO:28 by addition, deletion, or substitution of 1 or a plurality of amino acids (which is a polypeptide consisting of an amino acid sequence derived from the amino acid sequence as shown in SEQ ID NO:28 preferably by substitution, deletion, and/or addition, and more preferably by deletion and/or addition, of 1 or a plurality of amino acids at either or both of the N terminus and the C terminus) and having glutathione reductase activity;
(8C) a polypeptide consisting of an amino acid sequence having 80% or higher, preferably 85% or higher, and more preferably 90% or higher, 95% or higher, 97% or higher, 98% or higher, or 99% or higher sequence identity to the amino acid sequence as shown in SEQ ID NO:28 and having glutathione reductase activity; and
(8D) a fragment of any of the polypeptides (8A) to (8C) having glutathione reductase activity.

The fragment (8D) can be a polypeptide comprising preferably 200 or more, more preferably 300 or more, and more preferably 400 or more amino acids.

In (8B) above, the term “a plurality of” refers to, for example, 2 to 20, 2 to 15, 2 to 10, 2 to 7, 2 to 5, 2 to 4, or 2 or 3. Amino acid substitution is preferably conservative amino acid substitution.

In (8C) above, “sequence identity” is a value determined by aligning 2 amino acid sequences, introducing gaps, according to need, so as to maximize the extent of amino acid consistency therebetween, and determining a percentage (%) of identical amino acids based on the total number of amino acids in the amino acid sequence as shown in SEQ ID NO: 28.

The term “a gene encoding glutathione reductase (EC:1.8.1.7)” refers to a nucleic acid (DNA or RNA, with DNA being preferable) encoding the amino acid sequence of glutathione reductase, and such gene is included in the genomic DNA in the chromosome of the wild-type microorganism before disruption of glutathione reductase therein.

SEQ ID NO: 27 shows an example of DNA encoding the amino acid sequence of E. coli-derived glutathione reductase as shown in SEQ ID NO: 28. It should be noted that the nucleotide sequence as shown in SEQ ID NO: 27 is not always present in that state in the genomic DNA of the wild-type microorganism. The nucleotide sequence as shown in SEQ ID NO: 27 may be an exon sequence comprising one or more intron sequences therein.

Specific examples of nucleotide sequences of genes encoding the amino acid sequence of glutathione reductase include:
(8E) the nucleotide sequence as shown in SEQ ID NO:27;
(8F) a nucleotide sequence derived from the nucleotide sequence as shown in SEQ ID NO:27 by addition, deletion, or substitution of 1 or a plurality of nucleotides (which is a nucleotide sequence derived from the nucleotide sequence as shown in SEQ ID NO:27 preferably by substitution, deletion, and/or addition, and more preferably by deletion and/or addition, of 1 or a plurality of nucleotides at either or both of the 5′ terminus and the 3′ terminus) and encoding a polypeptide having glutathione reductase activity;
(8G) a nucleotide sequence having 80% or higher, preferably 85% or higher, and more preferably 90% or higher, 95% or higher, 97% or higher, 98% or higher, or 99% or higher sequence identity to the nucleotide sequence as shown in SEQ ID NO:27 and encoding a polypeptide having glutathione reductase activity;
(8H) a partial nucleotide sequence of any of the nucleotide sequences (8E) to (8G) encoding an amino acid sequence of a polypeptide having glutathione reductase activity;
(8I) a nucleotide sequence derived from any of the nucleotide sequences (8E) to (8H) by introduction of silent mutation (nucleotide substitution that does not alter amino acids to encode);
(8J) a nucleotide sequence encoding the amino acid sequence of any of the polypeptides (8A) to (8D); and
(8K) a nucleotide sequence comprising, as an exon sequence, any of the nucleotide sequences (8E) to (8J) and one or more intron sequences therein.

In (8F) above, the term “a plurality of” refers to, for example, 2 to 60, 2 to 45, 2 to 30, 2 to 21, 2 to 15, 2 to 6, or 2 or 3.

In (8G) above, “sequence identity” is a value determined by aligning 2 nucleotide sequences, introducing gaps, according to need, so as to maximize the extent of nucleotide consistency therebetween, and determining a percentage (%) of identical nucleotides based on the total number of nucleotides in the nucleotide sequence as shown in SEQ ID NO: 27.

### 9. A Protein Involved in Putrescine Export

Putrescine is a compound having the structure shown below and is biosynthesized in microbial cells.

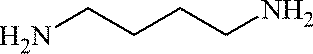

Putrescine is known to have activity of promoting protein synthesis and cell growth in microbial cells. However, the correlation between the putrescine concentration in microbial cells and producibility of γ-glutamylcysteine, bis-γ-glutamylcystine, γ-glutamylcystine, reduced glutathione, and/or oxidized glutathione has not yet been examined.

The present inventors discovered unexpected advantageous effects such that, a microbial strain in which the expression levels of one or more genes encoding proteins involved in putrescine export has been enhanced its yield productivity of γ-glutamylcysteine, bis-γ-glutamylcystine, γ-glutamylcystine, reduced glutathione, and/or oxidized glutathione that is significantly higher than that attained in the host strain. This gene modification is deduced to lower the putrescine concentration in a cell.

A protein involved in putrescine export has functions of discharging putrescine existing in a cell to the outside of the cell.

Examples of proteins involved in putrescine export in microorganisms may be one or more proteins selected from among a substrate-binding protein of the cationic peptide transport system, a permease protein of the cationic peptide transport system, and an ATP-binding protein of the cationic peptide transport system. By enhancing the expression levels of one or more genes encoding any proteins involved in putrescine export, productivity of γ-glutamylcysteine, bis-γ-glutamylcystine, γ-glutamylcystine, reduced glutathione, and/or oxidized glutathione by microbial strains can be enhanced.

An example of a substrate-binding protein of the cationic peptide transport system is SapA. SapA is an E. coli-derived protein. A substrate-binding protein of the cationic peptide transport system is not limited to a protein comprising the amino acid sequence or a conformation similar to that of SapA, as long as it has substrate-binding activity of the cationic peptide transport system and involved in putrescine export.

Examples of permease proteins of the cationic peptide transport system include SapB and SapC. SapB and SapC are E. coli-derived proteins. A permease protein of the cationic peptide transport system is not limited to a protein comprising the amino acid sequence or a conformation similar to that of SapB or SapC, as long as it has permease activity of the cationic peptide transport system and involved in putrescine export.

Examples of ATP-binding proteins of the cationic peptide transport system include SapD and SapF. SapD and SapF are E. coli-derived proteins. An ATP-binding protein of the cationic peptide transport system is not limited to a protein comprising the amino acid sequence or a conformation similar to that of SapD or SapF, as long as it has ATP-binding activity of the cationic peptide transport system and involved in putrescine export.

A protein involved in putrescine export in a microorganism is preferably at least one protein selected from among SapA, SapB, SapC, SapD, and SapF. The genes encoding the amino acid sequences of SapA, SapB, SapC, SapD, and SapF are sapA, sapB, sapC, sapD, and sapF, respectively. sapA, sapB, sapC, sapD, and sapF form an operon in the genomic DNA of the microorganism and expression thereof is controlled by a promoter located upstream of sapA. The SapA, SapB, SapC, SapD, and SapF proteins may be collectively referred to as “SapABCDF,” and the sapA, sapB, sapC, sapD, and sapF genes may be collectively referred to as “sapABCDF.”

The term “a gene encoding a protein involved in putrescine export” refers to a nucleic acid (DNA or RNA, with DNA being preferable) encoding the amino acid sequence of the protein involved in putrescine export, and such gene is included in the genomic DNA in the chromosome of the microbial strain.

In the microbial strain according to one or more embodiments of the present invention described below, it is preferable that the expression levels of one or more genes selected from among sapA, sapB, sapC, sapD, and sapF be enhanced, and it is more preferable that the expression levels of all of the sapA, sapB, sapC, sapD, and sapF genes be enhanced.

Specific examples of the SapA protein (a substrate-binding protein of the cationic peptide transport system) include:
(9-1A) a polypeptide consisting of the amino acid sequence as shown in SEQ ID NO: 40;
(9-1B) a polypeptide consisting of an amino acid sequence derived from the amino acid sequence as shown in SEQ ID NO: 40 by addition, deletion, or substitution of 1 or a plurality of amino acids (which is a polypeptide consisting of an amino acid sequence derived from the amino acid sequence as shown in SEQ ID NO: 40 preferably by substitution, deletion, and/or addition, and more preferably by deletion and/or addition, of 1 or a plurality of amino acids at either or both of the N terminus and the C terminus) and having activity as SapA;
(9-1C) a polypeptide consisting of an amino acid sequence having 80% or higher, preferably 85% or higher, and more preferably 90% or higher, 95% or higher, 97% or higher, 98% or higher, or 99% or higher sequence identity to the amino acid sequence as shown in SEQ ID NO: 40 and having activity as SapA; and
(9-1D) a fragment of any of the polypeptides (9-1A) to (9-1C) having activity as SapA.

In (9-1B) to (9-1D) above and (9-1F) to (9-1H) below, a polypeptide “having activity as SapA” has functions of the polypeptide consisting of the amino acid sequence as shown in SEQ ID NO: 40, in particular, substrate-binding activity of the cationic peptide transport system.

The fragment (9-1D) can be a polypeptide comprising preferably 200 or more, more preferably 300 or more, more preferably 400 or more, and more preferably 500 or more amino acids.

In (9-1B) above, the term “a plurality of” refers to, for example, 2 to 20, 2 to 15, 2 to 10, 2 to 7, 2 to 5, 2 to 4, or 2 or 3. Amino acid substitution is preferably conservative amino acid substitution.

In (9-1C) above, “sequence identity” is a value determined by aligning 2 amino acid sequences, introducing gaps, according to need, so as to maximize the extent of amino acid consistency therebetween, and determining a percentage (%) of identical amino acids based on the total number of amino acids in the amino acid sequence as shown in SEQ ID NO: 40.

The term “sapA gene” refers to a nucleic acid (DNA or RNA, with DNA being preferable) encoding the amino acid sequence of SapA, and such gene is included in the genomic DNA in the chromosome of the microbial strain.

SEQ ID NO: 39 shows an example of DNA encoding the amino acid sequence of E. coli-derived SapA as shown in SEQ ID NO: 40. It should be noted that the nucleotide sequence as shown in SEQ ID NO: 39 is not always present in that state in the genomic DNA of the wild-type microbial strain. The nucleotide sequence as shown in SEQ ID NO: 39 may be an exon sequence comprising one or more intron sequences therein.

Specific examples of nucleotide sequences of genes encoding the amino acid sequence of SapA or the sapA gene include:
(9-1E) the nucleotide sequence as shown in SEQ ID NO: 39;
(9-1F) a nucleotide sequence derived from the nucleotide sequence as shown in SEQ ID NO: 3by addition, deletion, or substitution of 1 or a plurality of nucleotides (which is a nucleotide sequence derived from the nucleotide sequence as shown in SEQ ID NO: 3preferably by substitution, deletion, and/or addition, and more preferably by deletion and/or addition, of 1 or a plurality of nucleotides at either or both of the 5′ terminus and the 3′ terminus) and encoding a polypeptide having activity as SapA;
(9-1G) a nucleotide sequence having 80% or higher, preferably 85% or higher, and more preferably 90% or higher, 95% or higher, 97% or higher, 98% or higher, or 99% or higher sequence identity to the nucleotide sequence as shown in SEQ ID NO: 3and encoding a polypeptide having activity as SapA;
(9-1H) a partial nucleotide sequence of any of the nucleotide sequences (9-1E) to (9-1G) encoding an amino acid sequence of a polypeptide having activity as SapA;
(9-1I) a nucleotide sequence derived from any of the nucleotide sequences (9-1E) to (9-1H) by introduction of silent mutation (nucleotide substitution that does not alter amino acids to encode);
(9-1J) a nucleotide sequence encoding the amino acid sequence of any of the polypeptides (9-1A) to (9-1D); and
(9-1K) a nucleotide sequence comprising, as an exon sequence, any of the nucleotide sequences (9-1E) to (9-1J) and one or more intron sequences therein.

In (9-1F) above, the term “a plurality of” refers to, for example, 2 to 60, 2 to 45, 2 to 30, 2 to 21, 2 to 15, 2 to 6, or 2 or 3.

In (9-1G) above, “sequence identity” is a value determined by aligning 2 nucleotide sequences, introducing gaps, according to need, so as to maximize the extent of nucleotide consistency therebetween, and determining a percentage (%) of identical nucleotides based on the total number of nucleotides in the nucleotide sequence as shown in SEQ ID NO: 39.

Specific examples of the SapB protein (a permease protein of the cationic peptide transport system) include:
(9-2A) a polypeptide consisting of the amino acid sequence as shown in SEQ ID NO: 42;
(9-2B) a polypeptide consisting of an amino acid sequence derived from the amino acid sequence as shown in SEQ ID NO: 42 by addition, deletion, or substitution of 1 or a plurality of amino acids (which is a polypeptide consisting of an amino acid sequence derived from the amino acid sequence as shown in SEQ ID NO: 42 preferably by substitution, deletion, and/or addition, and more preferably by deletion and/or addition, of 1 or a plurality of amino acids at either or both of the N terminus and the C terminus) and having activity as SapB;
(9-2C) a polypeptide consisting of an amino acid sequence having 80% or higher, preferably 85% or higher, and more preferably 90% or higher, 95% or higher, 97% or higher, 98% or higher, or 99% or higher sequence identity to the amino acid sequence as shown in SEQ ID NO: 42 and having activity as SapB; and
(9-2D) a fragment of any of the polypeptides (9-2A) to (9-2C) having activity as SapB.

In (9-2B) to (9-2D) above and (9-2F) to (9-2H) below, a polypeptide “having activity as SapB” has functions of the polypeptide consisting of the amino acid sequence as shown in SEQ ID NO: 42, in particular, permease activity of the cationic peptide transport system.

The fragment (9-2D) can be a polypeptide comprising preferably 200 or more, more preferably 250 or more, and more preferably 300 or more amino acids.

In (9-2B) above, the term “a plurality of” refers to, for example, 2 to 20, 2 to 15, 2 to 10, 2 to 7, 2 to 5, 2 to 4, or 2 or 3. Amino acid substitution is preferably conservative amino acid substitution.

In (9-2C) above, “sequence identity” is a value determined by aligning 2 amino acid sequences, introducing gaps, according to need, so as to maximize the extent of amino acid consistency therebetween, and determining a percentage (%) of identical amino acids based on the total number of amino acids in the amino acid sequence as shown in SEQ ID NO: 42.

The term “sapB gene” refers to a nucleic acid (DNA or RNA, with DNA being preferable) encoding the amino acid sequence of SapB, and such gene is included in the genomic DNA in the chromosome of the microbial strain.

SEQ ID NO: 41 shows an example of DNA encoding the amino acid sequence of E. coli-derived SapB as shown in SEQ ID NO: 42. It should be noted that the nucleotide sequence as shown in SEQ ID NO: 41 is not always present in that state in the genomic DNA of the microbial strain. The nucleotide sequence as shown in SEQ ID NO: 41 may be an exon sequence comprising one or more intron sequences therein.

Specific examples of nucleotide sequences of genes encoding the amino acid sequence of SapB or the sapB gene include:
(9-2E) the nucleotide sequence as shown in SEQ ID NO: 41;
(9-2F) a nucleotide sequence derived from the nucleotide sequence as shown in SEQ ID NO: 41 by addition, deletion, or substitution of 1 or a plurality of nucleotides (which is a nucleotide sequence derived from the nucleotide sequence as shown in SEQ ID NO: 41 preferably by substitution, deletion, and/or addition, and more preferably by deletion and/or addition, of 1 or a plurality of nucleotides at either or both of the 5′ terminus and the 3′ terminus) and encoding a polypeptide having activity as SapB;
(9-2G) a nucleotide sequence having 80% or higher, preferably 85% or higher, and more preferably 90% or higher, 95% or higher, 97% or higher, 98% or higher, or 99% or higher sequence identity to the nucleotide sequence as shown in SEQ ID NO: 41 and encoding a polypeptide having activity as SapB;
(9-2H) a partial nucleotide sequence of any of the nucleotide sequences (9-2E) to (9-2G) encoding an amino acid sequence of a polypeptide having activity as SapB;
(9-2I) a nucleotide sequence derived from any of the nucleotide sequences (9-2E) to (9-2H) by introduction of silent mutation (nucleotide substitution that does not alter amino acids to encode);
(9-2J) a nucleotide sequence encoding the amino acid sequence of any of the polypeptides (9-2A) to (9-2D); and
(9-2K) a nucleotide sequence comprising, as an exon sequence, any of the nucleotide sequences (9-2E) to (9-2J) and one or more intron sequences therein.

In (9-2F) above, the term “a plurality of” refers to, for example, 2 to 60, 2 to 45, 2 to 30, 2 to 21, 2 to 15, 2 to 6, or 2 or 3.

In (9-2G) above, “sequence identity” is a value determined by aligning 2 nucleotide sequences, introducing gaps, according to need, so as to maximize the extent of nucleotide consistency therebetween, and determining a percentage (%) of identical nucleotides based on the total number of nucleotides in the nucleotide sequence as shown in SEQ ID NO: 41.

Specific examples of the SapC protein (a permease protein of the cationic peptide transport system) include:
(9-3A) a polypeptide consisting of the amino acid sequence as shown in SEQ ID NO: 44;
(9-3B) a polypeptide consisting of an amino acid sequence derived from the amino acid sequence as shown in SEQ ID NO: 44 by addition, deletion, or substitution of 1 or a plurality of amino acids (which is a polypeptide consisting of an amino acid sequence derived from the amino acid sequence as shown in SEQ ID NO: 44 preferably by substitution, deletion, and/or addition, and more preferably by deletion and/or addition, of 1 or a plurality of amino acids at either or both of the N terminus and the C terminus) and having activity as SapC;
(9-3C) a polypeptide consisting of an amino acid sequence having 80% or higher, preferably 85% or higher, and more preferably 90% or higher, 95% or higher, 97% or higher, 98% or higher, or 99% or higher sequence identity to the amino acid sequence as shown in SEQ ID NO: 44 and having activity as SapC; and
(9-3D) a fragment of any of the polypeptides (9-3A) to (9-3C) having activity as SapC.

In (9-3B) to (9-3D) above and (9-3F) to (9-3H) below, a polypeptide “having activity as SapC” has functions of the polypeptide consisting of the amino acid sequence as shown in SEQ ID NO: 44, in particular, permease activity of the cationic peptide transport system.

The fragment (9-3D) can be a polypeptide comprising preferably 200 or more, and more preferably 250 or more amino acids.

In (9-3B) above, the term “a plurality of” refers to, for example, 2 to 20, 2 to 15, 2 to 10, 2 to 7, 2 to 5, 2 to 4, or 2 or 3. Amino acid substitution is preferably conservative amino acid substitution.

In (9-3C) above, “sequence identity” is a value determined by aligning 2 amino acid sequences, introducing gaps, according to need, so as to maximize the extent of amino acid consistency therebetween, and determining a percentage (%) of identical amino acids based on the total number of amino acids in the amino acid sequence as shown in SEQ ID NO: 44.

The term “sapC gene” refers to a nucleic acid (DNA or RNA, with DNA being preferable) encoding the amino acid sequence of SapC, and such gene is included in the genomic DNA in the chromosome of the microbial strain.

SEQ ID NO: 43 shows an example of DNA encoding the amino acid sequence of E. coli-derived SapC as shown in SEQ ID NO: 44. It should be noted that the nucleotide sequence as shown in SEQ ID NO: 43 is not always present in that state in the genomic DNA of the microbial strain. The nucleotide sequence as shown in SEQ ID NO: 43 may be an exon sequence comprising one or more intron sequences therein.

Specific examples of nucleotide sequences of genes encoding the amino acid sequence of SapC or the sapC gene include:
(9-3E) the nucleotide sequence as shown in SEQ ID NO: 43;
(9-3F) a nucleotide sequence derived from the nucleotide sequence as shown in SEQ ID NO: 43 by addition, deletion, or substitution of 1 or a plurality of nucleotides (which is a nucleotide sequence derived from the nucleotide sequence as shown in SEQ ID NO: 43 preferably by substitution, deletion, and/or addition, and more preferably by deletion and/or addition, of 1 or a plurality of nucleotides at either or both of the 5′ terminus and the 3′ terminus) and encoding a polypeptide having activity as SapC;
(9-3G) a nucleotide sequence having 80% or higher, preferably 85% or higher, and more preferably 90% or higher, 95% or higher, 97% or higher, 98% or higher, or 99% or higher sequence identity to the nucleotide sequence as shown in SEQ ID NO: 43 and encoding a polypeptide having activity as SapC;
(9-3H) a partial nucleotide sequence of any of the nucleotide sequences (9-3E) to (9-3G) encoding an amino acid sequence of a polypeptide having activity as SapC;
(9-3I) a nucleotide sequence derived from any of the nucleotide sequences (9-3E) to (9-3H) by introduction of silent mutation (nucleotide substitution that does not alter amino acids to encode);
(9-3J) a nucleotide sequence encoding the amino acid sequence of any of the polypeptides (9-3A) to (9-3D); and
(9-3K) a nucleotide sequence comprising, as an exon sequence, any of the nucleotide sequences (9-3E) to (9-3J) and one or more intron sequences therein.

In (9-3F) above, the term “a plurality of” refers to, for example, 2 to 60, 2 to 45, 2 to 30, 2 to 21, 2 to 15, 2 to 6, or 2 or 3.

In (9-3G) above, “sequence identity” is a value determined by aligning 2 nucleotide sequences, introducing gaps, according to need, so as to maximize the extent of nucleotide consistency therebetween, and determining a percentage (%) of identical nucleotides based on the total number of nucleotides in the nucleotide sequence as shown in SEQ ID NO: 43.

Specific examples of the SapD protein (an ATP-binding protein of the cationic peptide transport system) include:
(9-4A) a polypeptide consisting of the amino acid sequence as shown in SEQ ID NO: 46;
(9-4B) a polypeptide consisting of an amino acid sequence derived from the amino acid sequence as shown in SEQ ID NO: 46 by addition, deletion, or substitution of 1 or a plurality of amino acids (which is a polypeptide consisting of an amino acid sequence derived from the amino acid sequence as shown in SEQ ID NO: 46 preferably by substitution, deletion, and/or addition, and more preferably by deletion and/or addition, of 1 or a plurality of amino acids at either or both of the N terminus and the C terminus) and having activity as SapD;
(9-4C) a polypeptide consisting of an amino acid sequence having 80% or higher, preferably 85% or higher, and more preferably 90% or higher, 95% or higher, 97% or higher, 98% or higher, or 99% or higher sequence identity to the amino acid sequence as shown in SEQ ID NO: 46 and having activity as SapD; and
(9-4D) a fragment of any of the polypeptides (9-4A) to (9-4C) having activity as SapD.

In (9-4B) to (9-4D) above and (9-4F) to (9-4H) below, a polypeptide “having activity as SapD” has functions of the polypeptide consisting of the amino acid sequence as shown in SEQ ID NO:46, in particular, ATP-binding activity of the cationic peptide transport system.

The fragment (9-4D) can be a polypeptide comprising preferably 200 or more, more preferably 250 or more, and more preferably 300 or more amino acids.

In (9-4B) above, the term “a plurality of” refers to, for example, 2 to 20, 2 to 15, 2 to 10, 2 to 7, 2 to 5, 2 to 4, or 2 or 3. Amino acid substitution is preferably conservative amino acid substitution.

In (9-4C) above, “sequence identity” is a value determined by aligning 2 amino acid sequences, introducing gaps, according to need, so as to maximize the extent of amino acid consistency therebetween, and determining a percentage (%) of identical amino acids based on the total number of amino acids in the amino acid sequence as shown in SEQ ID NO: 46.

The term “sapD gene” refers to a nucleic acid (DNA or RNA, with DNA being preferable) encoding the amino acid sequence of SapD, and such gene is included in the genomic DNA in the chromosome of the microbial strain.

SEQ ID NO: 45 shows an example of DNA encoding the amino acid sequence of E. coli-derived SapD as shown in SEQ ID NO: 46. It should be noted that the nucleotide sequence as shown in SEQ ID NO: 45 is not always present in that state in the genomic DNA of the microbial strain. The nucleotide sequence as shown in SEQ ID NO: 45 may be an exon sequence comprising one or more intron sequences therein.

Specific examples of nucleotide sequences of genes encoding the amino acid sequence of SapD or the sapD gene include:
(9-4E) the nucleotide sequence as shown in SEQ ID NO: 45;
(9-4F) a nucleotide sequence derived from the nucleotide sequence as shown in SEQ ID NO: 45 by addition, deletion, or substitution of 1 or a plurality of nucleotides (which is a nucleotide sequence derived from the nucleotide sequence as shown in SEQ ID NO: 45 preferably by substitution, deletion, and/or addition, and more preferably by deletion and/or addition, of 1 or a plurality of nucleotides at either or both of the 5′ terminus and the 3′ terminus) and encoding a polypeptide having activity as SapD;
(9-4G) a nucleotide sequence having 80% or higher, preferably 85% or higher, and more preferably 90% or higher, 95% or higher, 97% or higher, 98% or higher, or 99% or higher sequence identity to the nucleotide sequence as shown in SEQ ID NO: 45 and encoding a polypeptide having activity as SapD;
(9-4H) a partial nucleotide sequence of any of the nucleotide sequences (9-4E) to (9-4G) encoding an amino acid sequence of a polypeptide having activity as SapD;
(9-4I) a nucleotide sequence derived from any of the nucleotide sequences (9-4E) to (9-4H) by introduction of silent mutation (nucleotide substitution that does not alter amino acids to encode);
(9-4J) a nucleotide sequence encoding the amino acid sequence of any of the polypeptides (9-4A) to (9-4D); and
(9-4K) a nucleotide sequence comprising, as an exon sequence, any of the nucleotide sequences (9-4E) to (9-4J) and one or more intron sequences therein.

In (9-4F) above, the term “a plurality of” refers to, for example, 2 to 60, 2 to 45, 2 to 30, 2 to 21, 2 to 15, 2 to 6, or 2 or 3.

In (9-4G) above, “sequence identity” is a value determined by aligning 2 nucleotide sequences, introducing gaps, according to need, so as to maximize the extent of nucleotide consistency therebetween, and determining a percentage (%) of identical nucleotides based on the total number of nucleotides in the nucleotide sequence as shown in SEQ ID NO: 45.

Specific examples of the SapF protein (an ATP-binding protein of the cationic peptide transport system) include:
(9-5A) a polypeptide consisting of the amino acid sequence as shown in SEQ ID NO: 48;
(9-5B) a polypeptide consisting of an amino acid sequence derived from the amino acid sequence as shown in SEQ ID NO: 48 by addition, deletion, or substitution of 1 or a plurality of amino acids (which is a polypeptide consisting of an amino acid sequence derived from the amino acid sequence as shown in SEQ ID NO: 48 preferably by substitution, deletion, and/or addition, and more preferably by deletion and/or addition, of 1 or a plurality of amino acids at either or both of the N terminus and the C terminus) and having activity as SapF;
(9-5C) a polypeptide consisting of an amino acid sequence having 80% or higher, preferably 85% or higher, and more preferably 90% or higher, 95% or higher, 97% or higher, 98% or higher, or 99% or higher sequence identity to the amino acid sequence as shown in SEQ ID NO: 48 and having activity as SapF; and
(9-5D) a fragment of any of the polypeptides (9-5A) to (9-5C) having activity as SapF.

In (9-5B) to (9-5D) above and (9-5F) to (9-5H) below, a polypeptide “having activity as SapF” has functions of the polypeptide consisting of the amino acid sequence as shown in SEQ ID NO: 48, in particular, ATP-binding activity of the cationic peptide transport system.

The fragment (9-5D) can be a polypeptide comprising preferably 200 or more, and more preferably 250 or more amino acids.

In (9-5B) above, the term “a plurality of” refers to, for example, 2 to 20, 2 to 15, 2 to 10, 2 to 7, 2 to 5, 2 to 4, or 2 or 3. Amino acid substitution is preferably conservative amino acid substitution.

In (9-5C) above, “sequence identity” is a value determined by aligning 2 amino acid sequences, introducing gaps, according to need, so as to maximize the extent of amino acid consistency therebetween, and determining a percentage (%) of identical amino acids based on the total number of amino acids in the amino acid sequence as shown in SEQ ID NO: 48.

The term “sapF gene” refers to a nucleic acid (DNA or RNA, with DNA being preferable) encoding the amino acid sequence of SapF, and such gene is included in the genomic DNA in the chromosome of the microbial strain.

SEQ ID NO: 47 shows an example of DNA encoding the amino acid sequence of E. coli-derived SapF as shown in SEQ ID NO: 48. It should be noted that the nucleotide sequence as shown in SEQ ID NO: 47 is not always present in that state in the genomic DNA of the microbial strain. The nucleotide sequence as shown in SEQ ID NO: 47 may be an exon sequence comprising one or more intron sequences therein.

Specific examples of nucleotide sequences of genes encoding the amino acid sequence of SapF or the sapF gene include:
(9-5E) the nucleotide sequence as shown in SEQ ID NO: 47;
(9-5F) a nucleotide sequence derived from the nucleotide sequence as shown in SEQ ID NO: 47 by addition, deletion, or substitution of 1 or a plurality of nucleotides (which is a nucleotide sequence derived from the nucleotide sequence as shown in SEQ ID NO: 47 preferably by substitution, deletion, and/or addition, and more preferably by deletion and/or addition, of 1 or a plurality of nucleotides at either or both of the 5′ terminus and the 3′ terminus) and encoding a polypeptide having activity as SapF;
(9-5G) a nucleotide sequence having 80% or higher, preferably 85% or higher, and more preferably 90% or higher, 95% or higher, 97% or higher, 98% or higher, or 99% or higher sequence identity to the nucleotide sequence as shown in SEQ ID NO: 47 and encoding a polypeptide having activity as SapF;
(9-5H) a partial nucleotide sequence of any of the nucleotide sequences (9-5E) to (9-5G) encoding an amino acid sequence of a polypeptide having activity as SapF;
(9-5I) a nucleotide sequence derived from any of the nucleotide sequences (9-5E) to (9-5H) by introduction of silent mutation (nucleotide substitution that does not alter amino acids to encode);
(9-5J) a nucleotide sequence encoding the amino acid sequence of any of the polypeptides (9-5A) to (9-5D); and
(9-5K) a nucleotide sequence comprising, as an exon sequence, any of the nucleotide sequences (9-5E) to (9-5J) and one or more intron sequences therein.

In (9-5F) above, the term “a plurality of” refers to, for example, 2 to 60, 2 to 45, 2 to 30, 2 to 21, 2 to 15, 2 to 6, or 2 or 3.

In (9-5G) above, “sequence identity” is a value determined by aligning 2 nucleotide sequences, introducing gaps, according to need, so as to maximize the extent of nucleotide consistency therebetween, and determining a percentage (%) of identical nucleotides based on the total number of nucleotides in the nucleotide sequence as shown in SEQ ID NO: 47.

### The Microbial Strain of the Present Invention

One or more embodiments of the present invention relate to a microbial strain capable of overproduction of γ-glutamylcysteine, bis-γ-glutamylcystine, γ-glutamylcystine, reduced glutathione, and/or oxidized glutathione, which has the gene modification [1]:

enhanced expression of a gene encoding serine-O-acetyltransferase (EC:2.3.1.30).

The microbial strain is capable of efficient production of γ-glutamylcysteine, bis-γ-glutamylcystine, γ-glutamylcystine, reduced glutathione, and/or oxidized glutathione by fermentation.

The term “a microbial strain capable of overproduction of γ-glutamylcysteine, bis-γ-glutamylcystine, γ-glutamylcystine, reduced glutathione, and/or oxidized glutathione” used herein refers to a microbial strain that has the ability of producing γ-glutamylcysteine, bis-γ-glutamylcystine, γ-glutamylcystine, reduced glutathione, and/or oxidized glutathione higher than that of the host strain (the wild-type or parent strain) before introduction of a given gene modification.

It is preferable that the microbial strain further have the gene modification [2] and at least one gene modification selected from among the gene modifications [3] and [4]:
disruption of a gene encoding γ-glutamyltransferase (EC:3.4.19.13); and
enhanced expression of a gene encoding glutamate-cysteine ligase (EC:6.3.2.2) and/or a gene encoding glutathione synthetase (EC:6.3.2.3); and/or
enhanced expression of a gene encoding bifunctional glutathione synthetase.

The microbial strain having the gene modification [2] and at least one gene modification selected from among the gene modifications [3] and [4] can produce γ-glutamylcysteine, bis-γ-glutamylcystine, γ-glutamylcystine, reduced glutathione, and/or oxidized glutathione in an amount larger than the amount thereof produced by a host strain having neither the gene modification [2] nor at least one gene modification selected from among the gene modifications [3] and [4]. Accordingly, such microbial strain can produce the substances mentioned above with particularly high efficiency by further having such gene modifications, in combination with the gene modification [1].

When the microorganism is used for production of γ-glutamylcysteine, bis-γ-glutamylcystine, and/or γ-glutamylcystine, it is preferable that the microorganism have the gene modification [3]. In such a case, the gene modification [3] is preferably enhanced expression of a gene encoding glutamate-cysteine ligase.

When the microorganism is used for production of reduced glutathione and/or oxidized glutathione, the microorganism may have either or both the gene modifications [3] and [4]. In such a case, the gene modification [3] may be enhanced expression of one of a gene encoding glutamate-cysteine ligase and a gene encoding glutathione synthetase. It is more preferable that expression of both the genes are enhanced.

The microbial strain more preferably has at least one gene modification selected from among the gene modifications [5], [6], [7], [8], and [9]:
disruption of a gene encoding tryptophanase (EC:4.1.99.1);
disruption of a gene encoding tripeptide peptidase (EC:3.4.11.4);
disruption of at least 1 gene encoding a protein involved in glutathione import;
disruption of a gene encoding glutathione reductase (EC: 1.8.1.7); and
enhanced expression of at least 1 gene encoding a protein involved in putrescine export.

The microbial strain having at least one gene modification selected from among the gene modifications [5], [6], [7], [8], and [9] can produce γ-glutamylcysteine, bis-γ-glutamylcystine, γ-glutamylcystine, reduced glutathione, and/or oxidized glutathione in an amount larger than the amount thereof produced by a host strain that does not have such gene modification. Accordingly, the microbial strain having such gene modification in combination with the gene modification [1] can produce the substances as mentioned above with particularly high efficiency. It is more preferable that the microbial strain have the gene modification [1], the gene modification [2], and at least 1 of the gene modifications [3] and [4].

According to a more preferable embodiment, the microbial strain has at least 2, more preferably at least 3, more preferably at least 4, and more preferably all of the gene modifications [5], [6], [7], [8], and [9].

The microorganisms serving as hosts for the microbial strain according to one or more embodiments of the present invention are as described above.

Preferable examples of genes to be subjected to disruption or enhanced expression in the microbial strain according to one or more embodiments of the present invention are as provided above.

Enhanced expression of a particular gene in the microbial strain according to one or more embodiments of the present invention is described.

The gene subjected to the enhanced expression as defined in [1], [3], [4], and [9] above may be referred to as an “expression-enhanced gene.” The microbial strain having at least one gene modification selected from among [1], [3], [4], and [9] encompasses both of the following conditions. When the host strain (the wild-type or parent strain) of the microbial strain inherently expresses the expression-enhanced gene, the expression level of the expression-enhanced gene is increased compared with the expression level thereof in the host strain. When the host strain does not inherently express the expression-enhanced gene, the capacity for expressing the expression-enhanced gene is given to the host strain.

The increased expression levels of the expression-enhanced genes can be achieved by, for example, replacing a promoter that regulates the expression of the expression-enhanced genes in genomic DNA of cells of a microbial strain with a stronger expression promoter or increasing the copy number of the expression-enhanced genes in cells of a microbial strain. When a genetically modified microbial strain prepared by introducing a gene modification to replace a first expression promoter with a second expression promoter that is different from the first expression promoter inherent to the host strain into the host strain inherently having the expression-enhanced genes, in order to regulate the expression of the expression-enhanced genes, has the ability to express the expression-enhanced genes at a higher level than in the host strain, the second expression promoter is a “stronger expression promoter.”

When a promoter for the expression-enhanced gene in genomic DNA of microbial cells is to be replaced with a stronger expression promoter, specific examples of preferable expression promoters include tac promoter, trc promoter, ompF promoter, ompA promoter, cysK promoter, and lpp promoter. SEQ ID NO: 6 shows an example of a nucleotide sequence comprising tac promoter ligated to the SD sequence. SEQ ID NO: 8 shows an example of a nucleotide sequence comprising trc promoter ligated to the SD sequence. SEQ ID NO: 10 shows an example of a nucleotide sequence comprising ompF promoter ligated to the SD sequence. SEQ ID NO: 18 shows an example of a nucleotide sequence comprising ompA promoter ligated to the SD sequence. SEQ ID NO: 19 shows an example of a nucleotide sequence comprising cysK promoter ligated to the SD sequence. SEQ ID NO: 20 shows an example of a nucleotide sequence comprising lpp promoter ligated to the SD sequence.

An inducible promoter may be used as an expression promoter. The expression promoter indicated above may be operably linked to an operator sequence to prepare an inducible promoter.

Examples of inducible promoters include isopropyl-β-thiogalactopyranoside (IPTG) inducible promoter, photoinducible promoter that induces gene expression under light application, araBAD promoter (arabinose inducible), rhaBAD promoter (rhamnose inducible), tet promoter (drug inducible), penP promoter (drug inducible), cspA promoter (low-temperature inducible promoter), and a promoter comprising, as an operator sequence, tetO or lacO operator, with IPTG inducible promoter, araBAD promoter, rhaBAD promoter, tet promoter, penP promoter, cspA promoter, or a promoter comprising, as an operator sequence, tetO or lacO operator being preferable.

Specific examples of IPTG inducible promoters include lacUV5 promoter, lac promoter, lacT5 promoter, lacT7 promoter, and T5 promoter, T7 promoter, and tac promoter each converted to be IPTG inducible by being operably linked to an operator sequence. Among various types of inducible promoters, IPTG inducible promoters are particularly preferable. Among IPTG inducible promoters, T5 promoter, T7 promoter, lacT5 promoter, lacT7 promoter, or tac promoter is particularly preferable.

As a promoter, a highly active promoter modified from an existing promoter with the use of various reporter genes can be used. For example, the -35 region and the -10 region in the promoter region may be made close to the consensus sequence so as to enhance promoter activity (WO 2000/018935). Examples of highly active promoters include various tac-like promoters (Katashkina JI et al., Russian Federation Patent application 2006134574). A method for evaluation of promoter strength and examples of strong promoters are described in, for example, the literature of Goldstein et al. (Prokaryotic promoters in biotechnology, Biotechnol. Annu. Rev., 1, 105-128, 1995).

In order to enhance the expression levels of the expression-enhanced genes, the copy number of the expression-enhanced genes in cells of a microbial strain can be increased by:
(A) introduction of an expression vector comprising the expression-enhanced genes into cells of a microbial strain; or
(B) introduction of the expression-enhanced genes into genomic DNA of cells of a microbial strain.

As an expression vector used in the embodiment (A) above, for example, a plasmid vector comprising the expression-enhanced genes can be used. It is preferable that an expression vector be capable of autonomous replication in cells of a microorganism. It is preferable that an expression vector comprise DNA encoding a particular protein and a promoter operably linked to a position where the DNA can be transcribed. It is preferable that an expression vector be capable of autonomous replication in cells of a microorganism and that an expression vector be recombinant DNA comprising a nucleotide sequence composed of a promoter, a ribosome binding sequence, a nucleotide sequence encoding the amino acid sequences of the one or more enzymes, and a transcription terminator sequence.

It is preferable that the microbial strain according to one or more embodiments of the present invention expressibly carry an expression vector comprising a nucleotide sequence encoding the expression-enhanced genes. When “the expression-enhanced genes can be expressed,” the expression-enhanced genes can be expressed constitutively, or the expression-enhanced genes can be induced to express.

Examples of preferable plasmid vectors include: pQEK1, pCA24N (DNA RESEARCH, 12, 191-299, 2005), pACYC177, pACYC184 (available from Nippon Gene Co., Ltd.), pQE30, pQE60, pQE70, pQE80, and pQE9 (available from Qiagen); pTipQC1 (available from Qiagen or Hokkaido System Science Co., Ltd.) and pTipRT2 (available from Hokkaido System Science Co., Ltd.); pBS vector, Phagescript vector, Bluescript vector, pNH8A, pNH16A, pNH18A, and pNH46A (available from Stratagene); ptrc99a, pKK223-3, pKK233-3, pDR540, and pRIT5 (available from Addgene); pRSF (available from MERCK); and pAC (available from Nippon Gene Co., Ltd.), pUCN18 (which can be prepared via modification of pUC18 (available from Takara Bio Inc.)), pSTV28 (available from Takara Bio Inc.), and pUCNT (WO 94/03613).

The expression vector preferably comprises a promoter that regulates transcription of the expression-enhanced genes and more preferably comprises an inducible promoter. Examples of preferable promoters are as provided above.

When an expression vector comprising the expression-enhanced genes is introduced into cells of a microbial strain, the copy number of the expression vector in the cells is preferably 2 or more, more preferably 3 or more, more preferably 5 or more, more preferably 10 or more, more preferably 15 or more, and more preferably 20 or more.

When the expression levels of the two or more expression-enhanced genes are to be increased in cells of a microbial strain, an expression vector may comprise the two or more genes. In such a case, the two or more genes may be positioned under the control of an expression promoter. Alternatively, each of the two or more genes may be included in expression vectors separately from each other.

When the expression-enhanced genes are to be introduced into genomic DNA of cells of a microbial strain in accordance with the embodiment (B) above, homologous recombination can be performed.

In the microbial strain according to one or more embodiments of the present invention, an extent of expression enhancement of the expression-enhanced genes (i.e., increase in the expression levels) is not particularly limited. The expression levels of the expression-enhanced genes can be represented as the amount of mRNAs corresponding to the expression-enhanced genes extracted from the cells. Such mRNA-based expression levels are preferably represented relative to the amount of mRNAs encoding adequate internal standard proteins. An example of the internal standard protein is a protein encoded by the hcaT gene known as a housekeeping gene. The amount of mRNA can be evaluated by, for example, Northern hybridization or RT-PCR (e.g., Molecular cloning, Cold Spring Harbor Laboratory Press, Cold spring Harbor, U.S.A., 2001). The mRNA-based expression levels of the expression-enhanced genes are preferably 150% or higher, 200% or higher, 500% or higher, 1,000% or higher, 1,200% or higher, 2,000% or higher, or 2,500% or higher, relative to the expression levels thereof in the host strain that is designated as 100%. While the upper limit of the mRNA-based expression levels of the expression-enhanced genes is not particularly limited, it can be, for example, 5,000% or lower or 3,000% or lower, relative to the expression levels thereof in the host strain that is designated as 100%.

An increase in the expression-enhanced gene expression levels can also be represented as an increase in the activity of a protein encoded by the expression-enhanced genes extracted from the cells. An increase in the protein activity can be verified by measuring the amount or activity of the protein. An increase in the amount of the protein encoded by the expression-enhanced genes can be verified via Western blotting using an antibody (Molecular cloning, Cold Spring Harbor Laboratory Press, Cold spring Harbor, U.S.A., 2001). In the microbial strain according to one or more embodiments of the present invention, the amount of the protein encoded by the expression-enhanced genes is preferably 150% or higher, 200% or higher, 500% or higher, 1,000% or higher, 1,200% or higher, 2,000% or higher, or 2,500% or higher, relative to the amount of the protein in the host strain that is designated as 100%. While the upper limit of the protein-based expression levels of the expression-enhanced genes is not particularly limited, it can be, for example, 5,000% or lower or 3,000% or lower, relative to the expression levels thereof in the host strain that is designated as 100%.

Subsequently, disruption of a particular gene in the microbial strain according to one or more embodiments of the present invention is described.

A gene to be disrupted in [2], [5], [6], [7], or [8] above may be referred to as a “gene to be disrupted.” When a gene is “disrupted” in [2], [5], [6], [7], or [8] above, activity of a protein encoded by the gene to be disrupted is lowered, compared with the activity of the host strain, and activity is completely quenched. The microbial strain according to one or more embodiments of the present invention is deprived of functions of the gene to be disrupted, or such functions are lowered in the microbial strain. In such microbial strain, specifically, the expression levels of mRNA, which is a transcription product of the gene to be disrupted, or a protein, which is a translation product thereof, are lowered, or mRNA, which is a transcription product of the gene to be disrupted, or a protein, which is a translation product thereof, would not normally function as mRNA or a protein.

Disruption of the gene to be disrupted can be achieved by, for example, artificial modification of the gene of the host strain. Such modification can be achieved by, for example, mutagenesis, gene recombination, or gene expression control using RNAi.

Mutagenesis can be performed via ultraviolet application or via treatment using a common agent causing mutation, such as N-methyl-N′-nitro-N-nitrosoguanidine (MNNG), ethyl methanesulfonate (EMS), or methyl methanesulfonate (MMS).

Gene recombination can be performed in accordance with a known technique (e.g., FEMS Microbiology Letters 165, 1998, 335-340, JOURNAL OF BACTERIOLOGY, December 1995, pp. 7171-7177, Curr. Genet., 1986,10 (8): pp. 573-578, or WO 98/14600).

A gene encoding a particular protein described in [2], [5], [6], [7], or [8] indicates, in addition to a coding region of the amino acid sequence of each protein, its expression control sequence (e.g., a promoter sequence), an exon sequence, an intron sequence, or the like without distinguishing them from each other. When an expression control sequence is to be modified, preferably one or more nucleotides, more preferably two or more nucleotides, and particularly preferably 3 or more nucleotides in the expression control sequence are modified.

Disruption of the gene to be disrupted is more preferably disruption of the gene to be disrupted in the genomic DNA of the microbial strain. The disruption of the gene to be disrupted may be disruption of a part or the whole of the expression control sequence or disruption of a part or the whole of the coding region of the amino acid sequence of the protein. The term “disruption” used herein refers to deletion or damage, with “deletion” being preferable.

The entire gene, including upstream and downstream sequences of the gene to be disrupted, may be deleted in the genomic DNA of the host strain. When a part or the whole of the coding region of the amino acid sequence of the protein encoded by the gene to be disrupted is to be deleted, the coding region in either of the N-terminal region, the internal region, the C-terminal region, and other regions may be deleted, provided that protein activity can be lowered. In general, the gene can be inactivated with certainty by deletion of a longer region. The reading frames of the upstream and downstream sequences of the region to be deleted are preferably inconsistent. A preferable embodiment is directed to a microbial strain comprising deletion of a region consisting of a number of nucleotides that is preferably at least 50%, more preferably at least 60%, more preferably at least 70%, more preferably at least 80%, more preferably at least 90%, and more preferably 100% of the total number of nucleotides constituting at least a part of the coding region of the amino acid sequence and/or the expression control sequence of the gene to be disrupted of genomic DNA, such as the coding region and/or the expression control sequence. It is particularly preferable that the microbial strain comprise disruption of a region from the start codon to the stop codon of the gene to be disrupted in genomic DNA.

Other examples of disruption of the gene to be disrupted to lower the protein activity include damage of the gene to be disrupted, such as introduction of amino acid substitution (missense mutation), introduction of a stop codon (nonsense mutation), and introduction of frameshift mutation via addition or deletion of 1 or 2 nucleotides into the amino acid sequence coding region of the gene to be disrupted in genomic DNA.

Disruption of the gene to be disrupted to lower the protein activity can be achieved by, for example, insertion of another sequence into the expression control sequence or the amino acid sequence coding region of the gene to be disrupted in genomic DNA. While another sequence may be inserted into any region of the gene, the gene can be inactivated with certainty via insertion of a longer sequence. It is preferable that reading frames of upstream and downstream sequences of the site of insertion be inconsistent. While “another sequence” is not particularly limited as long as functions of the protein to be encoded are lowered or quenched, examples thereof include a marker gene and a gene useful for production of a target substance, such as glutathione.

Disruption of the gene to be disrupted in genomic DNA can be achieved by, for example, preparing an inactive gene by modifying the gene to be disrupted so as not to produce a protein that normally functions, transforming the host strain with recombinant DNA containing the inactive gene, and causing homologous recombination between the inactive gene and a gene in genomic DNA to substitute the gene in genomic DNA with the inactive gene. In such a case, a marker gene may be incorporated into recombinant DNA in accordance with traits of hosts, such as auxotrophic properties. Thus, a procedure of interest is easily performed. The recombinant DNA may be linearized via cleavage with restriction enzymes, so that a strain comprising recombinant DNA integrated into genomic DNA can be efficiently obtained. If a protein encoded by the inactive gene is generated, a conformation thereof would be different from that of a wild-type protein, and functions thereof would be lowered or quenched.

For example, microorganisms may be transformed with linear DNA comprising an arbitrary sequence and, at both ends of the arbitrary sequence, upstream and downstream sequences of the target site of substitution (typically a part of or the entire gene to be disrupted) in genomic DNA or linear DNA comprising upstream and downstream sequences of the target site of substitution in genomic DNA directly ligated to each other to cause homologous recombination in regions upstream and downstream of the target site of substitution in genomic DNA of the host strain. Thus, the target site of substitution can be substituted with the sequence of the linear DNA in a single step. The arbitrary sequence may comprise, for example, a marker gene sequence. A marker gene may be removed later, according to need. When a marker gene is to be removed, sequences for homologous recombination may be added to both ends of the marker gene, so as to efficiently remove the marker gene.

Whether or not the gene to be disrupted has been disrupted in the microbial strain can be verified based on a lowering in the activity of the protein encoded by the gene to be disrupted. The lowering in protein activity can be verified by assaying the amount or activity of the protein.

A lowering in the transcription level of the gene to be disrupted can be verified by comparing the amount of mRNA transcribed from the gene of interest with the amount of mRNA of the host strain. The amount of mRNA can be evaluated by, for example, Northern hybridization or RT-PCR (e.g., Molecular cloning, Cold Spring Harbor Laboratory Press, Cold spring Harbor, U.S.A., 2001). It is preferable that the amount of mRNA be lowered to, for example, 50% or lower, 20% or lower, 10% or lower, 5% or lower, or 0% of the amount of mRNA in the host strain.

A lowering in the amount of a protein encoded by the gene to be disrupted can be verified via Western blotting using an antibody (Molecular cloning, Cold Spring Harbor Laboratory Press, Cold spring Harbor, U.S.A., 2001). In the microbial strain according to one or more embodiments of the present invention, the amount of the protein encoded by the gene to be disrupted is preferably lowered to, for example, 50% or lower, 20% or lower, 10% or lower, 5% or lower, or 0% of the amount of the protein in the host strain.

### The Method for Producing Γ-Glutamylcysteine, Bis-γ-Glutamylcystine, Γ-Glutamylcystine, Reduced Glutathione, and/or Oxidized Glutathione According to the Present Invention

Further one or more embodiments of the present invention relate to a method for producing γ-glutamylcysteine, bis-γ-glutamylcystine, γ-glutamylcystine, reduced glutathione, and/or oxidized glutathione comprising culturing the microbial strain according to one or more embodiments of the present invention.

According to such a method, γ-glutamylcysteine, bis-γ-glutamylcystine, γ-glutamylcystine, reduced glutathione, and/or oxidized glutathione can be produced at low cost. According to such a method, the target substance can be produced at high productivity. According to an embodiment of the method, the yield of the target substance relative to the sugar raw materials supplied to the medium is high (i.e., the high sugar yield). According to another embodiment, the target substance can be secreted in the medium at high concentration.

When the method is to produce reduced glutathione and/or oxidized glutathione, the microbial strain used preferably has the gene modification [1], the gene modification [2], and at least one gene modification selected from among the gene modifications [3] and [4]. The gene modification [3] is preferably enhanced expression of both the gene encoding glutamate-cysteine ligase and the gene encoding glutathione synthetase.

When the method is to produce γ-glutamylcysteine, bis-γ-glutamylcystine, and/or γ-glutamylcystine, the microbial strain preferably has the gene modification [1], the gene modification [2], and at least one gene modification selected from among the gene modifications [3] and [4]. The gene modification [3] is preferably enhanced expression of the gene encoding glutamate-cysteine ligase.

The microbial strain according to one or more embodiments of the present invention can be cultured in an adequate medium. The medium may be either a synthetic or natural medium, provided that it contains nutrients necessary for the growth of the microorganism used in the present invention, such as carbon sources, nitrogen sources, inorganic salts, and vitamins, and for the biosynthesis of the target substance. Use of M9 medium is preferable.

Any carbon sources can be used, provided that carbon sources are assimilated by the microorganism used. Examples of carbon sources include saccharides, such as glucose and fructose, alcohols, such as ethanol and glycerol, and organic acids, such as acetic acid.

Examples of nitrogen sources include nitrogen compounds, such as ammonia, ammonium salt such as ammonium sulfate, and amine, and natural nitrogen sources, such as peptone and soybean hydrolysate.

Examples of inorganic salts include potassium phosphate, magnesium sulfate, sodium chloride, ferrous sulfate, and potassium carbonate.

Examples of vitamins include biotin and thiamine. Optionally, substances required for the growth of the microbial strain according to one or more embodiments of the present invention (e.g., auxotrophic amino acids in the case of amino acid auxotrophic microbial strain) can be added.

The medium is supplemented with at least one of a sulfur source and glycine and it is preferably supplemented with both of the sulfur source and glycine. For example, glycine can be added to a medium at 100 mM to 2000 mM, and preferably 400 mM to 1200 mM. The sulfur source can be added to the medium at, for example, 100 mM to 2000 mM, and preferably 400 mM to 1200 mM.

As sulfur sources, one or more types of inorganic sulfur compounds, such as sulfuric acid, thiosulfuric acid, sulfurous acid, hyposulfite, sulfide, or a salt thereof can be added. Sulfuric acid, thiosulfuric acid, sulfurous acid, hyposulfite, or sulfide may be in a free form, salt, or a compound of any thereof. Examples of salts include, but are not particularly limited to, sodium salt, calcium salt, ammonium salt, and potassium salt.

Glycine may be in a free form, salt, or a compound of any thereof. Examples of salts include, but are not particularly limited to, sulfate and hydrochloride.

Sulfur sources and/or glycine can be added to a medium when the culture is initiated or during culture. Sulfur sources and/or glycine may be added to the medium simultaneously, continuously, or intermittently.

Sulfur sources and/or glycine may be contained in the medium throughout the culture period or in a part of the culture period. For example, it is not necessary that the amount of sulfur sources and glycine to be added is within the range described above throughout the stage at which the target peptides are produced and accumulated. Sulfur sources and/or glycine may be added to the medium in a manner such that the content would be within the range described above during culture. With the elapse of time, the content of sulfur sources and/or glycine may be lowered. Sulfur sources and/or glycine may further be added continuously or intermittently. The concentration of medium components other than sulfur sources and/or glycine may vary during culture, and such medium components may further be added.

Culture is preferably carried out under aerobic conditions, such as via shake culture or aeration-agitation culture. Culture is performed at 20° C. to 50° C., preferably at 20° C. to 42° C., and more preferably at 28° C. to 38° C. At the time of culture, a pH is 5 to 9, and preferably 6 to 7.5. A culture period is 3 hours to 5 days, and preferably 5 hours to 3 days.

Target substances accumulated in the culture product can be collected in accordance with a conventional purification method. After the completion of culture, for example, bacteria or solids are removed from the culture product via centrifugation, and the target substances can be collected via ion exchange, concentration, or crystal fractionation.

### EXAMPLES

Hereafter, the present invention is described in greater detail with reference to the examples, although the present invention is not limited to these examples.

Genetic engineering described below can be performed with reference to Molecular Cloning (Cold Spring Harbor Laboratory Press, 1989). Enzymes, cloning hosts, and materials used for genetic engineering may be purchased from commercial providers and used in accordance with the instructions. The enzymes are not particularly limited, provided that they can be used for genetic engineering.

### Analysis of Glutathione Concentration in Culture Solution

The glutathione concentration in the culture solution was determined by high-performance liquid chromatography (HPLC, Shimadzu Corporation).

HPLC conditions are as described below.

Column: Develosil ODS-HG-3 4.6 mm × 250 mm (Nomura Chemical Co., Ltd.)

Mobile phase: A solution of 30.5 g of potassium dihydrogen-phosphate and 18 g of sodium heptane sulfonate in 4.5 liters of distilled water was prepared, a pH of the solution was adjusted to 3 with phosphoric acid, 250 ml of methanol was added thereto, and a pH of the solution was readjusted to 3 with phosphoric acid.
Flow rate: 1 ml/min
Detection: UV detector, λ = 210 nm
Column temperature: 40° C.
Amount of injection: 10 µl

When analyzing the glutathione concentration in the culture solution, cells were removed via centrifugation, and the supernatant was allowed to pass through a syringe filter (φ = 0.2 µm, Advantech Co., Ltd.) to obtain a culture supernatant. The culture supernatant was diluted to 10-fold with distilled water and the resultant was then subjected to HPLC.

(Production Example 1) Preparation of BW25113Δggt strain

At the outset, a plasmid vector for disrupting the ggt (y-glutamyltransferase) gene (SEQ ID NO: 23) was prepared. A DNA fragment (SEQ ID NO: 1) comprising the upstream sequence and the downstream sequence of the ggt gene on the chromosome was obtained by PCR using synthetic oligo DNA. The resulting fragment was digested withXbaI and HindIII, the temperature-sensitive plasmid pTH18cs1 (GenBank Accession Number: AB019610, Hashimoto-Gotoh, T., Gene, 241, 185-191, 2000) was digested withXbaI and HindIII, and the digested fragments were ligated to each other with the aid of Ligation high Ver. 2 (Toyobo Co., Ltd.) to obtain the plasmid vector, pTH18cs1-ggt-UD.

Subsequently, the BW25113Δggt strain was prepared using pTH18cs1-ggt-UD. pTH18cs1-ggt-UD was introduced into the E. coli BW25113 strain via electroporation, applied to an LB agar plate containing chloramphenicol at 10 µg/ml, and cultured at 30° C. to obtain transformants. The resulting transformants were shake-cultured in an LB liquid medium containing chloramphenicol at 10 µg/ml at 30° C. overnight, the culture solution was applied to an LB agar plate containing chloramphenicol at 10 µg/ml, and culture was performed at 42° C. to obtain transformants. The resulting transformants were cultured in an LB liquid medium at 42° C. overnight and applied to an LB agar plate to obtain colonies. The resulting colonies were replica-plated to an LB agar plate and an LB agar plate containing chloramphenicol at 10 µg/ml, and chloramphenicol-sensitive transformants were selected. The selected transformants were analyzed by PCR and using a DNA sequencer to isolate a strain having deletion of a region from the start codon to the stop codon of the ggt gene on the chromosome. This gene-disrupted strain was designated as the BW25113Δggt strain.

The BW25113Δggt strain is derived from the E. coli BW25113 host strain, and it has deletion of a region from the start codon to the stop codon of the ggt gene on the chromosome.

### (Production Example 2) Preparation of BW25113ΔggtΔpepT Strain

At the outset, a plasmid vector for disrupting the pepT (tripeptide peptidase) gene (SEQ ID NO: 25) was prepared. A DNA fragment (SEQ ID NO: 2) comprising the upstream sequence and the downstream sequence of the pepT gene on the chromosome was obtained by PCR using synthetic oligo DNA. The resulting fragment was digested withXbaI and HindIII, pTH18cs1 was digested withXbaI and HindIII, and the digested fragments were ligated to each other with the aid of Ligation high Ver. 2 to obtain the plasmid vector, pTH18cs1-pepT-UD.

Subsequently, a strain derived from a host strain; i.e., the BW25113Δggt strain prepared in Production Example 1, and having deletion of a region from the start codon to the stop codon of the pepT gene on the chromosome was isolated using pTH18cs1-pepT-UD in the same manner as in Production Example 1. This gene-disrupted strain was designated as the BW25113ΔggtΔpepT strain.

The BW25113ΔggtΔpepT strain is derived from the E. coli BW25113 host strain, and it has deletion of a region from the start codon to the stop codon of the ggt gene and that of the pepT gene on the chromosome.

### (Production Example 3) Preparation of BW25113ΔggtΔpepTΔgor Strain

At the outset, a plasmid vector for disrupting the gor (glutathione reductase) gene (SEQ ID NO: 27) was prepared. A DNA fragment (SEQ ID NO: 3) comprising the upstream sequence and the downstream sequence of the gor gene on the chromosome was obtained by PCR using synthetic oligo DNA. The resulting fragment was digested withXbaI and HindIII, pTH18cs1 was digested withXbaI and HindIII, and the digested fragments were ligated to each other with the aid of Ligation high Ver. 2 to obtain the plasmid vector, pTH18cs1-gor-UD.

Subsequently, a strain derived from a host strain; i.e., the BW25113ΔggtΔpepT strain prepared in Production Example 2, and having deletion of a region from the start codon to the stop codon of the gor gene on the chromosome was isolated using pTH18cs1-gor-UD in the same manner as in Production Example 1. This gene-disrupted strain was designated as the BW25113ΔggtΔpepTΔgor strain.

### (Production Example 4) Preparation of BW25113ΔggtΔpepTΔgorΔyliABCD Strain

At the outset, a plasmid vector for disrupting the yliABCD gene on the chromosome that forms an operon comprising the yliA (the glutathione transport system ATP-binding protein) gene (SEQ ID NO: 29), the yliB (the glutathione transport system substrate-binding protein) gene (SEQ ID NO: 31), the yliC (the glutathione transport system permease protein) gene (SEQ ID NO: 33), and the yliD (the glutathione transport system permease protein) gene (SEQ ID NO: 35) was prepared. A DNA fragment (SEQ ID NO: 4) comprising the upstream sequence of the yliA gene and the downstream sequence of the yliD gene on the chromosome was obtained by PCR using synthetic oligo DNA. The resulting fragment was digested with XbaI and HindIII, pTH18cs1 was digested withXbaI and HindIII, and the digested fragments were ligated to each other with the aid of Ligation high Ver. 2 to obtain the plasmid vector, pTH1 8cs1-yliABCD-UD.

Subsequently, a strain derived from a host strain; i.e., the BW25113ΔggtΔpepTΔgor strain prepared in Production Example 3, and having deletion of a region from the start codon to the stop codon of the yliABCD gene on the chromosome was isolated using pTH18cs1-yliABCD-UD in the same manner as in Production Example 1. This gene-disrupted strain was designated as the BW25113ΔggtΔpepTΔgorΔyliABCD strain.

### (Production Example 5) Preparation of BW25113ΔggtΔpepTΔgorΔyliABCDΔtnaA Strain

At the outset, a plasmid vector for disrupting the tnaA (tryptophanase) gene (SEQ ID NO: 37) was prepared. A DNA fragment (SEQ ID NO: 5) comprising the upstream sequence and the downstream sequence of the tnaA gene on the chromosome was obtained by PCR using synthetic oligo DNA. The resulting fragment was digested withXbaI and HindIII, pTH18cs1 was digested withXbaI and HindIII, and the digested fragments were ligated to each other with the aid of Ligation high Ver. 2 to obtain the plasmid vector, pTH18cs1-tnaA-UD.

Subsequently, a strain derived from a host strain; i.e., the BW25113ΔggtΔpepTΔgorΔyliABCD strain prepared in Production Example 4, and having deletion of a region from the start codon to the stop codon of the tnaA gene on the chromosome was isolated using pTH18cs1-tnaA-UD in the same manner as in Production Example 1. This gene-disrupted strain was designated as the BW25113ΔggtΔpepTΔgorΔyliABCDΔtnaA strain.

### (Production Example 6) Preparation of BW25113ΔggtΔpepTΔgorΔyliABCDΔtnaA Ptac-sapABCDF Strain

At the outset, the tac promoter and the SD sequence (SEQ ID NO: 6) were introduced into an upstream region of the sapABCDF gene on the chromosome that forms an operon comprising the sapA (a substrate-binding protein of the cationic peptide transport system) gene (SEQ ID NO: 39), the sapB (a permease protein of the cationic peptide transport system) gene (SEQ ID NO: 41), the sapC (a permease protein of the cationic peptide transport system) gene (SEQ ID NO: 43), the sapD (an ATP-binding protein of the cationic peptide transport system) gene (SEQ ID NO: 45), and the sapF (an ATP-binding protein of the cationic peptide transport system) gene (SEQ ID NO: 47) to prepare a plasmid vector for enhancing the expression levels of such genes. A DNA fragment (SEQ ID NO: 7) comprising the upstream sequence of the sapA gene, the tac promoter, the SD sequence, and a 500-bp sequence from the start codon of the sapA gene on the chromosome was obtained by PCR using synthetic oligo DNA. The resulting fragment was digested with BamHI and HindIII, pTH18cs1 was digested with BamHI and HindIII, and the digested fragments were ligated to each other with the aid of Ligation high Ver. 2 to obtain the plasmid vector, pTH18cs1-Ptac-sapA-UD.

Subsequently, a strain derived from a host strain; i.e., the BW25113ΔggtΔpepTΔgorΔyliABCDΔtnaA strain prepared in Production Example 5, by insertion of the tac promoter and the SD sequence into an upstream region of the sapA gene on the chromosome was isolated using pTH18cs1-Ptac-sapA-UD in the same manner as in Production Example 1. This strain was designated as the BW25113ΔggtΔpepTΔgorΔyliABCDΔtnaA Ptac-sapABCDF strain.

### (Production Example 7) Preparation of BW25113ΔggtΔpepTΔgorΔyliABCDΔtnaA Ptac-sapABCDF Ptrc-cysE Strain

At the outset, the trc promoter and the SD sequence (SEQ ID NO: 8) were introduced into an upstream region of the cysE (serine-O-acetyltransferase) gene (SEQ ID NO: 21) on the chromosome to prepare a plasmid vector for enhancing the expression levels of such genes. A DNA fragment (SEQ ID NO: 9) comprising the upstream sequence of the cysE gene, the trc promoter, the SD sequence, and a 500-bp sequence from the start codon of the cysE gene on the chromosome was obtained by PCR using synthetic oligo DNA. The resulting fragment was digested withXbaI and HindIII, pTH18cs1 was digested withXbaI and HindIII, and the digested fragments were ligated to each other with the aid of Ligation high Ver. 2 to obtain the plasmid vector, pTH18cs1-Ptrc-cysE-UD.

Subsequently, a strain derived from a host strain; i.e., the BW25113ΔggtΔpepTΔgorΔyliABCDΔtnaA Ptac-sapABCDF strain prepared in Production Example 6, by insertion of the trc promoter and the SD sequence into an upstream region of the cysE gene on the chromosome was isolated using pTH18cs1-Ptrc-cysE-UD in the same manner as in Production Example 1. This strain was designated as the BW25113ΔggtΔpepTΔgorΔyliABCDΔtnaA Ptac-sapABCDFPtrc-cysE strain.

### (Production Example 8) Preparation of BW25113ΔggtΔpepTΔgorΔyliABCDΔtnaA Ptac-sapABCDF PompFcysE Strain

At the outset, the ompF promoter and the SD sequence (SEQ ID NO: 10) were introduced into an upstream region of the cysE gene (SEQ ID NO: 21) on the chromosome to prepare a plasmid vector for enhancing the expression levels of such genes. A DNA fragment (SEQ ID NO: 11) comprising the upstream sequence of the cysE gene, the ompF promoter, the SD sequence, and a 500-bp sequence from the start codon of the cysE gene on the chromosome was obtained by PCR using synthetic oligo DNA. The resulting fragment was digested with XbaI and HindIII, pTH18cs1 was digested withXbaI and HindIII, and the digested fragments were ligated to each other with the aid of Ligation high Ver. 2 to obtain the plasmid vector, pTH18cs1-PompF-cysE-UD.

Subsequently, a strain derived from a host strain; i.e., the BW25113ΔggtΔpepTΔgorΔyliABCDΔtnaA Ptac-sapABCDF strain prepared in Production Example 6, by insertion of the ompF promoter and the SD sequence into an upstream region of the cysE gene on the chromosome was isolated using pTH18cs1-PompF-cysE-UD in the same manner as in Production Example 1. This strain was designated as the BW25113ΔggtΔpepTΔgorΔyliABCDΔtnaA Ptac-sapABCDF PompF-cysE strain.

### (Production Example 9) Preparation of pQEK1-PT5-ABTd*-Term

At the outset, the pQEK1 vector as shown in SEQ ID NO: 12 was constructed from pQE-80L (QIAGEN) by replacing the drug-resistant marker with a tetracycline-resistant gene, so as to construct a vector for introducing a gene into E. coli. In addition, a lambda phage-derived terminator sequence was inserted into the HindIII locus of pQEK1 to construct the pQEK1-term vector as shown in SEQ ID NO: 13.

Subsequently, a DNA fragment (SEQ ID NO: 14) comprising the T5 promoter, the E. coli-derived gshA gene (SEQ ID NO: 55), and the Thiobacilus denitrificans-derived gshB gene (with V260A mutation) (SEQ ID NO: 51) was obtained by PCR using synthetic oligo DNA. The resulting fragment was ligated to a fragment obtained by digesting pQEK1-term with SpeI and HindIII using NEBuilder HiFi DNA Assembly Master Mix (New England Biolabs) to obtain pQEK1-PT5-ABTd*-term shown in SEQ ID NO: 15.

### (Production Example 10) Preparation of pQEK1-PT5-FSa-Term

A DNA fragment (SEQ ID NO: 16) comprising the T5 promoter and the Streptococcus agalactiae-derived gshF gene (SEQ ID NO: 53) was obtained by PCR using synthetic oligo DNA. The resulting fragment was ligated to a fragment obtained by digesting pQEK1-term with SpeI and HindIII using NEBuilder HiFi DNA Assembly Master Mix to obtain pQEK1-PT5-FSa-term shown in SEQ ID NO: 17.

### (Production Example 11) Preparation of BW25113ΔggtΔpepTΔgorΔyliABCDΔtnaA Ptac-sapABCDF/pQEK1-PT5-ABTd*-Term Strain

The pQEK1-PT5-ABTd*-term strain prepared in Production Example 9 was introduced into the BW25113ΔggtΔpepTΔgorΔyliABCDΔtnaA Ptac-sapABCDF strain prepared in Production Example 6 via electroporation, and the resultant was applied to an LB agar plate containing tetracycline at 20 µg/ml to select transformants. The selected transformants were subjected to PCR analysis to isolate a strain comprising pQEK1-PT5-ABTd*-term introduced thereinto. This strain was designated as the BW25113ΔggtΔpepTΔgorΔyliABCDΔtnaA Ptac-sapABCDF/pQEK1-PT5-ABTd*-term strain.

### (Production Example 12) Preparation of BW25113ΔggtΔpepTΔgorΔyliABCDΔtnaA Ptac-sapABCDF Ptrc-cysE/pQEK1 -PT5 -ABTd* -Term Strain

pQEK1-PT5-ABTd*-term prepared in Production Example 9 was introduced into the BW25113ΔggtΔpepTΔgorΔyliABCDΔtnaA Ptac-sapABCDF Ptrc-cysE strain prepared in Production Example 7 via electroporation, and the resultant was applied to an LB agar plate containing tetracycline at 20 µg/ml to select transformants. The selected transformants were subjected to PCR analysis to isolate a strain comprising pQEK1-PT5-ABTd*-term introduced thereinto. This strain was designated as the BW25113ΔggtΔpepTΔgorΔyliABCDΔtnaA Ptac-sapABCDF Pirc-cysElpQEFA -PT5 -ABTd* -term strain.

### (Production Example 13) Preparation of BW25113ΔggtΔpepTΔgorΔyliABCDΔtnaA Ptac-sapABCDF PompF-cysE/pQEK1-PT5-ABTd*-Term Strain

pQEK1-PT5-ABTd*-term prepared in Production Example 9 was introduced into the BW25113ΔggtΔpepTΔgorΔyliABCDΔtnaA Ptac-sapABCDF PompF-cysE strain prepared in Production Example 8 via electroporation, and the resultant was applied to an LB agar plate containing tetracycline at 20 µg/ml to select transformants. The selected transformants were subjected to PCR analysis to isolate a strain comprising pQEK1-PT5-ABTd*-term introduced thereinto. This strain was designated as the BW25113ΔggtΔpepTΔgorΔyliABCDΔtnaA Ptac-sapABCDF PompF-cysE/pQEK1-PT5-ABTd*-term strain.

### (Production Example 14) Preparation of BW25113ΔggtΔpepTΔgorΔyliABCDΔtnaA Ptac-sapABCDF/pQEK1-PT5-FSa-Term Strain

pQEK1-PT5-FSa-term prepared in Production Example 10 was introduced into the BW25113ΔggtΔpepTΔgorΔyliABCDΔtnaA Ptac-sapABCDF strain prepared in Production Example 6 via electroporation, and the resultant was applied to an LB agar plate containing tetracycline at 20 µg/ml to select transformants. The selected transformants were subj ected to PCR analysis to isolate a strain comprising pQEK1-PT5-FSa-term introduced thereinto. This strain was designated as the BW25113ΔggtΔpepTΔgorΔyliABCDΔtnaA Ptac-sapABCDF/pQEK1-PT5-FSa-term strain.

### (Production Example 15) Preparation of BW25113ΔggtΔpepTΔgorΔyliABCDΔtnaA Ptac-sapABCDFPtrc-cysE/pQEK1-PTS-FSa-Term Strain

pQEK1-PT5-FSa-term prepared in Production Example 11 was introduced into the BW25113ΔggtΔpepTΔgorΔyliABCDΔtnaA Ptac-sapABCDF strain prepared in Production Example 6 via electroporation, and the resultant was applied to an LB agar plate containing tetracycline at 20 µg/ml to select transformants. The selected transformants were subj ected to PCR analysis to isolate a strain comprising pQEK1-PT5-FSa-term introduced thereinto. This strain was designated as the BW25113ΔggtΔpepTΔgorΔyliABCDΔtnaA Ptac-sapABCDFPtrc-cysE/pQEK1-PT5-FSa-term strain.

### (Example 1) Production of Glutathione by Fermentation Using the BW25113ΔggtΔpepTΔgorΔyliABCDΔtnaA Ptac-sapABCDF Ptrc-cysE/pQEK1-PT5-ABTd*-Term Strain

The BW25113ΔggtΔpepTΔgorΔyliABCDΔtnaA Ptac-sapABCDFPtrc-cysE/pQEK1-PT5-ABTd*-term strain obtained in Production Example 12 was cultured under the conditions described below to produce GSH and GSSG. The strain obtained in Production Example 12 was inoculated into 5 ml of LB medium containing tetracycline at 20 µg/ml and shake-cultured therein at 300 rpm and 30° C. for 8 hours. The culture solution (1 ml) was inoculated into 100 ml of M9 medium (6 g/l disodium hydrogen-phosphate, 3 g/l potassium dihydrogen-phosphate, 0.5 g/l sodium chloride, 1 g/l ammonium chloride, 1 mM magnesium sulfate, 0.001 % thiamine-hydrochloric acid, 0.1 mM calcium chloride, 2% glucose) supplemented with 20 µg/ml tetracycline. After inoculation, the culture solution was cultured using a culture apparatus (Bio Jr.8, Able Corporation) at 34° C. and pH 6.5 with shaking at 1,000 rpm and aeration of 100 ml/min for 18 hours. The culture solution 18 hours after the initiation of culture (20 ml) was inoculated into 2 liters of M9 medium supplemented with 20 µg/ml tetracycline and then cultured using a culture apparatus (Bioneer-Neo, Marubishi Bioengineering Co., Ltd.) at 34° C. and pH 6.7 with shaking at 600 rpm and aeration of 4 l/min. During culture, a 50 w/v% glucose solution was added, according to need, so as to maintain the glucose concentration to 15 g/l or higher in the system. 0.1 mM isopropyl-P-thiogalactopyranoside was added 6 hours after the initiation of culture, and, at the same time, glycine and sodium sulfate were added to adjust the final concentration to 100 mM. An adequate amount of the culture solution was sampled 30 hours after the initiation of culture, and cells were separated from the supernatant via centrifugation. The supernatant was adequately diluted with distilled water, and GSH and GSSG were quantified by HPLC analysis. The results of quantification are shown in Table 1.

### (Example 2) Production of Glutathione by Fermentation Using the BW25113ΔggtΔpepTΔgorΔyliABCDΔtnaA Ptac-sapABCDF PompF-cysE/pQEK1-PT5-ABTd*-Term Strain

The BW25113ΔggtΔpepTΔgorΔyliABCDΔtnaAPtac-sapABCD-FpompF-cysE/pQEK1-PT5-ABTd*-term strain obtained in Production Example 13 was cultured under the same conditions as those in Example 1 to produce GSH and GSSG. The results are shown in Table 1.

### (Comparative Example 1) Production of Glutathione by Fermentation Using the BW25113ΔggtΔpepTΔgorΔyliABCDΔtnaA Ptac-sapABCDF/pQEK1-PT5-ABTd*-Term Strain

The BW25113ΔggtΔpepTΔgorΔyliABCDΔtnaA Ptac-sapABCDF/pQEK1-PT5-ABTd*-term strain obtained in Production Example 11 was cultured under the same conditions as those in Example 1 to produce GSH and GSSG. The results are shown in Table 1.

TABLE 1

| | Strain | GDH + GSSG (g/1) |
|---|---|---|
| Ex. 1 | BW25113ΔggtΔpepTΔgorΔyliABCDΔtnaA Ptac-sapABCDF Ptrc-cysE / pQEK1-PT5-ABTd*-term | 10.1 |
| Ex. 2 | BW25113ΔggtΔpepTΔgorΔyliABCDΔtnaA Ptac-sapABCDF PompF-cysE / pQEK1-PT5-ABTd*-term | 9.4 |
| Comp. Ex. 1 | BW25113ΔggtΔpepTΔgorΔyliABCDΔtnaA Ptac-sapABCDF / pQEK1-PT5-ABTd^{∗}-term | 8.2 |

### Examination

The results of Examples 1 and 2 and the results of Comparative Example 1 shown in Table 1 demonstrate that glutathione productivity (GSH + GSSG) is increased to a significant extent by enhanced expression of the cysE (serine-O-acetyltransferase) gene. This indicates that enhanced expression of the cysE gene is effective for glutathione production by fermentation.

### (Example 3) Production of Glutathione by Fermentation Using the BW25113ΔggtΔpepTΔgorΔyliABCDΔtnaA Ptac-sapABCDF Ptrc-cysE/pQEK1-PT5-FSa-Term Strain

The BW25113ΔggtΔpepTΔgorΔyliABCDΔtnaA Ptac-sapABCDF Ptrc-cysE/pQEk1 -PT5-FSa-term strain obtained in Production Example 15 was cultured under the same conditions as those in Example 1 to produce GSH and GSSG. The results are shown in Table 2.

### (Comparative Example 2) Production of Glutathione by Fermentation Using the BW25113ΔggtΔpepTΔgorΔyliABCDΔtnaA Ptac-sapABCDF/pQEK1-PT5-FSa-Term Strain

The BW25113ΔggtΔpepTΔgorΔyliABCDΔtnaA Ptac-sapABCDF/pQEK1-PT5-FSa-term strain obtained in Production Example 14 was cultured under the same conditions as those in Example 1 to produce GSH and GSSG. The results are shown in Table 2.

TABLE 2

| | Strain | GDH + GSSG (g/l) |
|---|---|---|
| Ex. 3 | BW25113ΔggtΔpepTΔgorΔyliABCDΔtnaA Ptac-sapABCDF Ptrc-cysE / pQEK1-PT5-FSa-term | 8.8 |
| Comp. Ex. 2 | BW25113ΔggtΔpepTΔgorΔyliABCDΔtnaA Ptac-sapABCDF / pQEK1-PT5-FSa-term | 6.9 |

### Examination

The results of Example 3 and the results of Comparative Example 2 shown in Table 2 demonstrate that glutathione productivity (GSH + GSSG) is increased to a significant extent by enhanced expression of the cysE (serine-O-acetyltransferase) gene. This indicates that enhanced expression of the cysE gene is effective for glutathione production by fermentation.

All publications, patents, and patent applications cited herein are incorporated herein by reference in their entirety.

1. A microbial strain capable of overproduction of γ-glutamylcysteine, bis-γ-glutamylcystine, γ-glutamylcystine, reduced glutathione, and/or oxidized glutathione, which has the gene modification [1]:
[1] enhanced expression of a gene encoding serine-O-acetyltransferase (EC:2.3.1.30).

2. The microbial strain according to claim 1, which has the gene modification [2] and at least one gene modification selected from among the gene modifications [3] and [4]:
[2] disruption of a gene encoding γ-glutamyltransferase (EC:3.4.19.13); and
[3] enhanced expression of a gene encoding glutamate-cysteine ligase (EC:6.3.2.2) and/or a gene encoding glutathione synthetase (EC:6.3.2.3); and/or
[4] enhanced expression of a gene encoding bifunctional glutathione synthetase.

3. The microbial strain according to claim 1 comprising at least one gene modification selected from among the gene modifications [5], [6], [7], [8], and [9]:
[5] disruption of a gene encoding tryptophanase (EC:4.1.99.1);
[6] disruption of a gene encoding tripeptide peptidase (EC:3.4.11.4);
[7] disruption of at least 1 gene encoding a protein involved in glutathione import;
[8] disruption of a gene encoding glutathione reductase (EC:1.8.1.7); and
[9] enhanced expression of at least 1 gene encoding a protein involved in putrescine export.

4. The microbial strain according to claim 1, which is a transformed bacterium.

5. The microbial strain according to claim 4, which is a transformed enteric bacterium.

6. The microbial strain according to claim 4, which is a transformed Gram-negative bacterium.

7. The microbial strain according to claim 4, which is a transformed E. coli strain.

8. A method for producing γ-glutamylcysteine, bis-γ-glutamylcystine, γ-glutamylcystine, reduced glutathione, and/or oxidized glutathione comprising culturing the microbial strain according to claim 1.


SEQUENCE LISTING (58 sequences)

SEQ ID NO: 1 - DNA, 1038 bp, Artificial
  ggt-UD
gcgcgctctagagtcgccgtttctcctctccggcagcatatgctgctgattcaacgaactatccggattacggttgctgtttaacatcccgccgttggtgttgggcagcatttgctgccgggcgggatttcgctcccccggctgtgactgcaacacccgctgagaattattgtttatctggttttctaaatgctgctgttgcaactgagtttgcgttttcagttgctgattcagcatccctttttgctggatttgctgagtctgcatctgggtttgcatccgctgctggctgggtatctgataccccggctggttagggttgttcagagtattaatgggctgtgcaaagccgacaaacggcaggagtgccgtaagaatcagaagtcgtttcatcgcgtatcctcctctgaagatatcctttaagtttactcgcttcccgacaaaacgatgattaattcagagttatataccaggcttagctggggttgccccttaatctctggagaataacgggttagcggccctcttcgtgggaagagggctattttgtcagggcaagccgaaggtagccttttttatttcgtaatcctgtagatattcttccagcggttttgccgttcccagagaatgaatttcaccactgtctttatcaataataaaaggtgcgttaccagctaagcgcgcggcctcatctccagtttcgagaaattctcgtgcttcgaaacagaaataccagccctggctaaagcgtccatgtagagtaatgacgaccgggagatctgcatcatcaaggtaatggttcgctttcgcgaatgcgtcgtgataagtaatcataattataataaatattattgttgagtgttatattattattcctgtgtgatacattgagcaaagacgcgttcatcttcgtcaaagatattattaaatgctggttttgaaaaaagttcatcggataagatatagtcacgaaataagtatctgctaatattttatgatttctttttcagaacgtcgaacgggattaagcttgcgcgc

SEQ ID NO: 2 - DNA, 1057 bp, Artificial
  pepT-UD
gcgcgctctagaacgtgggtgatgtcctcgttatccagcatgatgcgtccggaatcaacagtttccagacctgcaatcaggcgaagaacggttgttttaccgcagccagaagggccaagcagcgtgaggaactcgccattgttgatagtcagatccagctggggaatgacctctttaccatcaaagcatttgcgaattcccgccaattgcaccagcggtgaaagcgaactcggttgtttattcaattttttactctgtcccatgtaaacgcaacggatggcttaccgatgcggggtttgtggttaaccaccttggtgactcttaatgagggcggtaattctacggcaaaccgcttgaatcgccaatctttgttgtgaattactggcttagctttatattcattaaggtaatgctgataaatattcccgcttgcaggggtaaaagtgacctgacgcaatatttgtcttttcttgcttcttaataatgttgtcacaaaaagtgagggtgactacgcgaaaagggatgcggcatgtgatgccgcatccggcttaaatccaaacttacccttcgaagaaccaatacccgctattgaccagcgccgcgagcatcgcgaggaatgacggatcttccagcgcatcgccaaaattctccgcagtcagcgcaatgttgctggcgagtgcatccagtgccggacggtgcggggaatcgatcttctcaccattggcatacacgtcgtcgccaatgcgcaatacgcgcagaccacccaggcgcaccagcacttcaccttgtttcagcgcatcgtagatttcatccggctgataaggcggctctggcggcgcgatatccagttcatgacgtgactgggatataaactcgccaaaccattgcttaaagtgttccggctggttgatcaattcgagcatcatctcacgcagtttatccatctcttgcggcagaacatccgcaggatgagcgcgaggtggaacatccggatcgctgtagtagttgccgcccagttcacgttgcagcacataatcggcaaatcaagcttgcgcgc

SEQ ID NO: 3 - DNA, 1024 bp, Artificial
  gor-UD
gcgcgctctagagtcgaaaaagccgacggtttccagcagcttaaggccaaactgccgccggtttcccgccgtggtttaatccttatcgacccgccgtatgaaatgaaaactgactatcaagcggtggtcagcgggatagcagaaggttacaaacgtttcgccactggtatttacgcactgtggtatccggtggtgctgcgtcagcaaattaagcgcatgatccacgatctggaagcgaccggtattcgcaaaattctgcaaattgaactggcggtactgccagacagcgatcgccgtggcatgaccgcttccggcatgattgtgattaacccgccgtggaaactggaacaacagatgaataacgtgctgccgtggctgcacagcaaactggttccggcaggcaccgggcacgccaccgtaagctggatcgtgccggagtaattgcagccattgctggcacctattacgtctcgcgctacaatcgcggtaatcaacgataaggacactttgtcatgttaaagggctaagagtagtgtgctcttagcccttaattacgtttccgctatcagttcagaagctgaagcagaaagcggatcagttccagcagcgcaattaacgcccctagaacgatgattgctttatcaatcacccgttttctccatgcgatggagtgagaatgcatccgcttactcatccactgcctgtcacggcgcatgtctcattgttagataagaactctctcactccggccagagcatcagttaacggcaccacccgtacttctgaccaggactttgaaagcgtttatgcgcattgccagagtgaaaatgcctcagagctaactggataatcatacagtacatgcaggttataaaaccagcacgtccttgcaatagtttcagtatggtattagcattgatgcgttagatgatggctatctcactccagtcagagccaccaactcagggctggaaagtaaaaaaccgacgcaaagtcggtttttttacatccgaagcttgcgcgc

SEQ ID NO: 4 - DNA, 1474 bp, Artificial
  yliABCD-UD
cggtacccggggatcctctagagcggtggcgggcgttagtcatctgcgtaatccggttcttgccgcccggctggtgatggagcaaagcccgcatgtgatgatgattggcgaaggggcagaaaattttgcgtttgctcgtggcatggagcgcgtctcgccggagattttctccacgtctttgcgttatgaacaactactggcagcgcgcaaggaaggggcaaccgtcctcgaccatagcggtgcgccactggatgaaaaacagaaaatgggcaccgtgggggccgtggcgttggatttagacggcaatttggcggcagccacgtccacaggcggaatgaccaataaattacccggacgagttggcgatagtcccttagtgggtgccggatgctacgccaataacgccagtgtggcggtttcttgtaccggcacgggcgaagtcttcatccgcgcgctggcggcatatgacatcgccgcgttaatggattacggcggattaagtctcgcggaagcctgcgagcgggtagtaatggaaaaactccctgcgcttggcggtagcggtggcttaatcgctatcgaccatgaagggaatgtcgcgctaccgtttaacaccgaaggaatgtatcgcgcctggggctacgcaggcgatacgccaaccaccggtatctaccgtgaaaaaggggacaccgttgccacacagtgatgaacttgatgccggtaatttacgtttgaatattgcttgaaagggtaatcacctcacaggaaattattgccctaagcaagtgttgtaactttctgctgattttgtagaatcgggtaatttggttaaaaagccgcagcaagggacaatttttgcagcggcacagcgttcagatagttattttgttaaatgtattaacatgctgagtttatacgaaaagataaagataaggctgataattttatttttattggcagcactgtcatttattggtctttttttcatcattaactatcaactggtatcggagcgcgcggtaaaacgtgccgatagccgctttgaacttattcagaaaaacgttggctatttctttaaagatattgaacgttcggccctgacattaaaggactcactgtatttattaaaaaatacagaggagattcaacgcgccgtgattcttaaaatggaaatgatgccatttttagactcggtgggactggtacttgatgataataaatattatcttttttcgcggagggcgaatgataaaatcgttgtttatcatcaggaacaagtaaatggaccgcttgtcgacgagtcagggcgggttatttttgccgattttaacccatcgaaacgaccgtggtcggtggcttcagatgactctaacaacagctggaatccggcatacaattgctttgatcgtccgggtaaaaaatgtatctcttttacgctacacatcaacggcaaagcttactggccgtcgttttacaa

SEQ ID NO: 5 - DNA, 1038 bp, Artificial
  tnaA-UD
gcgcgctctagagcttcgcttcattgttaccactcctgttattcctcaaccctttttttaaacattaaaattcttacgtaatttataatctttaaaaaaagcatttaatattgctccccgaacgattgtgattcgattcacatttaaacaatttcagaatagacaaaaactctgagtgtaataatgtagcctcgtgtcttgcgaggataagtgcattatgaatatcttacatatatgtgtgacctcaaaatggttcaatattgacaacaaaattgtcgatcaccgcccttgatttgcccttctgtagccatcaccagagccaaaccgattagattcaatgtgatctatttgtttgctatatcttaattttgccttttgcaaaggtcatctctcgtttatttacttgttttagtaaatgatggtgcttgcatatatatctggcgaattaatcggtatagcagatgtaatattcacagggatcactgtaattaaaataaatgaaggattatgtattaatactacagagtggctataaggatgttagccactctcttaccctacatcctcaataacaaaaatagccttcctctaaaggtggcatcatgactgatcaagctgaaaaaaagcactctgcattttggggtgttatggttatagcaggtacagtaattggtggaggtatgtttgctttacctgttgatcttgccggtgcctggtttttctggggtgcctttatccttatcattgcctggttttcaatgcttcattccgggttattgttattagaagcaaatttaaattatcccgtcggctccagttttaacaccatcaccaaagatttaatcggtaacacctggaacattatcagcggtattaccgttgccttcgttctctatatcctcacttatgcctatatctctgctaatggtgcgatcattagtgaaacgatatcaatgaatttgggttatcacgctaatccacgtattgtcgggatctgcacagccattttcgttgccagcgtattgtaagcttgcgcgc

SEQ ID NO: 6 - DNA, 60 bp, Artificial
  Ptac
ttgacaattaatcatcggctcgtataatgtacgcaagttcacgtaaaaagggtatcgaca

SEQ ID NO: 7 - DNA, 1102 bp, Artificial
  Ptac-sapABCDF-UD
agctcggtacccggggatccgcgcagagcctgtcgcagaggctgaacgcacaccgctggcccactacttccagttactgctcacccgtttgatgaacaatgaagagatcagcgaagaagcccaacatgaaatggctgctgaagcggggattaatcccgtacgcattgatgagatcgcagagtttttgaatcaatggggtaatgagtaatgatttaattgcagacgatcggtcagtatcgttgtccggataaagcgttcacgccgcatctggctaacaatgacacgcactgcagcgtgctttaatgggtttctcacgatcgtaccgctctgctcctcgctaagttgttctgtgagttatagttatagagaaatcacgggtacgaatgttgaacagatggcgacgtcacctgttcaacgaccaaccacacggccctcattacgacccctgaacacaaacggtaatggcaaatgtcattaaatattagtaagatagcctctttacataaaatcccttaatattttgacaattaatcatcggctcgtataatgtacgcaagttcacgtaaaaagggtatcgacaatgcgccaggtattatcgtctcttttggtgattgctggacttgtgagtggtcaggcaatcgccgcgcctgaatctcccccgcatgctgatatccgcgacagcggttttgtctattgcgtcagcgggcaagtcaacacctttaacccatccaaagcgagcagtgggttaattgtcgatacccttgccgcccagttttatgatcgactgctggatgtcgatccctatacctatcgcctgatgccggaacttgccgaaagctgggaagtactcgacaacggcgcgacctatcgcttccacctgcgtcgcgatgttccgtttcaaaaaaccgactggtttactcccactcgtaaaatgaatgccgacgatgtggtgtttaccttccagcgaatttttgaccgcaacaacccgtggcataacgtcaacggcagcaacttcccctacttcgacagcctgcaatttgccgataacgtgaaaagcgtccgcaaactggataatcatacaagcttactggccgtcgtttta

SEQ ID NO: 8 - DNA, 52 bp, Artificial
  Ptrc
ttgacaattaatcatccggctcgtataatgtgtggtcacaaaggagatatac

SEQ ID NO: 9 - DNA, 1076 bp, Artificial
  Ptrc-cysE-UD
gcgcgctctagatggtgcggggatgtccgacggtatcggttttggtgcgaatgcgcgtacggcgctgatcacccgtgggctggctgaaatgtcgcgtcttggtgcggcgctgggtgccgaccctgccacctttatgggcatggcggggcttggcgatctggtgcttacctgtaccgacaaccagtcgcgtaaccgccgttttggcatgatgctcggtcagggcatggatgtacaaagcgcgcaggagaagattggtcaggtggtggaaggctaccgcaatacgaaagaagtccgcgaactggcgcatcgcttcggcgttgaaatgccaataaccgaggaaatttatcaagtattatattgcggaaaaaacgcgcgcgaggcagcattgactttactaggtcgtgcacgcaaggacgagcgcagcagccactaaccccagggaacctttgttaccgctatgacccggcccgcgcagaacgggccggtcattatctcatcgtgtggagtaagcattgacaattaatcatccggctcgtataatgtgtggtcacaaaggagatatacatgtcgtgtgaagaactggaaattgtctggaacaatattaaagccgaagccagaacgctggcggactgtgagccaatgctggccagtttttaccacgcgacgctactcaagcacgaaaaccttggcagtgcactgagctacatgctggcgaacaagctgtcatcgccaattatgcctgctattgctatccgtgaagtggtggaagaagcctacgccgctgacccggaaatgatcgcctctgcggcctgtgatattcaggcggtgcgtacccgcgacccggcagtcgataaatactcaaccccgttgttatacctgaagggttttcatgccttgcaggcctatcgcatcggtcactggttgtggaatcaggggcgtcgcgcactggcaatctttctgcaaaaccaggtttctgtgacgttccaggtcgatattcacccggcagcaaaaattggtcgcggtatcatgcttgaccacgcgacaggcatcgtcgttggtgaaacaagcttgcgcgc

SEQ ID NO: 10 - DNA, 300 bp, Escherichia coli
tataaggaaatcatataaatagattaaaattgctgtaaatatcatcacgtctctatggaaatatgacggtgttcacaaagttccttaaattttacttttggttacatattttttctttttgaaaccaaatctttatctttgtagcactttcacggtagcgaaacgttagtttgaatggaaagatgcctgcagacacataaagacaccaaactctcatcaatagttccgtaaatttttattgacagaacttattgacggcagtggcaggtgtcataaaaaaaaccatgagggtaataaata

SEQ ID NO: 11 - DNA, 1324 bp, Artificial
  PompF-cysE-UD
gcgcgctctagatggtgcggggatgtccgacggtatcggttttggtgcgaatgcgcgtacggcgctgatcacccgtgggctggctgaaatgtcgcgtcttggtgcggcgctgggtgccgaccctgccacctttatgggcatggcggggcttggcgatctggtgcttacctgtaccgacaaccagtcgcgtaaccgccgttttggcatgatgctcggtcagggcatggatgtacaaagcgcgcaggagaagattggtcaggtggtggaaggctaccgcaatacgaaagaagtccgcgaactggcgcatcgcttcggcgttgaaatgccaataaccgaggaaatttatcaagtattatattgcggaaaaaacgcgcgcgaggcagcattgactttactaggtcgtgcacgcaaggacgagcgcagcagccactaaccccagggaacctttgttaccgctatgacccggcccgcgcagaacgggccggtcattatctcatcgtgtggagtaagcatataaggaaatcatataaatagattaaaattgctgtaaatatcatcacgtctctatggaaatatgacggtgttcacaaagttccttaaattttacttttggttacatattttttctttttgaaaccaaatctttatctttgtagcactttcacggtagcgaaacgttagtttgaatggaaagatgcctgcagacacataaagacaccaaactctcatcaatagttccgtaaatttttattgacagaacttattgacggcagtggcaggtgtcataaaaaaaaccatgagggtaataaataatgtcgtgtgaagaactggaaattgtctggaacaatattaaagccgaagccagaacgctggcggactgtgagccaatgctggccagtttttaccacgcgacgctactcaagcacgaaaaccttggcagtgcactgagctacatgctggcgaacaagctgtcatcgccaattatgcctgctattgctatccgtgaagtggtggaagaagcctacgccgctgacccggaaatgatcgcctctgcggcctgtgatattcaggcggtgcgtacccgcgacccggcagtcgataaatactcaaccccgttgttatacctgaagggttttcatgccttgcaggcctatcgcatcggtcactggttgtggaatcaggggcgtcgcgcactggcaatctttctgcaaaaccaggtttctgtgacgttccaggtcgatattcacccggcagcaaaaattggtcgcggtatcatgcttgaccacgcgacaggcatcgtcgttggtgaaacaagcttgcgcgc

SEQ ID NO: 12 - DNA, 4130 bp, Artificial
  pQEK1
gcttaaaggatcctaactcgagagatccctgccatttggcggggatttgctagcttgaggcatcaaataaaacgaaaggctcagtcgaaagactgggcctttcgttttatctgttgtttgtcggtgaacgctctcctgagtaggacaaatccgccctctagattacgtgcagtcgatgataagctgtcaaacatgagaattgtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcggaatttcgggcagcgttgggtcctggccacgggtgcgcatgatctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagtcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcacctcgagggtaccgaattccccgggctgcagactagtgagctccatatgcccaa

SEQ ID NO: 13 - DNA, 4230 bp, Artificial
  pQEK1-term
gcttgactcctgttgatagatccagtaatgacctcagaactccatctggatttgttcagaacgctcggttgccgccgggcgttttttattggtgagaatagcttaaaggatcctaactcgagagatccctgccatttggcggggatttgctagcttgaggcatcaaataaaacgaaaggctcagtcgaaagactgggcctttcgttttatctgttgtttgtcggtgaacgctctcctgagtaggacaaatccgccctctagattacgtgcagtcgatgataagctgtcaaacatgagaattgtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcggaatttcgggcagcgttgggtcctggccacgggtgcgcatgatctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagtcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcacctcgagggtaccgaattccccgggctgcagactagtgagctccatatgcccaa

SEQ ID NO: 14 - DNA, 2691 bp, Artificial
  PT5-ABTd* insert
cgggctgcagactagtaaatcataaaaaatttatttgctttgtgagcggataacaattataatagattcaattgtgagcggataacaatttcacacagaattcaaaagatctaagaaggagatatacatatatgatcccggacgtatcacaggcgctggcctggctggaaaaacatcctcaggcgttaaaggggatacagcgtgggctggagcgcgaaactttgcgtgttaatgctgatggcacactggcaacaacaggtcatcctgaagcattaggttccgcactgacgcacaaatggattactaccgattttgcggaagcattgctggaattcattacaccagtggatggtgatattgaacatatgctgacctttatgcgcgatctgcatcgttatacggcgcgcaatatgggcgatgagcggatgtggccgttaagtatgccatgctacatcgcagaaggtcaggacatcgaactggcacagtacggcacttctaacaccggacgctttaaaacgctgtatcgtgaagggctgaaaaatcgctacggcgcgctgatgcaaaccatttccggcgtgcactacaatttctctttgccaatggcattctggcaagcgaagtgcggtgatatctcgggcgctgatgccaaagagaaaatttctgcgggctatttccgcgttatccgcaattactatcgtttcggttgggtcattccttatctgtttggtgcatctccggcgatttgttcttctttcctgcaaggaaaaccaacgtcgctgccgtttgagaaaaccgagtgcggtatgtattacctgccgtatgcgacctctcttcgtttgagcgatctcggctataccaataaatcgcaaagcaatcttggtattaccttcaacgatctttatgagtacgtagcgggccttaaacaggcaatcaaaacgccatcggaagagtacgcgaagattggtattgagaaagacggtaagaggctgcaaatcaacagcaacgtgttgcagattgaaaacgaactgtacgcgccgattcgtccaaaacgcgttacccgcagcggcgagtcgccttctgatgcgctgttacgtggcggcattgaatatattgaagtgcgttcgctggacatcaacccgttctcgccgattggtgtagatgaacagcaggtgcgattcctcgacctgtttatggtctggtgtgcgctggctgatgcaccggaaatgagcagtagcgaacttgcctgtacacgcgttaactggaaccgggtgatcctcgaaggtcgcaaaccgggtctgacgctgggtatcggctgcgaaaccgcacagttcccgttaccgcaggtgggtaaagatctgttccgcgatctgaaacgcgtcgcgcaaacgctggatagtattaacggcggcgaagcgtatcagaaagtgtgtgatgaactggttgcctgcttcgataatcccgatctgactttctctgcccgtatcttaaggtctatgattgatactggtattggcggaacaggcaaagcatttgcagaagcctaccgtaatctgctgcgtgaagagccgctggaaattctgcgcgaagaggattttgtagccgagcgcgaggcgtctgaacgccgtcagcaggaaatggaagccgctgataccgaaccgtttgcggtgtggctggaaaaacacgcctgataaggtacctaaggaggttacaatgaaactgctgttcgtcgttgatcccctggccagcttgaaaccgtacaaggatagctccgttgccatgatgcgcgcagcgtgtgctcgtggtcatgccgtgttcgcagcagaagcgcgcgcactgctggttcgtgatggggtggctcgttctcgtgcagatgctgtcgaaacgcgtggcgacgatgactggtatcgcgttaccgaaacgcgtgaatttgccttaaccgactttgatgcagtggtgatgcgcgcagatccgcccgttgacgtggattaccttctcgcgacgcacctgttaggcgtagccgaaaccaacggtgcacgtgtcctgaatcgcccgcgtgccttgcgcgatttcaacgagaaactggccattctggaatttccgcagtttgtcgcacctaccctggtaagtgcggacgcaaccgaaattgcccactttctggctgctcatgcggatatcatcgtcaaaccgctgactgagatgggtggctccggtgtgtttcgcctgggagttagcgatccgaatcggaacgcgattctggagacattaacccgtcgtggctctcgcccaatcatggctcagcggtatttgccagcgatctcagagggcgacaaacgcatcctgctgatcgacggcgaagtagtgccatgggccttggcgcgcattccgctgaccggtgaaactcgcgggaatcttgcggctggtggtacagcgcgcgcgcaaccgctcagtgaacgggatcgcgaaatcgccgaaacgattgccccttgggcacgcagccagggcattttccttgcgggcttagacgtgattggggattgcctcaccgagattaacgtgacatcgcctactggatttcaggagattaccgcccaatcgggccatgatgttgcggaccagttcattgcggcgatcgaacgcgcgacgcgtccggaatgataacatatgcccaagcttgactcctgttgatagatc

SEQ ID NO: 15 - DNA, 6866 bp, Artificial
  pQEK1-PT5-ABTd*-term
gcttgactcctgttgatagatccagtaatgacctcagaactccatctggatttgttcagaacgctcggttgccgccgggcgttttttattggtgagaatagcttaaaggatcctaactcgagagatccctgccatttggcggggatttgctagcttgaggcatcaaataaaacgaaaggctcagtcgaaagactgggcctttcgttttatctgttgtttgtcggtgaacgctctcctgagtaggacaaatccgccctctagattacgtgcagtcgatgataagctgtcaaacatgagaattgtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcggaatttcgggcagcgttgggtcctggccacgggtgcgcatgatctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagtcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcacctcgagggtaccgaatttcccgggctgcagactagtaaatcataaaaaatttatttgctttgtgagcggataacaattataatagattcaattgtgagcggataacaatttcacacagaattcaaaagatctaagaaggagatatacatatatgatcccggacgtatcacaggcgctggcctggctggaaaaacatcctcaggcgttaaaggggatacagcgtgggctggagcgcgaaactttgcgtgttaatgctgatggcacactggcaacaacaggtcatcctgaagcattaggttccgcactgacgcacaaatggattactaccgattttgcggaagcattgctggaattcattacaccagtggatggtgatattgaacatatgctgacctttatgcgcgatctgcatcgttatacggcgcgcaatatgggcgatgagcggatgtggccgttaagtatgccatgctacatcgcagaaggtcaggacatcgaactggcacagtacggcacttctaacaccggacgctttaaaacgctgtatcgtgaagggctgaaaaatcgctacggcgcgctgatgcaaaccatttccggcgtgcactacaatttctctttgccaatggcattctggcaagcgaagtgcggtgatatctcgggcgctgatgccaaagagaaaatttctgcgggctatttccgcgttatccgcaattactatcgtttcggttgggtcattccttatctgtttggtgcatctccggcgatttgttcttctttcctgcaaggaaaaccaacgtcgctgccgtttgagaaaaccgagtgcggtatgtattacctgccgtatgcgacctctcttcgtttgagcgatctcggctataccaataaatcgcaaagcaatcttggtattaccttcaacgatctttatgagtacgtagcgggccttaaacaggcaatcaaaacgccatcggaagagtacgcgaagattggtattgagaaagacggtaagaggctgcaaatcaacagcaacgtgttgcagattgaaaacgaactgtacgcgccgattcgtccaaaacgcgttacccgcagcggcgagtcgccttctgatgcgctgttacgtggcggcattgaatatattgaagtgcgttcgctggacatcaacccgttctcgccgattggtgtagatgaacagcaggtgcgattcctcgacctgtttatggtctggtgtgcgctggctgatgcaccggaaatgagcagtagcgaacttgcctgtacacgcgttaactggaaccgggtgatcctcgaaggtcgcaaaccgggtctgacgctgggtatcggctgcgaaaccgcacagttcccgttaccgcaggtgggtaaagatctgttccgcgatctgaaacgcgtcgcgcaaacgctggatagtattaacggcggcgaagcgtatcagaaagtgtgtgatgaactggttgcctgcttcgataatcccgatctgactttctctgcccgtatcttaaggtctatgattgatactggtattggcggaacaggcaaagcatttgcagaagcctaccgtaatctgctgcgtgaagagccgctggaaattctgcgcgaagaggattttgtagccgagcgcgaggcgtctgaacgccgtcagcaggaaatggaagccgctgataccgaaccgtttgcggtgtggctggaaaaacacgcctgataaggtacctaaggaggttacaatgaaactgctgttcgtcgttgatcccctggccagcttgaaaccgtacaaggatagctccgttgccatgatgcgcgcagcgtgtgctcgtggtcatgccgtgttcgcagcagaagcgcgcgcactgctggttcgtgatggggtggctcgttctcgtgcagatgctgtcgaaacgcgtggcgacgatgactggtatcgcgttaccgaaacgcgtgaatttgccttaaccgactttgatgcagtggtgatgcgcgcagatccgcccgttgacgtggattaccttctcgcgacgcacctgttaggcgtagccgaaaccaacggtgcacgtgtcctgaatcgcccgcgtgccttgcgcgatttcaacgagaaactggccattctggaatttccgcagtttgtcgcacctaccctggtaagtgcggacgcaaccgaaattgcccactttctggctgctcatgcggatatcatcgtcaaaccgctgactgagatgggtggctccggtgtgtttcgcctgggagttagcgatccgaatcggaacgcgattctggagacattaacccgtcgtggctctcgcccaatcatggctcagcggtatttgccagcgatctcagagggcgacaaacgcatcctgctgatcgacggcgaagtagtgccatgggccttggcgcgcattccgctgaccggtgaaactcgcgggaatcttgcggctggtggtacagcgcgcgcgcaaccgctcagtgaacgggatcgcgaaatcgccgaaacgattgccccttgggcacgcagccagggcattttccttgcgggcttagacgtgattggggattgcctcaccgagattaacgtgacatcgcctactggatttcaggagattaccgcccaatcgggccatgatgttgcggaccagttcattgcggcgatcgaacgcgcgacgcgtccggaatgataacatatgcccaa

SEQ ID NO: 16 - DNA, 2408 bp, Artificial
  PT5-Fsa insert
cgggctgcagactagtaaatcataaaaaatttatttgctttgtgagcggataacaattataatagattcaattgtgagcggataacaatttcacacagaattcaaaagatctaagaaggagatatacatatatgattattgaccgtctgctgcaacgctcccatagccatctgccgatcctgcaagccacctttggtctggaacgtgaatccctgcgcattcatcagccgacccagcgtgtggcccagacgccgcatccgaaaaccctgggctctcgcaactatcacccgtacatccaaacggattatagtgaaccgcagctggaactgattaccccgatcgccaaagactctcaggaagcaatccgttttctgaaagccatttcagatgttgcaggtcgctcgattaatcatgacgaatatctgtggccgctgagtatgccgccaaaagtccgtgaagaagatattcaaatcgctcagctggaagatgcgttcgaatatgactaccgcaaatatctggaaaaaacctacggcaaactgattcagtccatctcaggtattcactataacctgggcctgggtcaagaactgctgacctcgctgtttgaactgagccaggcggataacgccattgacttccagaatcaactgtatatgaaactgtctcagaattttctgcgttaccgctggctgctgacctatctgtacggcgccagtccggtggcagaagaagatttcctggaccagaaactgaacaatccggtccgttccctgcgcaactcacatctgggttatgtgaatcacaaagatattcgtatctcgtataccagcctgaaagattacgtgaacgacctggaaaatgctgttaaatctggccagctgattgcggaaaaagaattttatagcccggttcgtctgcgcggctctaaagcctgccgtaactatctggaaaaaggtatcacgtacctggaatttcgcaccttcgatctgaatccgtttagtccgattggtatcacccaggaaacggtggataccgttcacctgtttctgctggcgctgctgtggattgacagctctagtcacatcgatcaggacattaaagaagccaaccgcctgaatgatctgatcgcactgagccatccgctggaaaaactgccgaaccaggctccggtttcagatctggtcgacgcaatgcaatcggtgattcagcactttaatctgagcccgtattaccaagatctgctggaaagtgttaaacgtcagatccaatccccggaactgaccgttgcgggtcagctgctggaaatgattgaaggtctgtccctggaaaccttcggccagcgccagggtcagatctatcatgattacgcatgggaagctccgtatgcgctgaaaggctacgaaacgatggaactgagcacccaactgctgctgttcgatgttattcagaaaggtgtgaactttgaagttctggatgaacaagaccagttcctgaaactgtggcataattcccacatcgaatatgtgaaaaacggcaatatgacgtcaaaagataactacatcgttccgctggcgatggccaataaagtggttaccaagaaaattctggacgaaaaacactttccgacgccgttcggtgatgaatttaccgaccgtaaagaagcgctgaactatttttcgcaaatccaggataaaccgattgtcgtgaaaccgaaaagcacgaacttcggcctgggtatttctatctttaaaaccagtgccaatctggcatcctacgaaaaagctattgatatcgcgtttacggaagacagcgcgatcctggtcgaagaatatattgaaggcaccgaataccgtttctttgtgctggagggtgattgtatcgcggtcctgctgcgtgtggccgcaaatgttgttggtgacggtatccataccatttcccaactggtgaaactgaaaaaccagaatccgctgcgtggctatgatcaccgctcaccgctggaagttatcgaactgggtgaagtcgaacagctgatgctggaacagcaaggctacacggtgaactcgatcccgccggaaggtaccaaaattgaactgcgtcgcaactctaatatcagtacgggcggtgatagcattgacgttaccaatacgatggatccgacctataaacagctggcagctgaaatggcagaagctatgggcgcatgggtctgtggtgtggatctgattatcccgaacgctacgcaggcgtacagcaaagacaagaaaaacgcgacctgcattgaactgaactttaatccgctgatgtatatgcatacctactgtcaagaaggtccgggtcagagcatcacgccgcgcatcctggcaaaactgtttccggaactgtaaaagcttgactcctgttgatagatc

SEQ ID NO: 17 - DNA, 6866 bp, Artificial; identical to SEQ ID NO: 15
  pQEK1-PT5-FSa-term

SEQ ID NO: 18 - DNA, 300 bp, Escherichia coli
gtgactaattttccttgcggaggcttgtctgaagcggtttccgcgattctcttctgtaaattgtcgctgacaaaaaagattaaacataccttatacaagacttttttttcatatgcctgacggagttcacacttgtaagttttcaactacgttgtagactttacatcgccaggggtgctcggcataagccgaagatatcggtagagttaatattgagcagatcccccggtgaaggatttaaccgtgttatctcgttggagatattcatggcgtattttggatgataacgaggcgcaaaaa

SEQ ID NO: 19 - DNA, 300 bp, Escherichia coli
ccagcctgtttacgatgatcccgctgcttaatctgttcatcatgcccgttgccgtttgtggcgcgacggcgatgtgggtcgattgctatcgcgataaacacgcgatgtggcggtaacaatctaccggttattttgtaaaccgtttgtgtgaaacaggggtggcttatgccgccccttattccatcttgcatgtcattatttcccttctgtatatagatatgctaaatccttacttccgcatattctctgagcgggtatgctacctgttgtatcccaatttcatacagttaaggacaggcc

SEQ ID NO: 20 - DNA, 300 bp, Escherichia coli
gtgaattaatttgtatatcgaagcgccctgatgggcgctttttttatttaatcgataaccagaagcaataaaaaatcaaatcggatttcactatataatctcactttatctaagatgaatccgatggaagcatcctgttttctctcaatttttttatctaaaacccagcgttcgatgcttctttgagcgaacgatcaaaaataagtgccttcccatcaaaaaaatattctcaacataaaaaactttgtgtaatacttgtaacgctacatggagattaactcaatctagagggtattaata

SEQ ID NO: 21 - DNA, 822 bp, Escherichia coli
atgtcgtgtgaagaactggaaattgtctggaacaatattaaagccgaagccagaacgctggcggactgtgagccaatgctggccagtttttaccacgcgacgctactcaagcacgaaaaccttggcagtgcactgagctacatgctggcgaacaagctgtcatcgccaattatgcctgctattgctatccgtgaagtggtggaagaagcctacgccgctgacccggaaatgatcgcctctgcggcctgtgatattcaggcggtgcgtacccgcgacccggcagtcgataaatactcaaccccgttgttatacctgaagggttttcatgccttgcaggcctatcgcatcggtcactggttgtggaatcaggggcgtcgcgcactggcaatctttctgcaaaaccaggtttctgtgacgttccaggtcgatattcacccggcagcaaaaattggtcgcggtatcatgcttgaccacgcgacaggcatcgtcgttggtgaaacggcggtgattgaaaacgacgtatcgattctgcaatctgtgacgcttggcggtacgggtaaatctggtggtgaccgtcacccgaaaattcgtgaaggtgtgatgattggcgcgggcgcgaaaatcctcggcaatattgaagttgggcgcggcgcgaagattggcgcaggttccgtggtgctgcaaccggtgccgccgcataccaccgccgctggcgttccggctcgtattgtcggtaaaccagacagcgataagccatcaatggatatggaccagcatttcaacggtattaaccatacatttgagtatggggatgggatctaa

SEQ ID NO: 22 - protein, 273 aa, Escherichia coli
MSCEELEIVWNNIKAEARTLADCEPMLASFYHATLLKHENLGSALSYMLANKLSSPIMPAIAIREVVEEAYAADPEMIASAACDIQAVRTRDPAVDKYSTPLLYLKGFHALQAYRIGHWLWNQGRRALAIFLQNQVSVTFQVDIHPAAKIGRGIMLDHATGIVVGETAVIENDVSILQSVTLGGTGKSGGDRHPKIREGVMIGAGAKILGNIEVGRGAKIGAGSVVLQPVPPHTTAAGVPARIVGKPDSDKPSMDMDQHFNGINHTFEYGDGI

SEQ ID NO: 23 - DNA, 1743 bp, Escherichia coli
atgataaaaccgacgtttttacgccgggtggccattgctgctctgctctcaggaagttgttttagcgccgccgccgcgcctcctgcgccgcccgtctcgtatggtgtggaggaagatgtcttccacccggtacgcgcgaaacagggaatggtagcgtctgtggacgccactgccactcaggtgggggtggatattctcaaggagggcgggaatgccgttgatgccgccgtggcggtgggctacgcgctggcggtaacgcatccgcaggcagggaatctgggcggtggtggttttatgttaatccgctcgaaaaatggcaataccacggctatcgatttccgcgaaatggcacccgccaaagcgacccgcgatatgttcctcgatgatcagggcaacccggacagcaaaaaatcactcacttcgcatctggcttccggcacaccgggtacggtagcaggtttctcgctggcgctggataaatacggcaccatgccgctgaacaaagtcgtgcagcccgcgtttaaactggcacgcgatggttttatcgttaacgacgcgctggctgacgatctcaaaacctacggtagcgaagtgttgccgaatcacgaaaacagtaaagctatcttctggaaagagggcgagccgctgaaaaagggcgacacgctggtgcaggcgaacctggcaaagagcctggagatgattgctgaaaacggcccggacgaattctataaaggcacgattgcggaacagatcgcccaggagatgcagaaaaacggtggcttgatcactaaagaagatttagcagcctataaagcggtcgaacgcactccgataagcggcgattatcgcgggtatcaggtttactccatgccaccgccatcctccggcgggatccatatcgtacaaatcctcaatattctggaaaacttcgatatgaagaaatacggctttggcagcgccgatgcgatgcaaatcatggcagaagcggagaaatacgcctacgccgaccgctcggaatatcttggcgacccggattttgtcaaagtaccgtggcaggcgctgaccaataaagcctatgccaaatctattgccgatcaaattgatatcaataaagcgaagccatccagcgaaattcgccccggcaagcttgcgccttatgagagtaatcaaactacccattactcagtggtggataaagatggtaacgcggtggcggtgacctatacgctgaacaccaccttcggtacgggcattgtcgcgggcgagagcggtattctgcttaataaccagatggatgatttctccgccaaaccgggcgtaccgaacgtttacgggctggtgggcggtgatgccaacgccgtcgggccgaacaaacgcccgctgtcgtcgatgtcgccgaccattgtggtgaaagacggtaaaacctggctggttaccggtagcccaggcggtagccggatcatcactacagtgctgcaaatggtggtgaatagcatcgattatggcttgaacgtcgccgaagcgaccaatgcgccgcgtttccaccatcagtggttgccggacgagctgcgtgtcgaaaaagggtttagcccggatacgctcaagctgctggaagcaaaaggtcagaaagtggcgctgaaagaggcgatgggcagtacacaaagcattatggttgggccggacggtgagttgtacggcgcatccgacccgcgctcggtggatgatttaacggcggggtactaa

SEQ ID NO: 24 - protein, 580 aa, Escherichia coli [sequence omitted]

SEQ ID NO: 25 - DNA, 1227 bp, Escherichia coli
atggataaactacttgagcgatttttgaactacgtgtctctggatacccaatcaaaagcaggggtgagacaggttcccagcacggaaggccaatggaagttattgcatctgctgaaagagcagctcgaagagatggggcttatcaatgtgaccttaagtgagaagggcactttgatggcgacgttaccggctaacgtccctggcgatatcccggcgattggctttatttctcatgtggatacctcaccggattgcagcggcaaaaatgtgaatccgcaaattgttgaaaactatcgcggtggcgatattgcgctgggtatcggcgatgaagttttatcaccggttatgttcccggtgctgcatcagctactgggtcagacgctgattaccaccgatggtaaaaccttgttaggtgccgatgacaaagcaggtattgcagaaatcatgaccgcgctggcggtattgcaacagaaaaaaattccgcatggtgatattcgcgtcgcctttaccccggatgaagaagtgggcaaaggggcgaaacattttgatgttgacgccttcgatgcccgctgggcttacactgttgatggtggtggcgtaggcgaactggagtttgaaaacttcaacgccgcgtcggtcaatatcaaaattgtcggtaacaatgttcatccgggcacggcgaaaggagtgatggtaaatgcgctgtcgctggcggcacgtattcatgcggaagttccggcggatgaaagcccggaaatgacagaaggctatgaaggtttctatcatctggcgagcatgaaaggcaccgttgaacgggccgatatgcactacatcatccgtgatttcgaccgtaaacagtttgaagcgcgtaaacgtaaaatgatggagatcgccaaaaaagtgggcaaagggttacatcctgattgctacattgaactggtgattgaagacagttactacaatatgcgcgagaaagtggttgagcatccgcatattctcgatatcgcccagcaggcgatgcgcgattgcgatattgaaccggaactgaaaccgatccgcggtggtaccgacggcgcgcagttgtcgtttatgggattaccgtgcccgaacctgttcactggcggttacaactatcatggtaagcatgagtttgtgactctggaaggtatggaaaaagcggtgcaggtgatcgtccgtattgccgagttaacggcgcaacggaagtaa

SEQ ID NO: 26 - protein, 408 aa, Escherichia coli
MDKLLERFLNYVSLDTQSKAGVRQVPSTEGQWKLLHLLKEQLEEMGLINVTLSEKGTLMATLPANVPGDIPAIGFISHVDTSPDCSGKNVNPQIVENYRGGDIALGIGDEVLSPVMFPVLHQLLGQTLITTDGKTLLGADDKAGIAEIMTALAVLQQKKIPHGDIRVAFTPDEEVGKGAKHFDVDAFDARWAYTVDGGGVGELEFENFNAASVNIKIVGNNVHPGTAKGVMVNALSLAARIHAEVPADESPEMTEGYEGFYHLASMKGTVERADMHYIIRDFDRKQFEARKRKMMEIAKKVGKGLHPDCYIELVIEDSYYNMREKVVEHPHILDIAQQAMRDCDIEPELKPIRGGTDGAQLSFMGLPCPNLFTGGYNYHGKHEFVTLEGMEKAVQVIVRIAELTAQRK

SEQ ID NO: 27 - DNA, 1353 bp, Escherichia coli
atgactaaacactatgattacatcgccatcggcggcggcagcggcggtatcgcctccatcaaccgcgcggctatgtacggccagaaatgtgcgctgattgaagccaaagagctgggcggcacctgcgtaaatgttggctgtgtgccgaaaaaagtgatgtggcacgcggcgcaaatccgtgaagcgatccatatgtacggcccggattatggttttgataccactatcaataaattcaactgggaaacgttgatcgccagccgtaccgcctatatcgaccgtattcatacttcctatgaaaacgtgctcggtaaaaataacgttgatgtaatcaaaggctttgcccgcttcgttgatgccaaaacgctggaggtaaacggcgaaaccatcacggccgatcatattctgatcgccacaggcggtcgtccgagccacccggatattccgggcgtggaatacggtattgattctgatggcttcttcgcccttcctgctttgccagagcgcgtggcggttgttggcgcgggttacatcgccgttgagctggcgggcgtgattaacggcctcggcgcgaaaacgcatctgtttgtgcgtaaacatgcgccgctgcgcagcttcgacccgatgatttccgaaacgctggtcgaagtgatgaacgccgaaggcccgcagctgcacaccaacgccatcccgaaagcggtagtgaaaaataccgatggtagcctgacgctggagctggaagatggtcgcagtgaaacggtggattgcctgatttgggcgattggtcgcgagcctgccaatgacaacatcaacctggaagccgctggcgttaaaactaacgaaaaaggctatatcgtcgtcgataaatatcaaaacaccaatattgaaggtatttacgcggtgggcgataacacgggtgcagtggagctgacaccggtggcagttgcagcgggtcgccgtctctctgaacgcctgtttaataacaagccggatgagcatctggattacagcaacattccgaccgtggtcttcagccatccgccgattggtactgttggtttaacggaaccgcaggcgcgcgagcagtatggcgacgatcaggtgaaagtgtataaatcctctttcaccgcgatgtataccgccgtcaccactcaccgccagccgtgccgcatgaagctggtgtgcgttggatcggaagagaagattgtcggtattcacggcattggctttggtatggacgaaatgttgcagggcttcgcggtggcgctgaagatgggggcaaccaaaaaagacttcgacaataccgtcgccattcacccaacggcggcagaagagttcgtgacaatgcgttaa

SEQ ID NO: 28 - protein, 450 aa, Escherichia coli
MTKHYDYIAIGGGSGGIASINRAAMYGQKCALIEAKELGGTCVNVGCVPKKVMWHAAQIREAIHMYGPDYGFDTTINKFNWETLIASRTAYIDRIHTSYENVLGKNNVDVIKGFARFVDAKTLEVNGETITADHILIATGGRPSHPDIPGVEYGIDSDGFFALPALPERVAVVGAGYIAVELAGVINGLGAKTHLFVRKHAPLRSFDPMISETLVEVMNAEGPQLHTNAIPKAVVKNTDGSLTLELEDGRSETVDCLIWAIGREPANDNINLEAAGVKTNEKGYIVVDKYQNTNIEGIYAVGDNTGAVELTPVAVAAGRRLSERLFNNKPDEHLDYSNIPTVVFSHPPIGTVGLTEPQAREQYGDDQVKVYKSSFTAMYTAVTTHRQPCRMKLVCVGSEEKIVGIHGIGFGMDEMLQGFAVALKMGATKKDFDNTVAIHPTAAEEFVTMR

SEQ ID NO: 29 - DNA, 1839 bp, Escherichia coli
gtgctggcggttgaaaatctgaatattgcctttatgcaggaccagcagaaaatagctgcggtccgcaatctctcttttagtctgcaacgcggtgagacgctggcaattgttggcgaatccggctccggtaagtcagtgactgcgttggcattgatgcgcctgttggaacaggcgggcggtttagtacagtgcgataaaatgctgttgcagcggcgcagtcgcgaagtgattgaacttagcgagcagaacgctgcacaaatgcgccatgttcgcggtgcggatatggcgatgatatttcaggagccgatgacatcgctgaacccggtatttactgtgggtgaacagattgccgaatcaattcgtctgcatcagaacgccagtcgtgaagaagcgatggtcgaggcgaagcggatgctggatcaggtacgcattcctgaggcacaaaccattctttcacgttatccgcatcaactctctggcgggatgcgccagcgagtgatgattgcgatggcgctgtcatgccgcccggcggtgctgattgccgatgagccaaccaccgcgctggatgtcactattcaggcgcagatcctgcaattaatcaaagtattgcaaaaagagatgtcgatgggcgttatctttatcactcacgatatgggcgtggtggcagagattgccgatcgggtactggtgatgtatcagggcgaggcggtggaaacgggtaccgtcgaacagatttttcatgcaccgcaacatccttacacccgtgcgctgttagctgctgttccgcaacttggtgcgatgaaagggttagattatccccgacgtttcccgttgatatcgcttgaacatccagcgaaacaggccccccccatcgagcagaaaacggtggtggatggcgaacctgttttacgagtgcgtaatcttgtcacccgtttccctttgcgcagcggtttgttgaatcgcgtaacgcgggaagtgcatgccgttgagaaagtcagttttgatctctggcctggcgaaacgctatcgctggtgggcgagtctggcagcggtaaatccactaccgggcgggcgttgctgcgcctggtcgaatcgcagggcggcgaaattatctttaacggtcagcgaatcgataccttgtcacccggcaaacttcaggcattacgccgggatattcagtttatttttcaggacccttacgcttcgctggacccacgtcagaccatcggtgattcgattatcgaaccgctgcgtgtacacggtttattgccaggtaaagacgcggctgcacgcgttgcgtggttgctggagcgcgtgggcctgttacctgaacatgcctggcgttacccgcatgagttttccggcggtcagcgccagcgcatctgcattgctcgcgcgttggcattgaatccaaaagtgatcattgccgacgaagccgtttcggcgctggatgtttctattcgcgggcagattatcaacttgttgctcgatctccagcgtgatttcggcattgcgtatctgtttatctcccacgatatggcggtggtagagcggattagtcatcgtgtggcggtgatgtatctcgggcaaattgttgaaattggtccacggcgcgcggtcttcgaaaacccgcagcatccttatacgcgtaaattactggcggcagttccggtcgctgaaccgtcccgacaacgaccgcagcgtgtactgctgtcggacgatcttcccagcaatattcatctgcgtggcgaagaggtggcagccgtctcgttgcaatgcgtcgggccggggcattacgtcgcacaaccacaatcagaatacgcattcatgcgtagataa

SEQ ID NO: 30 - protein, 612 aa, Escherichia coli
MLAVENLNIAFMQDQQKIAAVRNLSFSLQRGETLAIVGESGSGKSVTALALMRLLEQAGGLVQCDKMLLQRRSREVIELSEQNAAQMRHVRGADMAMIFQEPMTSLNPVFTVGEQIAESIRLHQNASREEAMVEAKRMLDQVRIPEAQTILSRYPHQLSGGMRQRVMIAMALSCRPAVLIADEPTTALDVTIQAQILQLIKVLQKEMSMGVIFITHDMGVVAEIADRVLVMYQGEAVETGTVEQIFHAPQHPYTRALLAAVPQLGAMKGLDYPRRFPLISLEHPAKQAPPIEQKTVVDGEPVLRVRNLVTRFPLRSGLLNRVTREVHAVEKVSFDLWPGETLSLVGESGSGKSTTGRALLRLVESQGGEIIFNGQRIDTLSPGKLQALRRDIQFIFQDPYASLDPRQTIGDSIIEPLRVHGLLPGKDAAARVAWLLERVGLLPEHAWRYPHEFSGGQRQRICIARALALNPKVIIADEAVSALDVSIRGQIINLLLDLQRDFGIAYLFISHDMAVVERISHRVAVMYLGQIVEIGPRRAVFENPQHPYTRKLLAAVPVAEPSRQRPQRVLLSDDLPSNIHLRGEEVAAVSLQCVGPGHYVAQPQSEYAFMRR

SEQ ID NO: 31 - DNA, 1539 bp, Escherichia coli
atggcaagagctgtacaccgtagtgggttagtggcgctgggcattgcgacagcgttgatggcatcttgtgcattcgctgccaaagatgtggtggtggcggtaggatcgaatttcaccacgctcgatccgtatgacgcaaatgacacgttatctcaggccgtagcgaaatcgttttaccaggggctgttcggtctggataaagagatgaaactgaaaaacgtgctggcggagagttataccgtttccgatgacggcattacttacaccgtgaaattgcgggaaggcattaaattccaggatggcaccgatttcaacgccgcggcggtgaaagcgaatctggaccgggccagcgatccggcgaatcatcttaaacgctataacctgtataagaatattgctaaaacggaagcgatcgatccgacaacggtaaagattaccctcaaacagccgttctcagcgtttattaatattcttgcccatccggcgaccgcgatgatttcaccggcagcgctggaaaaatatggcaaggagattggtttttatccggtgggaaccggaccgtatgaactggatacctggaatcagaccgattttgtgaaggtgaaaaaattcgcgggttactggcagccaggattgcccaaactggacagcataacctggcgtccggtggcggataacaacacccgcgcggcaatgctgcaaaccggtgaagcgcagtttgctttccccattccttacgagcaggccacactgctggagaaaaacaaaaatatcgagttgatggccagtccgtcaattatgcagcgttatatcagtatgaacgtgacgcaaaagccgttcgataacccgaaggtccgtgaggcgctgaattacgccattaaccgtccggcgctggtgaaagttgcctttgcgggctatgcaacgccagctactggtgtggtaccgccaagtatcgcctacgcgcaaagttataaaccgtggccttacgatccagtgaaagcgcgcgaattactgaaagaggcgggatatcccaacggtttcagtaccacgctgtggtcgtcacataaccacagcaccgcgcagaaagtgctgcaatttacccagcagcagttagcgcaggtcgggattaaagcccaggtgactgcgatggatgccggacagcgggcggcagaagttgaaggtaaagggcaaaaagagagcggcgtgcggatgttctacactggctggtcggcttcaaccggcgaagcggactgggcactatcgccgctgtttgcctcgcagaactggccaccgacgctgtttaataccgcgttttacagcaataaacaggtggatgacttcctggctcaggcactgaaaactaatgatccggcggaaaagacccgcttatataaggcggcgcaggatatcatctggcaagaatcgccgtggatcccgctggtggtagaaaaactggtgtcggcacacagtaaaaacctgaccggtttttggatcatgccagacaccggcttcagctttgaagacgcggatttgcaataa

SEQ ID NO: 32 - protein, 512 aa, Escherichia coli
MARAVHRSGLVALGIATALMASCAFAAKDVVVAVGSNFTTLDPYDANDTLSQAVAKSFYQGLFGLDKEMKLKNVLAESYTVSDDGITYTVKLREGIKFQDGTDFNAAAVKANLDRASDPANHLKRYNLYKNIAKTEAIDPTTVKITLKQPFSAFINILAHPATAMISPAALEKYGKEIGFYPVGTGPYELDTWNQTDFVKVKKFAGYWQPGLPKLDSITWRPVADNNTRAAMLQTGEAQFAFPIPYEQATLLEKNKNIELMASPSIMQRYISMNVTQKPFDNPKVREALNYAINRPALVKVAFAGYATPATGVVPPSIAYAQSYKPWPYDPVKARELLKEAGYPNGFSTTLWSSHNHSTAQKVLQFTQQQLAQVGIKAQVTAMDAGQRAAEVEGKGQKESGVRMFYTGWSASTGEADWALSPLFASQNWPPTLFNTAFYSNKQVDDFLAQALKTNDPAEKTRLYKAAQDIIWQESPWIPLVVEKLVSAHSKNLTGFWIMPDTGFSFEDADLQ

SEQ ID NO: 33 - DNA, 921 bp, Escherichia coli
atgcttaattacgttatcaaacgcttactggggttgattccgacgctgtttatcgtctcggtgctggtgtttttatttgtccatatgctgcccggcgatccggcgcgattgattgccgggcccgaagctgatgcgcaggttatagaactggtgcgtcagcagctggggttggatcagccgctgtatcaccagttctggcactatatcagcaatgctgtgcagggggattttggcctgtcgatggtgtcgcgtcgtccggttgccgatgagattgccagccgctttatgccaacgctgtggctgaccataaccagtatggtctgggcggttatatttggtatggcggcgggaattatcgccgccgtctggcgtaaccgttggccggatcgattgagtatgaccattgcggtgtcggggatctcgtttccggcatttgctctggggatgcttttaattcaggtattctccgttgaactgggctggctgcctaccgtgggagcagacagttggcagcactacattttaccctccctgacgctcggcgcggcagtggccgccgtgatggcgcgctttacccgcgcgtcgtttgtcgatgttttaagcgaagattatatgcgtaccgcgagggcgaaaggggtgagcgaaacctgggttgtcctcaaacacgggctacgtaacgcgatgatcccggtagtgaccatgatgggcttacagtttggctttttgctcggtggttccatcgttgtggagaaagttttcaactggccgggacttggacgcttactcgttgactccgtagaaatgcgtgattacccggtgattcaggcggaaattctgcttttctcgctggaatttattcttatcaacttagtggtggatgtgctttacgccgccattaacccggctatcaggtacaagtaa

SEQ ID NO: 34 - protein, 306 aa, Escherichia coli
MLNYVIKRLLGLIPTLFIVSVLVFLFVHMLPGDPARLIAGPEADAQVIELVRQQLGLDQPLYHQFWHYISNAVQGDFGLSMVSRRPVADEIASRFMPTLWLTITSMVWAVIFGMAAGIIAAVWRNRWPDRLSMTIAVSGISFPAFALGMLLIQVFSVELGWLPTVGADSWQHYILPSLTLGAAVAAVMARFTRASFVDVLSEDYMRTARAKGVSETWVVLKHGLRNAMIPVVTMMGLQFGFLLGGSIVVEKVFNWPGLGRLLVDSVEMRDYPVIQAEILLFSLEFILINLVVDVLYAAINPAIRYK

SEQ ID NO: 35 - DNA, 912 bp, Escherichia coli
atgcgactatttaactggcgacgtcaggcggtgttaaacgccatgccactggtcaaacctgaccaggtacgtacaccgtggcatgaattctggcgacgatttcgccgtcagcatatggcgatgaccgccgcattattcgttattttattgattgtggtggccatttttgcacgctggatcgctccctatgacgccgaaaattattttgattatgacaatctgaataacggaccttctttgcagcactggtttggcgtcgattcactggggcgtgacattttcagccgtgtcctggttggtgcgcaaatctcgctggcggcgggcgtgtttgccgtgtttatcggtgcggcgatcgggacgttgctgggcttgctcgctggatattatgaaggctggtgggatcggctgatcatgcgcatttgcgatgtgctgtttgccttcccgggtattttactggcgatcgctgttgttgcggtgttgggaagcggcattgctaacgtgattattgcagtcgccattttttccatccccgcgtttgcccgcctggtgcgcggcaacacgctggtgttgaaacagcaaacctttattgagtcagcacgcagtattggtgccagcgatatgaccgttttgttgcgtcatatcctgcctgggaccgtctcttctatcgtggtgtttttcaccatgcgcattggtacctcgattatctctgccgccagcctctcatttctcggcctcggtgcgcagccgccgacaccagagtggggagcaatgctcaatgaggctcgagcggatatggttatcgcgccgcatgtcgctgtttttccggccctggctatttttctgaccgtactggcgttcaatttgttgggcgatggtttacgcgatgcgctggatccgaaaattaaaggatag

SEQ ID NO: 36 - protein, 303 aa, Escherichia coli
MRLFNWRRQAVLNAMPLVKPDQVRTPWHEFWRRFRRQHMAMTAALFVILLIVVAIFARWIAPYDAENYFDYDNLNNGPSLQHWFGVDSLGRDIFSRVLVGAQISLAAGVFAVFIGAAIGTLLGLLAGYYEGWWDRLIMRICDVLFAFPGILLAIAVVAVLGSGIANVIIAVAIFSIPAFARLVRGNTLVLKQQTFIESARSIGASDMTVLLRHILPGTVSSIVVFFTMRIGTSIISAASLSFLGLGAQPPTPEWGAMLNEARADMVIAPHVAVFPALAIFLTVLAFNLLGDGLRDALDPKIKG

SEQ ID NO: 37 - DNA, 1416 bp, Escherichia coli
atggaaaactttaaacatctccctgaaccgttccgcattcgtgttattgagccagtaaaacgtaccactcgcgcttatcgtgaagaggcaattattaaatccggtatgaacccgttcctgctggatagcgaagatgtttttatcgatttactgaccgacagcggcaccggggcggtgacgcagagcatgcaggctgcgatgatgcgcggcgacgaagcctacagcggcagtcgtagctactatgcgttagccgagtcagtgaaaaatatctttggttatcaatacaccattccgactcaccagggccgtggcgcagagcaaatctatattccggtactgattaaaaaacgcgagcaggaaaaaggcctggatcgcagcaaaatggtggcgttctctaactatttctttgataccacgcagggccatagccagatcaacggctgtaccgtgcgtaacgtctatatcaaagaagccttcgatacgggcgtgcgttacgactttaaaggcaactttgaccttgagggattagaacgcggtattgaagaagttggtccgaataacgtgccgtatatcgttgcaaccatcaccagtaactctgcaggtggtcagccggtttcactggcaaacttaaaagcgatgtacagcatcgcgaagaaatacgatattccggtggtaatggactccgcgcgctttgctgaaaacgcctatttcatcaagcagcgtgaagcagaatacaaagactggaccatcgagcagatcacccgcgaaacctacaaatatgccgatatgctggcgatgtccgccaagaaagatgcgatggtgccgatgggcggcctgctgtgcatgaaagacgacagcttctttgatgtgtacaccgagtgcagaaccctttgcgtggtgcaggaaggcttcccgacatatggcggcctggaaggcggcgcgatggagcgtctggcggtaggtctgtatgacggcatgaatctcgactggctggcttatcgtatcgcgcaggtacagtatctggtcgatggtctggaagagattggcgttgtctgccagcaggcgggcggtcacgcggcattcgttgatgccggtaaactgttgccgcatatcccggcagaccagttcccggcacaggcgctggcctgcgagctgtataaagtcgccggtatccgtgcggtagaaattggctctttcctgttaggccgcgatccgaaaaccggtaaacaactgccatgcccggctgaactgctgcgtttaaccattccgcgcgcaacatatactcaaacacatatggacttcattattgaagcctttaaacatgtgaaagagaacgcggcgaatattaaaggattaacctttacgtacgaaccgaaagtattgcgtcacttcaccgcaaaacttaaagaagtttaa

SEQ ID NO: 38 - protein, 471 aa, Escherichia coli
MENFKHLPEPFRIRVIEPVKRTTRAYREEAIIKSGMNPFLLDSEDVFIDLLTDSGTGAVTQSMQAAMMRGDEAYSGSRSYYALAESVKNIFGYQYTIPTHQGRGAEQIYIPVLIKKREQEKGLDRSKMVAFSNYFFDTTQGHSQINGCTVRNVYIKEAFDTGVRYDFKGNFDLEGLERGIEEVGPNNVPYIVATITSNSAGGQPVSLANLKAMYSIAKKYDIPVVMDSARFAENAYFIKQREAEYKDWTIEQITRETYKYADMLAMSAKKDAMVPMGGLLCMKDDSFFDVYTECRTLCVVQEGFPTYGGLEGGAMERLAVGLYDGMNLDWLAYRIAQVQYLVDGLEEIGVVCQQAGGHAAFVDAGKLLPHIPADQFPAQALACELYKVAGIRAVEIGSFLLGRDPKTGKQLPCPAELLRLTIPRATYTQTHMDFIIEAFKHVKENAANIKGLTFTYEPKVLRHFTAKLKEV

SEQ ID NO: 39 - DNA, 1644 bp, Escherichia coli
atgcgccaggtattatcgtctcttttggtgattgctggacttgtgagtggtcaggcaatcgccgcgcctgaatctcccccgcatgctgatatccgcgacagcggttttgtctattgcgtcagcgggcaagtcaacacctttaacccatccaaagcgagcagtgggttaattgtcgatacccttgccgcccagttttatgatcgactgctggatgtcgatccctatacctatcgcctgatgccggaacttgccgaaagctgggaagtactcgacaacggcgcgacctatcgcttccacctgcgtcgcgatgttccgtttcaaaaaaccgactggtttactcccactcgtaaaatgaatgccgacgatgtggtgtttaccttccagcgaatttttgaccgcaacaacccgtggcataacgtcaacggcagcaacttcccctacttcgacagcctgcaatttgccgataacgtgaaaagcgtccgcaaactggataatcataccgttgagttccgactcgcccagccggatgcttcttttttgtggcacctcgcaacccattatgcttcggtcatgtcggcagaatatgcccggaagttagagaaagaagatcgccaggagcaactcgaccgtcaaccggtcggcaccggtccgtatcagttgtcggaataccgcgccgggcaatttattcgcctacaacgtcatgatgacttctggcgcggtaaaccgttaatgccgcaggtagtggtggatttaggctccggcggcaccggacgtctgtcgaaactcctgaccggggaatgcgacgttctggcctggcctgctgccagccagctatccattttgcgtgacgacccgcgcttgcgtttaacgctgcgtcctgggatgaacgtcgcctatctggcatttaacaccgccaaaccgccgctaaataatcccgctgtccgccatgcgctggcactggcgattaataaccagcgcctgatgcaatccatctattatggtacggctgaaacggcggcctctattttaccgcgcgcctcgtgggcctatgacaacgaggctaaaattactgaatacaatccggcgaaatcgcgcgaacagttgaagtcgttggggctggaaaatttaacgctgaaactgtgggtgcccacacgttcgcaggcgtggaaccccagtccactgaaaactgccgaactgattcaggcggatatggcgcaggttggcgtaaaagtggtgattgtgccggtagaaggtcgctttcaggaggcgcggttgatggatatgagccatgatctgacgttatccggttgggcgacggacagtaacgacccggacagtttcttccgtccgttactgagctgcgcggcaattcattcgcagaccaacctcgcccactggtgcgatccgaaattcgacagcgtattgcgtaaggcgctctcttcgcagcagctggcggcgcgtattgaagcctatgacgaagcgcagagtattctggcgcaggaattgcccattttgccgctggcgtcgtcattgcgtttgcaggcctatcggtacgatatcaaaggtctggtacttagcccgtttggtaacgcctcctttgctggggtgtatcgcgagaaacaggatgaggtgaaaaaaccatga

SEQ ID NO: 40 - protein, 547 aa, Escherichia coli
MRQVLSSLLVIAGLVSGQAIAAPESPPHADIRDSGFVYCVSGQVNTFNPSKASSGLIVDTLAAQFYDRLLDVDPYTYRLMPELAESWEVLDNGATYRFHLRRDVPFQKTDWFTPTRKMNADDVVFTFQRIFDRNNPWHNVNGSNFPYFDSLQFADNVKSVRKLDNHTVEFRLAQPDASFLWHLATHYASVMSAEYARKLEKEDRQEQLDRQPVGTGPYQLSEYRAGQFIRLQRHDDFWRGKPLMPQVVVDLGSGGTGRLSKLLTGECDVLAWPAASQLSILRDDPRLRLTLRPGMNVAYLAFNTAKPPLNNPAVRHALALAINNQRLMQSIYYGTAETAASILPRASWAYDNEAKITEYNPAKSREQLKSLGLENLTLKLWVPTRSQAWNPSPLKTAELIQADMAQVGVKVVIVPVEGRFQEARLMDMSHDLTLSGWATDSNDPDSFFRPLLSCAAIHSQTNLAHWCDPKFDSVLRKALSSQQLAARIEAYDEAQSILAQELPILPLASSLRLQAYRYDIKGLVLSPFGNASFAGVYREKQDEVKKP

SEQ ID NO: 41 - DNA, 966 bp, Escherichia coli
atgattatcttcaccttacgccgcattttgttattgattgtcaccttgttcctgctgaccttcgttggcttcagcttgagctatttcacccctcatgcgccgttacaaggcgcgtcactgtggaatgcctgggtgttctggtttaacggcctgatccactgggattttggtgtttccagcattaacggtcaaccgattgccgaacagttaaaagaggtcttcccggcgacgatggaactgtgcatcctcgccttcggttttgcgctgattgttggcatccccgtggggatgattgccgggattacgcgccataagtggcaggataacttgatcaacgccatcgccttactgggcttttcaatcccggtgttctggctggcgcttctgttgacgctgttttgttcactcacgctaggttggttgcccgtttcagggcgtttcgatctgctctacgaagtgaaaccgattaccggttttgcgttgattgatgcctggctttcggactcaccatggcgggatgaaatgatcatgagcgcaatccgccatatgatattgcccgtgatcaccctgtcggttgcgccaacaactgaagtgatccgcctgatgcgtatcagcactatcgaagtgtatgaccagaactatgtaaaagcggcggcgacccgcggtttgtcgcgctttactattttgcgtcgccacgttctgcataacgcgttaccgccggttattcctcgcctgggcttacagttttccaccatgctgacgctggcgatgatcaccgaaatggtttttagctggccgggtttagggcgctggttaattaacgccatccgccagcaggattacgcagccatttccgccggagtgatggtgtgtggctcactggtcattattgttaacgtgatttctgatattttgggtgccatggctaaccctctgaaacataaggaatggtatgccttacgatag

SEQ ID NO: 42 - protein, 321 aa, Escherichia coli
MIIFTLRRILLLIVTLFLLTFVGFSLSYFTPHAPLQGASLWNAWVFWFNGLIHWDFGVSSINGQPIAEQLKEVFPATMELCILAFGFALIVGIPVGMIAGITRHKWQDNLINAIALLGFSIPVFWLALLLTLFCSLTLGWLPVSGRFDLLYEVKPITGFALIDAWLSDSPWRDEMIMSAIRHMILPVITLSVAPTTEVIRLMRISTIEVYDQNYVKAAATRGLSRFTILRRHVLHNALPPVIPRLGLQFSTMLTLAMITEMVFSWPGLGRWLINAIRQQDYAAISAGVMVCGSLVIIVNVISDILGAMANPLKHKEWYALR

SEQ ID NO: 43 - DNA, 891 bp, Escherichia coli
atgccttacgatagcgtatacagcgaaaagcgcccgccgggcacgctgcgtaccgcctggcgcaaattttatagtgacgcctctgcgatggtcggcctgtacggctgcgcggggctggctgtactgtgtatttttggcggctggtttgcgccttacggcatcgaccagcaatttctcggttatcaattattgccgccgtcatggtcgcgctatggcgaagtttctttcttcctggggactgacgacctcggacgcgatgttttaagccgtttactgagcggagctgcgccaaccgtgggtggcgcatttgtggtgacgcttgccgcgacgatctgtggcctggtactgggcaccttcgccggggcgacacacggcctccgctcagcggtgcttaaccatattctggataccttactggcgatcccttcgctgctgttggcaattatcgttgtagcgtttgccggaccgagtttgtcccacgctatgtttgctgtctggctggcattgctgccgcgtatggtacgttcgatttacagcatggtgcatgacgaactggaaaaagagtacgttatcgccgcccgtctggatggcgcatcaacgctgaatattctctggtttgctgtgatgccaaacatcaccgccgggctggtcaccgagatcacccgcgcactgtcgatggcaattctcgatatcgccgcgctgggctttctcgatctcggcgcacaactcccctcgcctgaatggggagcgatgctcggtgatgcgctggaactgatttatgtcgccccgtggactgtcatgctgccaggtgcggcaattatgattagcgtgttgttggttaacctccttggcgatggcgttcgtcgtgcgattattgcgggggtggaataa

SEQ ID NO: 44 - protein, 296 aa, Escherichia coli
MPYDSVYSEKRPPGTLRTAWRKFYSDASAMVGLYGCAGLAVLCIFGGWFAPYGIDQQFLGYQLLPPSWSRYGEVSFFLGTDDLGRDVLSRLLSGAAPTVGGAFVVTLAATICGLVLGTFAGATHGLRSAVLNHILDTLLAIPSLLLAIIVVAFAGPSLSHAMFAVWLALLPRMVRSIYSMVHDELEKEYVIAARLDGASTLNILWFAVMPNITAGLVTEITRALSMAILDIAALGFLDLGAQLPSPEWGAMLGDALELIYVAPWTVMLPGAAIMISVLLVNLLGDGVRRAIIAGVE

SEQ ID NO: 45 - DNA, 993 bp, Escherichia coli
atgccattactcgatattcgtaacctgaccattgaatttaaaaccggtgatgagtgggttaaagccgtcgaccgggtaagtatgaccttaaccgaaggtgaaatccgcggtcttgttggtgaatccggttccggcaagagtttgattgcgaaagcgatttgtggggtgaataaagataactggcgtgttactgctgaccgtatgcgttttgatgatatcgatttgctgcgtctctccgcacgcgaacggcgcaaactggttggccataacgtgtcgatgattttccaggaaccgcagtcgtgtcttgacccttcagaacgtgtgggccgccagttgatgcaaaacatcccagcctggacctataaaggccgttggtggcagcgttttggctggcgcaaacgccgtgccattgaactgctgcaccgcgtggggattaaagatcacaaagatgcgatgcgcagttttccctatgagttgaccgaaggtgaatgtcagaaagtgatgatagccattgcactggcgaatcaaccgcgtctgctgattgctgacgaaccgaccaactcaatggagccaacaacccaggcgcaaatctttcgcctgctgacgcgtctcaaccaaaacagcaataccactattttgcttatcagccatgacttacaaatgcttagccaatgggcggataaaattaacgtgctttactgcggtcaaacagtggaaaccgcgccgagtaaggagttggtgacgatgccacatcatccttatacccaggcgctgatccgcgcgataccagacttcggcagcgcgatgccgcataaaagtcgcctcaatacgctgcccggcgctatcccactgctggaacagttaccgattggttgtcgtctggggccacgttgcccgtatgcacaacgagaatgcattgtgacgccacgtttgacgggggcgaaaaatcatctctatgcctgtcatttcccgctgaacatggagaaagagtga

SEQ ID NO: 46 - protein, 330 aa, Escherichia coli
MPLLDIRNLTIEFKTGDEWVKAVDRVSMTLTEGEIRGLVGESGSGKSLIAKAICGVNKDNWRVTADRMRFDDIDLLRLSARERRKLVGHNVSMIFQEPQSCLDPSERVGRQLMQNIPAWTYKGRWWQRFGWRKRRAIELLHRVGIKDHKDAMRSFPYELTEGECQKVMIAIALANQPRLLIADEPTNSMEPTTQAQIFRLLTRLNQNSNTTILLISHDLQMLSQWADKINVLYCGQTVETAPSKELVTMPHHPYTQALIRAIPDFGSAMPHKSRLNTLPGAIPLLEQLPIGCRLGPRCPYAQRECIVTPRLTGAKNHLYACHFPLNMEKE

SEQ ID NO: 47 - DNA, 807 bp, Escherichia coli
atgatcgaaacgctgcttgaagtgcgtaatttaagtaaaacgttccgctaccggaccggctggtttcgtcgtcagaccgtagaagcggtaaaacccttgagctttacgctacgtgaaggccagacactggcgattattggcgagaatggttcgggtaaatccacgctggcaaaaatgctggcgggaatgatagagcctaccagcggcgagttattgattgacgatcatccactgcattttggcgattattccttccgcagtcagcgtattcgcatgatttttcaggatccttcgacctcgttgaatccccgtcaacgtatctcgcaaattctggattttccactgcgcctgaacaccgacctggaaccggaacagcgtcgcaaacagattattgaaacgatgcgtatggttgggctgctaccggatcacgtcagttactatccgcatatgttggcacccggacaaaaacagcgtctgggtctggcgcgcgcgctgatattgcgcccaaaagtcattattgccgatgaagccctcgcttcactggatatgtcgatgcgttcgcagttgattaatctgatgctggaattacaggaaaaacagggcatttcgtatatttatgttacccagcatatcggaatgatgaagcacattagcgaccaggtgctggtgatgcatcagggcgaggttgtcgagcgaggcagcaccgcggatgtgctggcatcgccgctgcatgaactcaccaaacggctgattgccggtcattttggcgaggcattgacggcggatgcgtggcgtaaagatcgctaa

SEQ ID NO: 48 - protein, 268 aa, Escherichia coli
MIETLLEVRNLSKTFRYRTGWFRRQTVEAVKPLSFTLREGQTLAIIGENGSGKSTLAKMLAGMIEPTSGELLIDDHPLHFGDYSFRSQRIRMIFQDPSTSLNPRQRISQILDFPLRLNTDLEPEQRRKQIIETMRMVGLLPDHVSYYPHMLAPGQKQRLGLARALILRPKVIIADEALASLDMSMRSQLINLMLELQEKQGISYIYVTQHIGMMKHISDQVLVMHQGEVVERGSTADVLASPLHELTKRLIAGHFGEALTADAWRKDR

SEQ ID NO: 49 - DNA, 945 bp, Thiobacillus denitrificans
atgaagctgctgttcgtcgtcgatccgctggcgagcctcaagccgtacaaggacagttcggtcgcgatgatgcgcgcggcctgtgcgcgcggccacgccgtgttcgcggccgaagcacgcgcgctgctggtgcgcgacggagtcgcgcggtcgcgcgccgacgccgtcgagacgcgcggcgacgacgattggtatcgcgtgaccgaaacgcgcgaattcgcgctcacggatttcgatgccgttgtgatgcgcgccgacccgccggtcgacgtcgactacctgctcgcgacccacctgctcggcgtcgccgagaccaacggcgcgcgcgtgctgaaccggccgcgcgcgctgcgcgacttcaacgaaaaactcgccatcctcgagtttccgcaatttgtcgccccgacgctggtttcggccgacgcgacagaaatcgcccacttcctcgccgcccacgccgacatcatcgtcaagccgctcaccgagatgggcggcagcggcgtcttccgcctcggcgtttccgacccgaaccgcaacgccatcctcgaaacgctcacccgacgcggcagccggccgatcatggcgcagcgttatctgccggcgatcagcgaaggcgacaagcgcatcctgctgatcgacggcgaggtggtgccgtgggccctcgcgcggattccgctgacgggcgagacgcgcggcaatctcgccgcgggcggcacggcgcgtgcccagccgctttcggaacgcgaccgcgagatcgccgagacgatcgcgccctgggtgcgctcgcaaggcatcttcctcgccggcctcgacgtgatcggcgactgcctcaccgaaatcaacgtcacgagcccgaccggctttcaggaaatcacggcgcagagcggccacgacgtcgcggaccagttcatcgccgcgatcgagcgtgccacgcgtccggaataa

SEQ ID NO: 50 - protein, 314 aa, Thiobacillus denitrificans
MKLLFVVDPLASLKPYKDSSVAMMRAACARGHAVFAAEARALLVRDGVARSRADAVETRGDDDWYRVTETREFALTDFDAVVMRADPPVDVDYLLATHLLGVAETNGARVLNRPRALRDFNEKLAILEFPQFVAPTLVSADATEIAHFLAAHADIIVKPLTEMGGSGVFRLGVSDPNRNAILETLTRRGSRPIMAQRYLPAISEGDKRILLIDGEVVPWALARIPLTGETRGNLAAGGTARAQPLSERDREIAETIAPWVRSQGIFLAGLDVIGDCLTEINVTSPTGFQEITAQSGHDVADQFIAAIERATRPE

SEQ ID NO: 51 - DNA, 948 bp, Artificial
  TDgshB(V260A)
atgaaactgctgttcgtcgttgatcccctggccagcttgaaaccgtacaaggatagctccgttgccatgatgcgcgcagcgtgtgctcgtggtcatgccgtgttcgcagcagaagcgcgcgcactgctggttcgtgatggggtggctcgttctcgtgcagatgctgtcgaaacgcgtggcgacgatgactggtatcgcgttaccgaaacgcgtgaatttgccttaaccgactttgatgcagtggtgatgcgcgcagatccgcccgttgacgtggattaccttctcgcgacgcacctgttaggcgtagccgaaaccaacggtgcacgtgtcctgaatcgcccgcgtgccttgcgcgatttcaacgagaaactggccattctggaatttccgcagtttgtcgcacctaccctggtaagtgcggacgcaaccgaaattgcccactttctggctgctcatgcggatatcatcgtcaaaccgctgactgagatgggtggctccggtgtgtttcgcctgggagttagcgatccgaatcggaacgcgattctggagacattaacccgtcgtggctctcgcccaatcatggctcagcggtatttgccagcgatctcagagggcgacaaacgcatcctgctgatcgacggcgaagtagtgccatgggccttggcgcgcattccgctgaccggtgaaactcgcgggaatcttgcggctggtggtacagcgcgcgcgcaaccgctcagtgaacgggatcgcgaaatcgccgaaacgattgccccttgggcacgcagccagggcattttccttgcgggcttagacgtgattggggattgcctcaccgagattaacgtgacatcgcctactggatttcaggagattaccgcccaatcgggccatgatgttgcggaccagttcattgcggcgatcgaacgcgcgacgcgtccggaatgataa

SEQ ID NO: 52 - protein, 314 aa, Artificial
  TDgshB(V260A)
MKLLFVVDPLASLKPYKDSSVAMMRAACARGHAVFAAEARALLVRDGVARSRADAVETRGDDDWYRVTETREFALTDFDAVVMRADPPVDVDYLLATHLLGVAETNGARVLNRPRALRDFNEKLAILEFPQFVAPTLVSADATEIAHFLAAHADIIVKPLTEMGGSGVFRLGVSDPNRNAILETLTRRGSRPIMAQRYLPAISEGDKRILLIDGEVVPWALARIPLTGETRGNLAAGGTARAQPLSERDREIAETIAPWARSQGIFLAGLDVIGDCLTEINVTSPTGFQEITAQSGHDVADQFIAAIERATRPE

SEQ ID NO: 53 - DNA, 2253 bp, Streptococcus agalactiae
atgattattgaccgtctgctgcaacgctcccatagccatctgccgatcctgcaagccacctttggtctggaacgtgaatccctgcgcattcatcagccgacccagcgtgtggcccagacgccgcatccgaaaaccctgggctctcgcaactatcacccgtacatccaaacggattatagtgaaccgcagctggaactgattaccccgatcgccaaagactctcaggaagcaatccgttttctgaaagccatttcagatgttgcaggtcgctcgattaatcatgacgaatatctgtggccgctgagtatgccgccaaaagtccgtgaagaagatattcaaatcgctcagctggaagatgcgttcgaatatgactaccgcaaatatctggaaaaaacctacggcaaactgattcagtccatctcaggtattcactataacctgggcctgggtcaagaactgctgacctcgctgtttgaactgagccaggcggataacgccattgacttccagaatcaactgtatatgaaactgtctcagaattttctgcgttaccgctggctgctgacctatctgtacggcgccagtccggtggcagaagaagatttcctggaccagaaactgaacaatccggtccgttccctgcgcaactcacatctgggttatgtgaatcacaaagatattcgtatctcgtataccagcctgaaagattacgtgaacgacctggaaaatgctgttaaatctggccagctgattgcggaaaaagaattttatagcccggttcgtctgcgcggctctaaagcctgccgtaactatctggaaaaaggtatcacgtacctggaatttcgcaccttcgatctgaatccgtttagtccgattggtatcacccaggaaacggtggataccgttcacctgtttctgctggcgctgctgtggattgacagctctagtcacatcgatcaggacattaaagaagccaaccgcctgaatgatctgatcgcactgagccatccgctggaaaaactgccgaaccaggctccggtttcagatctggtcgacgcaatgcaatcggtgattcagcactttaatctgagcccgtattaccaagatctgctggaaagtgttaaacgtcagatccaatccccggaactgaccgttgcgggtcagctgctggaaatgattgaaggtctgtccctggaaaccttcggccagcgccagggtcagatctatcatgattacgcatgggaagctccgtatgcgctgaaaggctacgaaacgatggaactgagcacccaactgctgctgttcgatgttattcagaaaggtgtgaactttgaagttctggatgaacaagaccagttcctgaaactgtggcataattcccacatcgaatatgtgaaaaacggcaatatgacgtcaaaagataactacatcgttccgctggcgatggccaataaagtggttaccaagaaaattctggacgaaaaacactttccgacgccgttcggtgatgaatttaccgaccgtaaagaagcgctgaactatttttcgcaaatccaggataaaccgattgtcgtgaaaccgaaaagcacgaacttcggcctgggtatttctatctttaaaaccagtgccaatctggcatcctacgaaaaagctattgatatcgcgtttacggaagacagcgcgatcctggtcgaagaatatattgaaggcaccgaataccgtttctttgtgctggagggtgattgtatcgcggtcctgctgcgtgtggccgcaaatgttgttggtgacggtatccataccatttcccaactggtgaaactgaaaaaccagaatccgctgcgtggctatgatcaccgctcaccgctggaagttatcgaactgggtgaagtcgaacagctgatgctggaacagcaaggctacacggtgaactcgatcccgccggaaggtaccaaaattgaactgcgtcgcaactctaatatcagtacgggcggtgatagcattgacgttaccaatacgatggatccgacctataaacagctggcagctgaaatggcagaagctatgggcgcatgggtctgtggtgtggatctgattatcccgaacgctacgcaggcgtacagcaaagacaagaaaaacgcgacctgcattgaactgaactttaatccgctgatgtatatgcatacctactgtcaagaaggtccgggtcagagcatcacgccgcgcatcctggcaaaactgtttccggaactgtaa

SEQ ID NO: 54 - protein, 750 aa, Streptococcus agalactiae
MIIDRLLQRSHSHLPILQATFGLERESLRIHQPTQRVAQTPHPKTLGSRNYHPYIQTDYSEPQLELITPIAKDSQEAIRFLKAISDVAGRSINHDEYLWPLSMPPKVREEDIQIAQLEDAFEYDYRKYLEKTYGKLIQSISGIHYNLGLGQELLTSLFELSQADNAIDFQNQLYMKLSQNFLRYRWLLTYLYGASPVAEEDFLDQKLNNPVRSLRNSHLGYVNHKDIRISYTSLKDYVNDLENAVKSGQLIAEKEFYSPVRLRGSKACRNYLEKGITYLEFRTFDLNPFSPIGITQETVDTVHLFLLALLWIDSSSHIDQDIKEANRLNDLIALSHPLEKLPNQAPVSDLVDAMQSVIQHFNLSPYYQDLLESVKRQIQSPELTVAGQLLEMIEGLSLETFGQRQGQIYHDYAWEAPYALKGYETMELSTQLLLFDVIQKGVNFEVLDEQDQFLKLWHNSHIEYVKNGNMTSKDNYIVPLAMANKVVTKKILDEKHFPTPFGDEFTDRKEALNYFSQIQDKPIVVKPKSTNFGLGISIFKTSANLASYEKAIDIAFTEDSAILVEEYIEGTEYRFFVLEGDCIAVLLRVAANVVGDGIHTISQLVKLKNQNPLRGYDHRSPLEVIELGEVEQLMLEQQGYTVNSIPPEGTKIELRRNSNISTGGDSIDVTNTMDPTYKQLAAEMAEAMGAWVCGVDLIIPNATQAYSKDKKNATCIELNFNPLMYMHTYCQEGPGQSITPRILAKLFPEL

SEQ ID NO: 55 - DNA, 1557 bp, Escherichia coli
atgatcccggacgtatcacaggcgctggcctggctggaaaaacatcctcaggcgttaaaggggatacagcgtgggctggagcgcgaaactttgcgtgttaatgctgatggcacactggcaacaacaggtcatcctgaagcattaggttccgcactgacgcacaaatggattactaccgattttgcggaagcattgctggaattcattacaccagtggatggtgatattgaacatatgctgacctttatgcgcgatctgcatcgttatacggcgcgcaatatgggcgatgagcggatgtggccgttaagtatgccatgctacatcgcagaaggtcaggacatcgaactggcacagtacggcacttctaacaccggacgctttaaaacgctgtatcgtgaagggctgaaaaatcgctacggcgcgctgatgcaaaccatttccggcgtgcactacaatttctctttgccaatggcattctggcaagcgaagtgcggtgatatctcgggcgctgatgccaaagagaaaatttctgcgggctatttccgcgttatccgcaattactatcgtttcggttgggtcattccttatctgtttggtgcatctccggcgatttgttcttctttcctgcaaggaaaaccaacgtcgctgccgtttgagaaaaccgagtgcggtatgtattacctgccgtatgcgacctctcttcgtttgagcgatctcggctataccaataaatcgcaaagcaatcttggtattaccttcaacgatctttacgagtacgtagcgggccttaaacaggcaatcaaaacgccatcggaagagtacgcgaagattggtattgagaaagacggtaagaggctgcaaatcaacagcaacgtgttgcagattgaaaacgaactgtacgcgccgattcgtccaaaacgcgttacccgcagcggcgagtcgccttctgatgcgctgttacgtggcggcattgaatatattgaagtgcgttcgctggacatcaacccgttctcgccgattggtgtagatgaacagcaggtgcgattcctcgacctgtttatggtctggtgtgcgctggctgatgcaccggaaatgagcagtagcgaacttgcctgtacacgcgttaactggaaccgggtgatcctcgaaggtcgcaaaccgggtctgacgctgggtatcggctgcgaaaccgcacagttcccgttaccgcaggtgggtaaagatctgttccgcgatctgaaacgcgtcgcgcaaacgctggatagtattaacggcggcgaagcgtatcagaaagtgtgtgatgaactggttgcctgcttcgataatcccgatctgactttctctgcccgtatcttaaggtctatgattgatactggtattggcggaacaggcaaagcatttgcagaagcctaccgtaatctgctgcgtgaagagccgctggaaattctgcgcgaagaggattttgtagccgagcgcgaggcgtctgaacgccgtcagcaggaaatggaagccgctgataccgaaccgtttgcggtgtggctggaaaaacacgcctga

SEQ ID NO: 56 - protein, 518 aa, Escherichia coli
MIPDVSQALAWLEKHPQALKGIQRGLERETLRVNADGTLATTGHPEALGSALTHKWITTDFAEALLEFITPVDGDIEHMLTFMRDLHRYTARNMGDERMWPLSMPCYIAEGQDIELAQYGTSNTGRFKTLYREGLKNRYGALMQTISGVHYNFSLPMAFWQAKCGDISGADAKEKISAGYFRVIRNYYRFGWVIPYLFGASPAICSSFLQGKPTSLPFEKTECGMYYLPYATSLRLSDLGYTNKSQSNLGITFNDLYEYVAGLKQAIKTPSEEYAKIGIEKDGKRLQINSNVLQIENELYAPIRPKRVTRSGESPSDALLRGGIEYIEVRSLDINPFSPIGVDEQQVRFLDLFMVWCALADAPEMSSSELACTRVNWNRVILEGRKPGLTLGIGCETAQFPLPQVGKDLFRDLKRVAQTLDSINGGEAYQKVCDELVACFDNPDLTFSARILRSMIDTGIGGTGKAFAEAYRNLLREEPLEILREEDFVAEREASERRQQEMEAADTEPFAVWLEKHA

SEQ ID NO: 57 - DNA, 951 bp, Escherichia coli
atgatcaagctcggcatcgtgatggaccccatcgcaaacatcaacatcaagaaagattccagttttgctatgttgctggaagcacagcgtcgtggttacgaacttcactatatggagatgggcgatctgtatctgatcaatggtgaagcccgcgcccatacccgcacgctgaacgtgaagcagaactacgaagagtggttttcgttcgtcggtgaacaggatctgccgctggccgatctcgatgtgatcctgatgcgtaaagacccgccgtttgataccgagtttatctacgcgacctatattctggaacgtgccgaagagaaagggacgctgatcgttaacaagccgcagagcctgcgcgactgtaacgagaaactgtttaccgcctggttctctgacttaacgccagaaacgctggttacgcgcaataaagcgcagctaaaagcgttctgggagaaacacagcgacatcattcttaagccgctggacggtatgggcggcgcgtcgattttccgcgtgaaagaaggcgatccaaacctcggcgtgattgccgaaaccctgactgagcatggcactcgctactgcatggcgcaaaattacctgccagccattaaagatggcgacaaacgcgtgctggtggtggatggcgagccggtaccgtactgcctggcgcgtattccgcaggggggcgaaacccgtggcaatctggctgccggtggtcgcggtgaacctcgtccgctgacggaaagtgactggaaaatcgcccgtcagatcgggccgacgctgaaagaaaaagggctgatttttgttggtctggatatcatcggcgaccgtctgactgaaattaacgtcaccagcccaacctgtattcgtgagattgaagcagagtttccggtgtcgatcaccggaatgttaatggatgccatcgaagcacgtttacagcagcagtaa

SEQ ID NO: 58 - protein, 316 aa, Escherichia coli [sequence omitted]